(19) United States
(12) Patent Application Publication
(10) Pub. No.: US 2024/0269348 A1
(43) Pub. Date: Aug. 15, 2024

(54) Tissue-Engineered Rostral Migratory Stream for Neuronal Replacement

(71) Applicants: THE TRUSTEES OF THE UNIVERSITY OF PENNSYLVANIA, Philadelphia, PA (US); The United States of America as Represented by the Department of Veterans Affairs, Washington, DC (US)

(72) Inventors: Daniel Kacy Cullen, Media, PA (US); John C. O'Donnell, Bryn Mawr, PA (US); Erin M. Purvis, Philadelphia, PA (US); Anh Le, Philadelphia, PA (US); Qunzhou Zhang, Philadelphia, PA (US)

(21) Appl. No.: 18/566,373

(22) PCT Filed: Jun. 3, 2022
(86) PCT No.: PCT/US22/32146

§ 371 Date: Dec. 1, 2023

Related U.S. Application Data
(60) Provisional application No. 63/197,007, filed on Jun. 4, 2021.

(51) Int. Cl. A61L 27/38; A61K 35/30; A61L 27/36; C12N 5/079

(52) U.S. Cl. CPC A61L 27/3895; A61K 35/30; A61L 27/3604; A61L 27/3675; C12N 5/0622; A61L 2430/32; C12N 2501/11; C12N 2501/115; C12N 2501/135; C12N 2506/1392; C12N 2533/54; C12N 2533/76

(57) ABSTRACT

Provided herein is a method of obtaining astrocytes from gingiva-derived mesenchymal stem cells (GDMSC). Also disclosed herein are systems comprising a biocompatible construct and a plurality of astrocytes obtained from a method disclosed herein and methods of making and using the same.

### CROSS-REFERENCE TO RELATED APPLICATION

The present application is entitled to priority under 35 U.S.C. § 119(e) to U.S. Provisional Patent Application No. 63/197,007 filed Jun. 4, 2021, the content of which is incorporated by reference herein in its entirety.

### STATEMENT REGARDING FEDERALLY SPONSORED RESEARCH OR DEVELOPMENT

This invention was made with government support under NS103253 awarded by the National Institutes of Health, 1845298 awarded by the National Science Foundation, W81XWH-16-1-0796 awarded by the U.S. Army, Merit Award 101-BX003748 and IK2-RX003376 provided by the United States Government as represented by the Department of Veterans Affairs.

The government has certain rights in the invention.

### BACKGROUND

Brain injury can lead to a loss of neurons. While much work has been done to enhance neuronal recovery after brain injury, these efforts have not led to sustained improvements.

### SUMMARY

Provided herein, inter alia, is a method for obtaining an astrocyte from a gingiva-derived mesenchymal stem cell (GDMSC) and methods of using the same in a system comprising a biocompatible construct, e.g., as disclosed herein. Also provided herein are uses of a system comprising a plurality of astrocytes obtained from GDMSCs for, e.g., promoting cell migration or treating a neurodegenerative disorder or a neurological disorder, e.g., as described herein.

This disclosure provides, a method of obtaining at least one astrocyte, the method comprising: (a) providing at least one gingiva-derived mesenchymal stem cell (GDMSC), and (b) contacting at least one GDMSC with: (i) a first serum-free medium comprising N-2 supplement, basic fibroblast growth factor 2 (bFGF) and epidermal growth factor (EGF); and (ii) a second serum-free medium comprising: dibutyryl cyclic adenosine monophosphate (dbcAMP), IBMX (3-Isobutyl-1-methylxanthine), neuregulin, and platelet-derived growth factor (PDGF), wherein a GDMSC is contacted with a first medium and/or a second medium under conditions sufficient to induce differentiation of the GDMSC into an astrocyte.

In some embodiments, a GDMSC is obtained from a subject, e.g., a human.

In some embodiments, a GDMSC is obtained from a population of cells in which at least 75% cells are cranial neural crest derived mesenchymal stem cells (e.g., at least 75%, 80%, 85%, 90%, 95%, 96%, 97%, 98% or 99%). In some embodiments, a GDMSC is obtained from a population of cells in which no more than 25% cells are mesoderm derived mesenchymal stem cells (e.g. no more than 20%, 15%, 10% 5%, or 1%). In some embodiments, a GDMSC is obtained from a population of cells in which at least 75% cells are cranial neural crest derived mesenchymal stem cells and no more than 25% cells are mesoderm derived mesenchymal stem cells.

In some embodiments, a neural crest derived mesenchymal stem cell has an increased ability to differentiate into neuronal cells as compared to mesoderm derived mesenchymal stem cells. In some embodiments, a neural crest derived mesenchymal stem cell has an increased immunomodulatory capacity compared to mesoderm derived mesenchymal stem cells.

In some embodiments, a GDMSC is contacted with a first medium for about 72 hours.

In some embodiments, a GDMSC is contacted with a second medium for about 72 hours.

In some embodiments, a first medium and/or a second medium further comprises one or more additional components. In some embodiments, one or more additional components comprises: an antibiotic, an amino acid supplement, an anti-fungal, or a combination thereof.

In some embodiments, a first medium comprises about 20 ng/ml of N-2 supplement, about 20 ng/ml basic fibroblast growth factor 2 (bFGF) and about 20 ng/ml epidermal growth factor (EGF).

In some embodiments, a second medium comprises about 0.5 to 1 mM dibutyryl cyclic adenosine monophosphate (dbcAMP), about 0.5 to 1 mM IBMX (3-Isobutyl-1-methylxanthine), about 50 ng/ml to 100 ng/ml of neuregulin, and about 1 ng/ml to 5 ng/ml of platelet-derived growth factor (PDGF).

In some embodiments, a GDMSC disclosed herein comprises one or more of the following characteristics:
(i) express Oct4, SSEA-4, Stro-1, CD29, CD73, CD90, CD105, Type I collagen, or a combination thereof;
(ii) has low or no detectable expression of CD45;
(iii) is capable of forming colonies in a colony formation assay;
(iv) is capable of differentiating into one or more progenitor cells such as adipocytes and osteoblasts;
(v) is capable of suppressing proliferation of T cells;
(vi) is capable of suppressing proliferation of T cells by expressing one or more soluble mediators such as IDO and IL-10;
(vii) is capable of proliferating in response to a stimuli, e.g., a drug or pathogen; and/or
(viii) is capable of inducing an immune response, e.g., a pro-inflammatory immune response.

In some embodiments, a GDMSC disclosed herein comprises any one of (i) to (viii).

In some embodiments, a GDMSC disclosed herein comprises any two of (i) to (viii).

In some embodiments, a GDMSC disclosed herein comprises any three of (i) to (viii).

In some embodiments, a GDMSC disclosed herein comprises any four of (i) to (viii).

In some embodiments, a GDMSC disclosed herein comprises any five of (i) to (viii).

In some embodiments, a GDMSC disclosed herein comprises any six of (i) to (viii).

In some embodiments, a GDMSC disclosed herein comprises any seven of (i) to (viii).

In some embodiments, a GDMSC disclosed herein comprises all of (i) to (viii).

In some embodiments, of any of the methods disclosed herein, the amount of time required to obtain an astrocyte is reduced relative to a comparator. In some embodiments, a comparator comprises an otherwise similar method using induced pluripotent stem cells (iPSC) to obtain an astrocyte.

In some embodiments, the amount of time required to obtain an astrocyte is less than 1 week, less than 6 days, less than 5 days, or less than 4 days.

In some embodiments, of any of the methods disclosed herein, an astrocyte obtained from a method disclosed herein comprises one or more of the following characteristics:
(i) expresses GFAP, Ezrin, Robo2, S-100-beta, glutamine synthetase (GS) and/or GLutamate ASpartate Transporter (GLAST);
(ii) expresses pyruvate carboxylase (PC) and/or Glutamate transporter-1 (GLT-1);
(iii) has low or no expression of an endothelial marker, e.g., CD31;
(iv) is capable of self-assembly into a bundle of longitudinally-aligned astrocytes with bipolar or multipolar processes,
(v) is capable of forming a bundle of astrocytes; and/or
(vi) has a morphology of an astrocyte.

In some embodiments, an astrocyte obtained from a method disclosed herein comprises any one of (i)-(vi).

In some embodiments, an astrocyte obtained from a method disclosed herein comprises any two of (i)-(vi).

In some embodiments, an astrocyte obtained from a method disclosed herein comprises any three of (i)-(vi).

In some embodiments, an astrocyte obtained from a method disclosed herein comprises any four of (i)-(vi).

In some embodiments, an astrocyte obtained from a method disclosed herein comprises any five of (i)-(vi).

In some embodiments, an astrocyte obtained from a method disclosed herein comprises all of (i)-(vi).

In some embodiments, a bundle of astrocytes comprises a structure similar or substantially similar to a structure of an astrocyte in an endogenous rostral migratory stream.

In some embodiments, a bundle of astrocytes comprises a function similar to or substantially similar to a function of an astrocyte in an endogenous rostral migratory stream.

Also provided herein is a reaction mixture comprising at least one GDMSC and a serum-free medium comprising N-2 supplement, basic fibroblast growth factor 2 (bFGF) and epidermal growth factor (EGF).

In some embodiments, a reaction mixture further comprises one or more additional components. In some embodiments, one or more additional components comprises: an antibiotic, an amino acid supplement, an anti-fungal, or a combination thereof.

This disclosure further provides, a reaction mixture comprising at least one GDMSC and a medium comprising: dibutyryl cyclic adenosine monophosphate (dbcAMP), IBMX (3-Isobutyl-1-methylxanthine), neuregulin, and platelet-derived growth factor (PDGF).

In some embodiments, a reaction mixture further comprises one or more additional components. In some embodiments, one or more additional components comprises: an antibiotic, an amino acid supplement, an anti-fungal, or a combination thereof.

In some embodiments, of a reaction mixtures disclosed herein, a reaction mixture is maintained under conditions sufficient to obtain an astrocyte.

In some embodiments, of a reaction mixtures disclosed herein, an astrocyte obtained from a reaction mixture comprises one or more of the following characteristics:
(i) expresses GFAP, Ezrin, Robo2, S-100-beta, glutamine synthetase (GS) and/or GLutamate ASpartate Transporter (GLAST);
(ii) expresses pyruvate carboxylase (PC) and/or Glutamate transporter-1 (GLT-1);
(iii) has low or no expression of an endothelial marker, e.g., CD31;
(iv) is capable of self-assembly into a bundle of longitudinally-aligned astrocytes with bipolar or multipolar processes,
(v) is capable of forming a bundle of astrocytes; and/or
(vi) has a morphology of an astrocyte.

In some embodiments, of a reaction mixtures disclosed herein, a bundle of astrocytes comprises a structure similar or substantially similar to a structure of an astrocyte in an endogenous rostral migratory stream.

In some embodiments, of a reaction mixtures disclosed herein, a bundle of astrocytes comprises a function similar to or substantially similar to a function of an astrocyte in an endogenous rostral migratory stream.

Further provided herein is a system, comprising: (a) a plurality of astrocytes derived from at least one gingiva-derived mesenchymal stem cell (GDMSC); and (b) a biocompatible construct comprising a matrix and having a first end, a second end and a body.

In some embodiments, a plurality of astrocytes used in a system disclosed herein is obtained according to a method disclosed herein.

In some embodiments, a system disclosed herein further comprises a Slit-Robo entity. In some embodiments, a Slit-Robo entity is or comprises an agent that promotes activation and/or signaling from a Slit-Robo pathway.

In some embodiments, a system disclosed herein is characterized in that when implanted into an organism, it promotes migration of one or more cells compared to a comparator. In some embodiments, a comparator comprises an otherwise similar system without a plurality of astrocytes; or without a plurality of astrocytes derived from at least one GDMSC.

In some embodiments, one or more migrating cells comprises one or more endogenous host cells. In some embodiments, an endogenous host cell comprises a neural precursor cell, a neuroblast, a neuron, a progenitor cell, a glial cell, an astrocyte, and/or an endothelial cell.

In some embodiments, one or more migrating cells comprises: one or more cells provided in the system; or one or more cells derived from, differentiated from or a progenitor of a cell provided in the system.

In some embodiments, migration of one or more cells occurs within the system, throughout the system, out of the system or into the system.

In some embodiments, migration of one or more cells occurs to the site of implantation, or away from the site of implantation.

In some embodiments of a system disclosed herein, a matrix of a biocompatible construct comprises an inner surface and an outer surface.

In some embodiments, an inner surface of the biocompatible construct defines a luminal core.

In some embodiments, an outer surface of the biocompatible construct comprises at least one hydrogel.

In some embodiments, a hydrogel comprises one or more of agarose, hyaluronic acid, chitosan, alginate, collagen, dextran, pectin, carrageenan, polylysine, gelatin, hyaluronic acid, fibrin, and methylcellulose.

In some embodiments, a hydrogel comprises hyaluronic acid. In some embodiments, a hyaluronic acid is or comprises methacrylated HA (MeHA).

In some embodiments, a hydrogel is or comprises agarose. In some embodiments, an agarose is at about 0.25-30%, about 0.25%-3%, about 0.5%-3%, about 1-20%, about 1.5-10%, about 2-9%, about 2.5-8%, or about 3-7%. In some embodiments, an agarose is at about 3%.

In some embodiments, an inner surface of the biocompatible construct comprises one or more extracellular matrix (ECM) components.

In some embodiments, an ECM component comprises collagen, laminin, fibronectin, hyaluronic acid, or a combination thereof.

In some embodiments, an ECM comprises collagen. In some embodiments, a collagen is at a concentration of about 0.1-10 mg/ml or 0.1-9 mg/ml. In some embodiments, a collagen is at a concentration of about 1 mg/ml.

In some embodiments, a plurality of astrocytes is seeded at least once in a system disclosed herein.

In some embodiments, a plurality of astrocytes is seeded twice in a system disclosed herein.

In some embodiments, a plurality of astrocytes is seeded at a cell density of about 0.1 million cells/ml to about 10 million cells/ml. In some embodiments, a plurality of astrocytes is seeded at a cell density of about 1 million cells/ml.

In some embodiments, a plurality of astrocytes comprises at least 500 cells, at least 1000 cells, at least 5000 cells, at least 10,000 cells, at least 15,000 cells, at least 20,000 cells, at least 40,000 cells, at least 80,000 cells, at least 100,000 cells, or at least 500,000 cells.

In some embodiments, a system disclosed herein further comprises one or more additional cells or components. In some embodiments, an additional cell is not an astrocyte, e.g., as described herein. In some embodiments, an additional cell is or comprises an endothelial cell. In some embodiments, a one or more additional cells or components induces vascularization.

In some embodiments, an additional cell or component is introduced into the system: (i) concurrently with, before or after seeding of the plurality of astrocytes; (ii) during formation of an astrocyte bundle by the plurality of astrocytes; and/or (ii) after formation of an astrocyte bundle by the plurality of astrocytes.

Disclosed herein is a method of manufacturing a system comprising: (a) a biocompatible construct comprising a matrix and having a first end, a second end and a body; and (b) associating the biocompatible construct with a plurality of astrocytes derived from at least one gingiva-derived mesenchymal stem cell (GDMSC).

In some embodiments, a method of manufacturing comprises maintaining a system under conditions that promotes growth of at least one astrocyte in a plurality of astrocytes.

In some embodiments, a method of manufacturing comprises maintaining a system under conditions that maintain viability of at least one astrocyte in a plurality of astrocytes.

In some embodiments, a method of manufacturing comprises forming an aggregate of at least a portion of the plurality of astrocytes.

In some embodiments, a system in a method of manufacturing disclosed herein comprises any one of a system disclosed herein.

Provided herein is a method of promoting cell migration, comprising:
providing a system comprising: (a) a plurality of astrocytes derived from at least one gingiva-derived mesenchymal stem cell; and (b) a biocompatible construct comprising a first end, a second end and a body comprising a luminal core,
wherein a method is characterized in that when implanted in an organism, it promotes migration of one or more cells through or along the luminal core of a biocompatible construct.

In some embodiments, a method of promoting cell migration comprises implanting a system disclosed herein in a subject. In some embodiments, a system is implanted: (i) at, near or within a brain lesion in a subject; (ii) at, near or within an area in the brain with insufficient neurons or neuronal connections, or with damaged neurons or neuronal connections; (iii) at, near or within a subventricular zone, an endogenous rostral migratory stream or a neurogenic niche; and/or (iv) at, near or within a region of a brain affected by a brain injury, a neurodegenerative disease or disorder, or a neurodevelopmental disease or disorder.

In some embodiments, one or more migrating cells comprises one or more endogenous host cells.

In some embodiments, an endogenous host cell comprises a neural precursor cell, a neuroblast, a neuron, a progenitor cell, a glial cell, an astrocyte, and/or an endothelial cell.

In some embodiments, one or more migrating cells comprises: one or more cells provided in a system, or one or more cells derived from, differentiated from or a progenitor of a cell provided in a system.

In some embodiments, migration of one or more cells occurs within a system, throughout a system, out of a system or into a system.

In some embodiments, migration of one or more cells occurs a site of implantation, or away from a site of implantation.

This disclosure also provides a method of treating a neurodegenerative disorder or a neurological disorder in a subject, comprising providing to a subject a system comprising: (a) a plurality of astrocytes derived from at least one gingiva-derived mesenchymal stem cell (GDMSC); and (b) a biocompatible construct comprising a matrix and having a first end, a second end and a body.

Also provided herein is a method of ameliorating one or more symptoms of a neurodegenerative disorder or a neurological disorder in a subject, comprising providing to a subject a system comprising: (a) a plurality of astrocytes derived from at least one gingiva-derived mesenchymal stem cell (GDMSC); and (b) a biocompatible construct comprising a matrix and having a first end, a second end and a body.

In some embodiments, a neurodegenerative disorder comprises a disorder with injury or degeneration to one or more neurons.

In some embodiments, a neurodegenerative disorder comprises brain injury or spinal cord injury.

In some embodiments, brain injury comprises acute brain injury, degenerative brain injury, traumatic brain injury (TBI), or chronic brain injury.

In some embodiments, a neurological disorder comprises: Parkinson's, Alzheimer's, Huntington's, prion disease, motor neuron disease, spinocerebellar ataxia, spinal muscular atrophy, amyotrophic lateral sclerosis (ALS), encephalitis, epilepsy, head and brain malformations, or hydrocephalus.

In some embodiments, a subject is a human.

In some embodiments of a method disclosed herein, a system disclosed herein is implanted into a subject. In some embodiments, a system is implanted into a brain of a subject.

In some embodiments, a system is implanted: (i) at, near or within a brain lesion in a subject; (ii) at, near or within an area in a brain with insufficient neurons or neuronal connections, or with damaged neurons or neuronal connections; (iii) at, near or within a subventricular zone, an endogenous rostral migratory stream or a neurogenic niche; and/or (iv) at, near or within a region of a brain affected by a brain injury, a neurodegenerative disease or disorder, or a neurodevelopmental disease or disorder.

In some embodiments of a method disclosed herein, a system is characterized in that when implanted into an organism it ameliorates or reduces severity of one or more symptoms of a disorder.

In some embodiments of a method disclosed herein, a system is characterized in that when implanted into an organism it promotes migration of one or more cells.

In some embodiments, migration of one or more cells is compared to a comparator. In some embodiments, a comparator comprises an organism implanted with an otherwise similar system without a plurality of astrocytes; or without a plurality of astrocytes derived from at least one GDMSC.

In some embodiments, one or more migrating cells comprises one or more endogenous host cells.

In some embodiments, an endogenous host cell comprises a neural precursor cell, a neuroblast, a neuron, a progenitor cell, a glial cell, an astrocyte, and/or an endothelial cell.

In some embodiments, one or more migrating cells comprises: one or more cells provided in a system; or one or more cells derived from, differentiated from or a progenitor of a cell provided in a system.

In some embodiments, migration of one or more cells occurs within a system, throughout a system, out of a system or into a system.

In some embodiments, migration of one or more cells occurs to a site of implantation, or away from a site of implantation.

Also provided herein is a kit comprising, a system comprising: (a) a plurality of astrocytes derived from at least one gingiva-derived mesenchymal stem cell (GDMSC); (b) a biocompatible construct comprising a matrix and having a first end, a second end and a body; and (c) instructions for using the same.

In some embodiments, a plurality of astrocytes is obtained according to a method disclosed herein.

### BRIEF DESCRIPTION OF THE DRAWINGS

FIGS. 1A-1H show a physiological inspiration and exemplary therapeutic application of the tissue-engineered rostral migratory stream (TE-RMS). Sagittal view of a rodent brain (FIG. 1A) depicting the endogenous rostral migratory stream (FIG. 1B). Neural precursor cells continue to be produced in in the subventricular zone of most adult mammals. These cells can mature to into neuroblasts and migrate in chains along the pathway of aligned astrocytes that comprise the rostral migratory stream to arrive at the olfactory bulb. In the presence of a lesion, neuroblasts divert from the endogenous SVZ/RMS and migrate toward the lesion, but their numbers are not sufficient to improve functional recovery (FIG. 1C). The TE-RMS is comprised of tight bundles of longitudinally aligned astrocytes within a hydrogel microcolumn. Immature neurons seeded on one end of the TE-RMS migrate as chains through the TERMS in vitro (FIG. 1D). Migrating neurons release Slit1, which is recognized by the Robo2 receptors that are expressed by the astrocytes comprising the TE-RMS (FIG. 1E). This chemorepellent communication allows the neuroblasts to efficiently migrate through the aligned astrocyte network and serves as one example of the dynamic bidirectional communication that occurs in the endogenous RMS. The TE-RMS can be extracted from its hydrogel microcolumn and implanted into the rodent brain to span the distance between the SVZ/RMS and the lesion (FIG. 1F). Proof-of-principle evidence suggests that neuroblasts will divert from the SVZ/RMS and migrate in chain formation through the implanted TE-RMS (FIG. 1G). Based on existing literature, it is predicted that over time redirected neuroblasts will mature into phenotype-relevant mature neurons in lesioned regions and integrate into existing circuitry (FIG. 1H).

FIGS. 2A-2R show characteristic protein enrichment in RMS astrocytes relative to surrounding protoplasmic astrocytes in the rat brain. Formalin-fixed, paraffin-embedded (FFPE) rat brains were sagittally-sectioned and immunostained for GFAP (FIGS. 2A-2F), Ezrin (FIGS. 2G-2L), and Robo2 (FIGS. 2M-2R). GFAP, Ezrin, and Robo2 channels from a representative image are displayed in a wide view containing a portion of RMS and surrounding brain (FIGS. 2A, 2G, and 2M), with standardized regions of interest (ROIs) annotated within for both RMS and protoplasmic (Proto) astrocytes. Enlarged ROIs are provided for each channel (FIGS. 2B-2C, 2H-2I, 2N-2O). Automated binary masks were generated from GFAP ROIs (FIGS. 2D-2E) to allow for isolation of astrocytic signal from the ROIs of each channel. Images resulting from application of these astrocytic GFAP masks to ROIs are provided for the Ezrin (FIGS. 2J-2K) and Robo2 (FIG. 2P-2Q) channels. Mean intensities were quantified from ROIs after astrocytic signal isolation, and intensity values for the Proto/RMS ROI pairs from each image were compared by paired Student's t-test for GFAP (FIG. 2F), Ezrin (FIG. 2L), and Robo2 (FIG. 2R). Intensity values normalized to the Proto measurements for each pair are displayed for all 5 animals. *p<0.05. Scale bars: 200 microns (FIGS. 2A, 2G, 2M), 50 microns (FIGS. 2B, 2C, 2H, 2I, 2N, 2O).

FIGS. 3A-3P demonstrate that TE-RMSs fabricated from rat astrocytes are enriched in GFAP, Ezrin, and Robo2 relative to planar sister cultures. Primary rat astrocytes were passaged, split, and either plated in a planar collagen matrix or used for TE-RMS fabrication. Cell nuclei were labelled with Hoechst stain, and cells were immunostained for GFAP, Ezrin, and Robo2. Representative wide views of merged fluorescent channels are provided for planar sister cultures (FIG. 3A) and TE-RMS (FIG. 3B), with call-out boxes providing magnified views of planar (FIG. 3C) and TE-RMS (FIG. 3D) organization, morphology, and relative protein. Maximum contrast white-on-black single-channel images along with quantification of normalized intensities are provided for Hoechst (FIGS. 3E-3G), Ezrin (FIGS. 3H-3J), GFAP (FIGS. 3K-3M), and Robo2 (FIGS. 3N-3P). Values are displayed as mean±SEM. Means were compared by Student's t-test. **p<0.01, ***p<0.005. Scale bars: 250 microns.

FIGS. 4A-4N show astrocytes derived from adult human gingiva mesenchymal stem cells (GMSC) can be used for TE-RMS fabrication. A representative image of human GMSC-derived astrocytes in planar culture is provided with merged fluorescent channels (FIG. 4A), and maximum contrast white-on-black single-channel images are provided for Hoechst staining of nuclei (FIG. 4B) and immunostaining that demonstrates expression of astrocytic proteins Glutamine Synthetase (GS) (FIG. 4C), Glutamate/Aspartate Transporter (GLAST) (FIG. 4D), and GFAP (FIG. 4E). A merged fluorescent image is also provided at higher magnification with alternative staining targets (FIG. 4F), with maximum contrast white-on-black single-channel images for Hoechst staining of nuclei (FIG. 4G) and immunostaining that demonstrates expression of astrocytic proteins S100B (FIG. 4H) and GFAP (FIG. 4I), but not endothelial marker CD31 (FIG. 4J). Western blot analysis from three donors before and after astrocyte induction, demonstrating increased expression of astrocytic proteins GFAP and GS, with GAPDH loading control (FIG. 4K). A representative TE-RMS fabricated using the human GMSC-derived astrocytes as a starting biomass was labelled with Hoechst nuclear stain, immunostained for GFAP, Ezrin, and Robo2, and imaged via laser confocal microscopy. (FIGS. 4L-4N) Single z plane overlay illustrating the bidirectional morphology and longitudinal alignment of astrocytes comprising the human TE-RMS. Maximum contrast white-on-black single z plane images of individual channels at high magnification demonstrate the presence and plasma membrane localization of Ezrin (FIG. 4M), and Robo2 (FIG. 4N) proteins known to be enriched in glial tube astrocytes. Scale bars: 200 microns (FIGS. 4A-4J), 50 microns (FIGS. 4L-4N).

FIGS. 5A-5P demonstrate TE-RMSs fabricated from adult human gingiva derived astrocytes are enriched in GFAP, Ezrin, and Robo2 relative to planar sister cultures. Astrocytes derived from adult human gingiva stem cells were passaged, split, and either plated in a planar collagen matrix or used for TE-RMS fabrication. Cell nuclei were labelled with Hoechst stain, and cells were immunostained for GFAP, Ezrin, and Robo2. Representative wide views of merged fluorescent channels are provided for planar sister cultures (FIG. 5A) and TE-RMS (FIG. 5B), with call-out boxes providing magnified views of planar (FIG. 5C) and TE-RMS (FIG. 5D) organization, morphology, and relative protein. Maximum contrast white-on-black single-channel images along with quantification of normalized intensities are provided for Hoechst (FIGS. 5E-5G), Ezrin (FIGS. 5H-5J), GFAP (FIGS. 5K-5M), and Robo2 (FIGS. 5N-5P). Values are displayed as mean±SEM. Means were compared by Student's t-test. *p<0.05, **p<0.01. Scale bars: 250 microns.

FIGS. 6A-6H show that migration of immature rat neurons is facilitated by human TE-RMSs in vitro. Immature neuronal aggregates prepared from rat cortex were inserted in one end of human GMSC-derived TE-RMSs and acellular collagen controls, and these assembled in vitro migration assays were then fixed 72 hours later for immunolabeling and analyses. Compressed z stacks of stitched confocal images are displayed in a wide view with all channels merged, consisting of Hoechst (nuclei) and Tuj1 (neurites) channels, along with either Collagen in the representative acellular collagen control (FIG. 6A), Laminin in the representative acellular collagen/laminin control (FIG. 6B), or Human nuclei and GFAP in the representative human TE-RMS (FIG. 6C). Call-out boxes provide magnified views proximal to the aggregate in each column (a′-c′). Quantification of area of Hoechst-positive, Human-negative nuclei (nuclei from the immature neuronal aggregate) is provided for all groups for 0-1 mm from the aggregate (FIG. 6D) and 1-3.5 mm from the aggregate (FIG. 6E). Data are displayed as mean±SEM with points to indicate individual sample values; n=4, 5, and 4 for Coll, Lam+Coll, and TE-RMS, respectively (**p<0.005, ***p<0.001 with Bonferroni correction for multiple comparisons). A single z plane of a stitched confocal image from a representative human TE-RMS containing Hoechst, Human Nuclei, Tuj1, and GFAP channels is displayed in a wide view with all channels merged (FIG. 6F), with just the nuclear labels (FIG. 6G), and with just the astrocyte and neuron specific cytoskeleton labels (FIG. 6H). Call-out boxes provide magnified views along the TE-RMS proximal to the aggregate (f′-h′), ˜2.5 mm from the aggregate (f†-h†), and ˜3.5 mm from the aggregate (f‡-h‡). Opaque outlines in g′ highlight the narrow path for chain migration forged by immature neurons through the TE-RMS. White arrows in g† and g‡ indicate the Hoechst+/Human− nuclei of immature neurons migrating the length of the TE-RMS. Scale bars: 500 microns (FIGS. 6A-6C; 6F-6H), 250 microns (a′-c′; f-h‡).

FIGS. 7A-7I shows that implantation of human TE-RMSs in the brains of athymic rats demonstrates surgical feasibility and proof of principle for redirecting neuroblast migration. Pairs of human GMSC-derived TE-RMSs and acellular collagen controls were bilaterally implanted into the brains of athymic rats using precise stereotaxic coordinates to span RMS and cortex. Images captured during (FIG. 7A) and after (FIG. 7B) bi-lateral stereotactic implantation of TE-RMS. Gross pathology of formalin fixed brain from top (FIG. 7C) and side (FIG. 7D) view (note that D is blocked to show the implant trajectory). Immunolabelling rat GFAP demonstrating accurate placement of TE-RMS contacting the RMS. (FIG. 7E) Immunolabelling showing colabeling of collagen within the acellular control implant and DCX positive host cells present in the surrounding tissue but absent from the collagen implant midway through the column (FIG. 7F; ˜3 mm from RMS), and present in the collagen at the interface with the endogenous RMS (FIG. 7G). Immunolabelling showing non-overlapping colabeling of human nuclei of the TE-RMS astrocytes and DCX positive (human negative) host neuroblasts migrating through the TE-RMS and midway through the implant (FIG. 7H; ˜ 3 mm from RMS; white arrows indicate DCX+/Human− cells) and at the interface between TE-RMS and host RMS (FIG. 7I). Scale bars: 500 microns.

FIGS. 8A-8L Tissue-engineered rostral migratory stream astrocytes possess unique cellular morphology compared to planar astrocytes. Phase microscope image of planar astrocytes in culture (FIG. 8A) with magnified view depicting planar astrocyte morphology in higher detail (FIG. 8B). Planar astrocytes possess round nuclei and have multidirectional processes that extend radially around the cell (FIG. 8C). TE-RMSs are fabricated in hollow agarose microcolumns (FIG. 8D) that are loaded with collagen (FIG. 8E) and then following collagen polymerization are seeded with a concentrated astrocyte suspension (FIGS. 8F, 8G). Astrocytes pull the polymerized collagen off the inner lumen of the microcolumn and use it to align themselves in longitudinal bundles tethered to either end of the microcolumn (FIGS. 8H, 8I). TE-RMS astrocytes possess elongated nuclei and bidirectional processes that preferentially extend in parallel with the microcolumn (FIG. 8J). Magnified phase images depict these longitudinally aligned bundles of astrocytes exhibiting this unique morphology (FIGS. 8K, 8L). Scale bars: 200 microns (FIGS. 8A, 8G, 8K), 50 microns (FIG. 8B), 500 microns (FIG. 8I), 100 microns (FIG. 8L).

FIGS. 9A-9K Quantification of nuclear and cytoskeletal measurements. Representative fluorescent image of single planar astrocyte (FIG. 9A) depicting nuclear (Hoechst, blue) and cytoskeleton (GFAP, green) labels. To perform nuclear length measurements, Hoechst channel was isolated (FIG. 9B) and FIJI “find edges” function was applied (FIG. 9C). To measure the long nuclear axis, a line was drawn across the longest distance from one edge of the nucleus to another (FIG. 9D). To measure the short nuclear axis, a line was drawn from one edge of the nucleus to the other and perpendicular to the long nuclear axis (FIG. 9E). GFAP channel was isolated (FIG. 9F). Main processes were GFAP extensions arising directly from the nucleus (FIG. 9G) and branch points were GFAP extensions arising from other GFAP extensions (FIG. 9H). To perform angle measurements, long and short nuclear axes were viewed in image depicting Hoechst and GFAP channels (FIG. 9I). An angle was drawn with the first point of the angle placed on the cell process (at the point where the process contacted the cell body), the second point (middle) of the angle was placed on the intersection of the cell's long and short nuclear axes, and the third point of the angle placed on the end of the long nuclear axis (the end closer to the cell process) (FIG. 9J, 9K). Scale bar: 50 microns (FIG. 9A). *: main processes (FIG. 9G) and branch points (FIG. 9H).

FIGS. 10A-10J TE-RMS astrocytes exhibit distinct cytoskeletal architecture compared to planar astrocytes. Representative 20× fluorescent image of planar astrocyte culture (FIG. 10A) with zoom-in on one single astrocyte depicting nuclear (Hoechst, blue) and cytoskeleton (GFAP, green) labels (FIG. 10B) and just the cytoskeleton label (FIG. 10C). Representative 20× fluorescent image of TE-RMS (FIG. 10D) with zoom-in on one single astrocyte depicting nuclear (Hoechst) and cytoskeleton (GFAP) labels (FIG. 10E) and just the cytoskeleton label (FIG. 10F). Nested 1-test analysis of number of main cell processes in independent planar (n=6) versus TE-RMS (n=9) cultures (FIG. 10G). Each point represents a single cell and bars represent the median of each group. Nested 1-test analysis of number of branch points in independent planar and TE-RMS cultures (FIG. 10H). Each point represents a single cell and bars represent the median of each group. The angle of each main process was measured as it deviates from the long nuclear axis. Nested 1-test analysis with each point representing the median angle from each cell and the graph bars representing the median of each group (FIG. 10I). All quantified angles (n=1069 planar; n=644 TE-RMS) (FIG. 10J). ****p<0.0001. Scale bars: 200 microns (FIGS. 10A, 10D), 50 microns (FIGS. 10B, 10C. 10E, 10F).

FIGS. 11A-11I Endogenous RMS astrocytes exhibit distinct cytoskeletal architecture compared to surrounding protoplasmic astrocytes. Schematic depicting sagittal rat brain slice (FIG. 11A) and the subventricular zone-rostral migratory stream-olfactory bulb pathway (FIG. 11B). Representative fluorescent image of a sagittal rat brain slice with nuclear Hoechst stain (blue) and immunostaining for GFAP (green) at 20× magnification (FIG. 11C). Call out boxes highlight the cytoskeleton of a protoplasmic astrocyte (FIG. 11D) and RMS astrocytes (FIG. 11E). Nested 1-test analysis of the number of main cell process possessed by protoplasmic versus RMS astrocytes (n=5, within-subjects) (FIG. 11F). Each point represents a single cell and bars represent the median of each group. Nested 1-test analysis of the number of branch points possessed by protoplasmic and RMS astrocytes (FIG. 11G). Each point represents a single cell and bars represent the median of each group. The angle of each main process was measured as it deviates from the long nuclear axis. Nested 1-test analysis with each point representing the median angle from each cell and the graph bars representing the median of each group (FIG. 11H). Each point represents the median of each cell and bars represent the median of each group. All quantified angles (n=585 protoplasmic; n=303 RMS) (FIG. 11I) (i). ****p<0.0001, **p<0.01. Scale bars: 200 microns (FIG. 11C), 40 microns (FIGS. 11D, 11E).

FIGS. 12A-12H TE-RMS astrocytes have elongated nuclei compared to planar astrocytes. Representative 20× fluorescent image of planar astrocyte culture (FIG. 12A) with zoom-in on one single astrocyte depicting nuclear (Hoechst, blue) and cytoskeleton (GFAP, green) labels (FIG. 12B) and just nuclear label (FIG. 12C). Representative 20× fluorescent image of TE-RMS (FIG. 12D) with zoom-in on one single astrocyte depicting nuclear (Hoechst) and cytoskeleton (GFAP) labels (FIG. 12E) and just nuclear label (FIG. 12F). Example planar nucleus (FIG. 12C) and non-overlapping TE-RMS nuclei (FIG. 12F) are outlined in white. Nested 1-test analysis of nuclear aspect ratio of planar astrocytes versus TE-RMS astrocytes (FIG. 12G). Each point represents a single cell and bars represent the median of each group. Frequency distribution of the nuclear aspect ratio of planar and TE-RMS astrocytes (FIG. 12H). ****p<0.0001. Scale bars: 200 microns (FIG. 12A, 12D), 50 microns (FIGS. 12B, 12C, 12E, 12F).

FIGS. 13A-13E Endogenous RMS astrocytes have elongated nuclei compared to surrounding protoplasmic astrocytes. Representative fluorescent image of a sagittal rat brain slice with nuclear Hoechst stain (blue) and immunostaining for GFAP (green) at 20× magnification (FIG. 13A) highlighting the nuclei of a protoplasmic astrocyte (FIG. 13B) and several RMS astrocytes (FIG. 13C). Example nuclei are outlined in white (FIG. 13B, 13C). Nested t-test analysis of nuclear aspect ratios of protoplasmic astrocytes versus RMS astrocytes (FIG. 13D). Each point represents a single cell and bars represent the median of each group. Frequency distribution of the nuclear aspect ratio of endogenous protoplasmic and RMS astrocytes (FIG. 13E). ***p=0.0007. Scale bars: 200 microns (FIG. 13A), 40 microns (FIG. 13B, 13C).

FIGS. 14A-14H High magnification fluorescent imaging depicting novel astrocyte morphology. High magnification (100×) fluorescent imaging highlighting differences in nuclear shape and intermediate filament arrangement between single planar astrocytes (FIGS. 14A-14F) and TE-RMS astrocytes (FIG. 14G, 14H). Nuclei (Hoechst) depicted in blue and intermediate filaments (GFAP) in green. All images are compressed confocal z-stacks. Scale bars: 50 microns (FIG. 14A-14H).

FIGS. 15A-15I Scanning electron microscopy imaging depicting novel astrocyte morphology. SEM imaging of planar astrocytes (FIGS. 15A-15C) and TE-RMSs (FIG. 15D-15I). Single planar astrocytes (FIGS. 15A-15C) display process complexity and heterogeneity in morphology. Full length TE-RMS (FIG. 15D) with magnified view (FIG. 15E) depicting longitudinally aligned, bundled astrocyte processes coated in a fine meshwork of collagen. Single elongated cell with bidirectional processes visible within TE-RMS bundle (FIG. 15F). TE-RMS with four distinct astrocytes with visibly connected processes (FIG. 15G). Magnified views (FIG. 15H, 15I) depicting two cells (FIG. 15H) and one cell (FIG. 15I) visible on top of collagen network that encompasses TE-RMS construct. Black arrows (FIGS. 15F-15I) indicate individual cell bodies visible within the TE-RMS, highlighting their distinct morphology. Scale bars: 10 microns (FIG. 15I), 30 microns (FIG. 15H), 50 microns (FIG. 15A, 15B, 15E, 15F), 100 microns (FIG. 15C, 15G), 1 mm (FIG. 15D).

### DEFINITIONS

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, “synapse” refers to a junction between a neuron and another cell, across which chemical communication flows.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

In general, the term “engineered” refers to the aspect of having been manipulated by the hand of man. For example, a cell or organism is considered to be “engineered” if it has been subjected to a manipulation, so that its genetic, epigenetic, and/or phenotypic identity is altered relative to an appropriate reference cell such as otherwise identical cell that has not been so manipulated In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered cell are typically still referred to as “engineered” even though the actual manipulation was performed on a prior entity.

As used herein, the term “subject” refers to an organism, typically a mammal (e.g., a human). In some embodiments, a subject is suffering from a disease, disorder, and/or condition. In some embodiments, a subject is susceptible to a disease, disorder, and/or condition. In some embodiments, a subject displays one or more symptoms or characteristics of a disease, disorder, and/or condition. In some embodiments, a subject is someone with one or more features characteristic of susceptibility to or risk of a disease, disorder, and/or condition. In some embodiments, a subject is a patient. In some embodiments, a subject is an individual to whom diagnosis and/or therapy is and/or has been administered.

The term “biocompatible construct”, as used herein, refers to a construct comprising a matrix that does not cause significant harm to living tissue when placed in contact with such tissue, e.g., in vivo (e.g., over a period of time). In certain embodiments, a biocompatible construct is not toxic to cells (e.g., over a period of time). In certain embodiments, a biocompatible construct does not induce significant inflammation or other immune response, or other adverse effects when placed or implanted into a subject (e.g., over a period of time). In some embodiments, a matrix of a biocompatible construct comprises a scaffold. In some embodiments, a matrix of a biocompatible construct comprises an inner surface, an outer surface or both an inner surface and an outer surface. In some embodiments, an inner surface of a matrix of a biocompatible construct defines a luminal core. In some embodiments, an inner surface of a matrix of a biocompatible construct comprises a component disclosed herein. In some embodiments, an outer surface of a matrix of a biocompatible construct comprises a component disclosed herein. In some embodiments, a biocompatible construct comprises one or more additional components.

The term “astrocyte,” as used herein refers to a non-neuronal cell which supports proliferation, differentiation, signaling and/or homeostasis of a neuronal cell. In some embodiments, an astrocyte is present in a central nervous system of an organism. In some embodiments, an astrocyte is obtained from a central nervous system of an organism. In some embodiments, an astrocyte is derived from a stem cell, e.g., a mesenchymal stem cell, e.g., a GDMSC. In some embodiments, an astrocyte has one or more of the following characteristics: (i) expresses GFAP, Ezrin, Robo2, S-100-beta, glutamine synthetase (GS) and/or Glutamate Aspartate Transporter (GLAST); (ii) expresses pyruvate carboxylase (PC) and/or Glutamate transporter-1 (GLT-1); (iii) has low or no expression of an endothelial marker, e.g., CD31; (iv) is capable of self-assembly into a bundle of longitudinally-aligned astrocytes with bipolar or multipolar processes, (v) is capable of forming a bundle of astrocytes; and/or (vi) has a morphology of an astrocyte. In some embodiments, an astrocyte is capable of self-assembly into a bundle of longitudinally aligned astrocytes with bipolar (e.g., bidirectional) processes. In some embodiments, an astrocyte has a “star-like” morphology, e.g., as observed in protoplasmic astrocytes. In some embodiments, an astrocyte has a linear morphology, e.g., as observed in fibrous astrocytes. In some embodiments, an astrocyte has any one of characteristics (i) to (vi). In some embodiments, an astrocyte has any two of characteristics (i) to (vi). In some embodiments, an astrocyte has any three of characteristics (i) to (vi). In some embodiments, an astrocyte has any four of characteristics (i) to (vi). In some embodiments, an astrocyte has any five of characteristics (i) to (vi). In some embodiments, an astrocyte has all of characteristics (i) to (vi).

### DETAILED DESCRIPTION OF CERTAIN EMBODIMENTS

The present disclosure identifies certain challenges that can be associated with regeneration of neurons upon injury to the brain. For example, the present disclosure identifies certain problems that can be encountered with inducing neuronal regeneration and/or migration of nascent neurons to a site of injury.

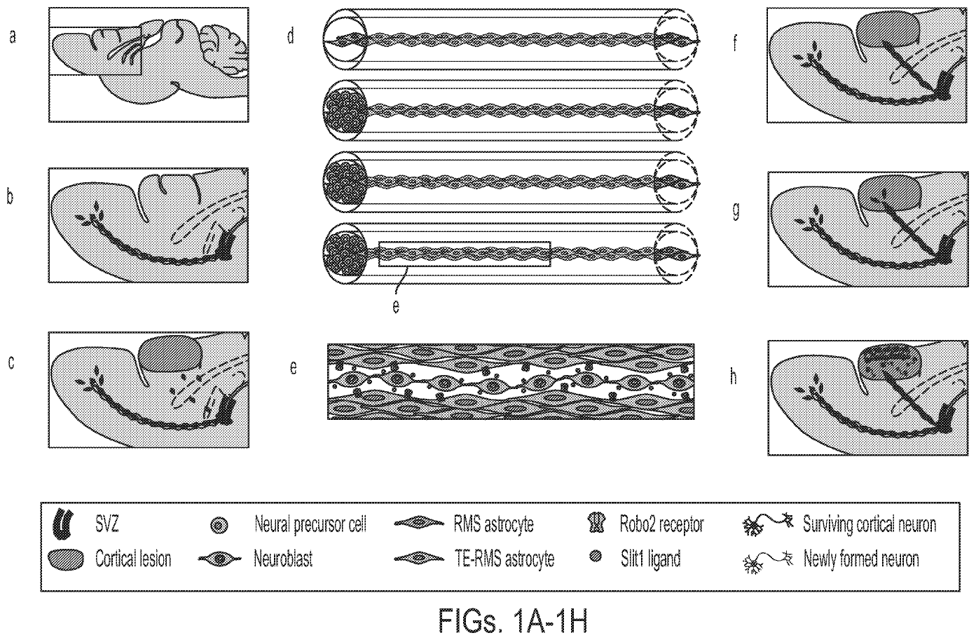

Brain injury can cause loss of neurons and endogenous neurogenesis is upregulated in the subventricular zone (SVZ) following brain injury (16-18). In adults, during neurogenesis neural precursor cells (NPCs) in the SVZ can differentiate into neuroblasts and migrate through the rostral migratory stream (RMS) to the olfactory bulb (OB) where they mature into interneurons and integrate into existing circuitry (3-6). Increased neurogenesis has been reported in the rodent SVZ following multiple experimental models of acquired brain injury. Following brain injury, newly formed NPCs can mature into neuroblasts, divert from the SVZ/RMS, and migrate toward injured brain regions (27, 28, 34, 35)(FIG. 1C). However, the quantity of SVZ-derived cells that mature into functional neurons in injured regions appears insufficient to improve functional recovery at physiological levels (26, 36).

Neural tissue engineering has introduced the possibility of developing customized therapies to enhance neuronal regeneration following traumatic brain injury. A variety of biomaterial and tissue engineering technologies have been developed to enhance the neurogenic potential of the SVZ and redirect the migration of SVZ neuroblasts to neuron-deficient brain regions following various experimental brain injuries. The evidence from tissue engineering techniques, along with that of pharmacological and genetic approaches, collectively demonstrates that experimental intervention to enhance the brain's intrinsic repair mechanism to replace lost or damaged neurons with endogenous SVZ NPCs can improve recovery after acquired brain injury. However, while promising, these interventions have thus far only afforded transient re-direction of neuroblasts.

Among other things, the present disclosure provides technologies for improving functional recovery upon brain injury. For example, this disclosure provides novel methods for generating astrocytes from gingiva-derived mesenchymal stem cells (GDMSC), a source of mesenchymal stem cells that is easily accessible, and more homogenous, e.g., as compared to oral mucosal stem cell (OMSC). In some embodiments, a GDMSC is obtained from a population of cells having at least 75% (e.g., at least 80%, 85%, 90%, 95%, 99%) neural crest derived stem cells. In some embodiments, an astrocyte obtained from gingiva-derived mesenchymal stem cells (GDMSC), can be used in a system disclosed herein (e.g., a TE-RMS) to promote neuronal regeneration, proliferation and/or migration.

In some embodiments, a system disclosed herein, e.g., a TE-RMS, promotes migration of a cell (e.g., an endogenous cell, e.g., an NPC or a neuroblast) through a TE-RMS to a site of injury. In some embodiments, a system disclosed herein, e.g., a TE-RMS, promotes or supports differentiation of a precursor cell, e.g., an NPC, into a neuroblast. Without wishing to be bound by theory, the present disclosure proposes that a system disclosed herein, e.g., a TE-RMS, redirects migration of endogenous neuroblasts out of the RMS and into distal lesions, to provide sustained delivery to replace lost neurons and improve functional recovery. In some embodiments, a system disclosed herein, e.g., a TE-RMS disclosed herein, emulates a brain's known method for transporting neuroblasts to a distal area for neuronal replacement. In some embodiments, a sustained influx of new neurons is likely required for functional improvements across a spectrum of brain injury severities.

### Method of Obtaining Astrocytes

Among other things, this disclosure provides a method of obtaining an astrocyte from a gingiva-derived mesenchymal stem cell (GDMSC). In some embodiments, a method of obtaining an astrocyte from a GDMSC comprises (a) providing at least one gingiva-derived mesenchymal stem cell (GDMSC), and (b) contacting at least one GDMSC with certain serum-free media, under conditions sufficient to induce differentiation of a GDMSC into an astrocyte.

In some embodiments, a method of obtaining an astrocyte disclosed herein comprises contacting a GDMSC with a first serum-free medium comprising N-2 supplement, basic fibroblast growth factor 2 (bFGF) and epidermal growth factor (EGF). In some embodiments, a method disclosed herein comprises contacting a GDMSC with a further, e.g., a second, particular serum-free medium.

In some embodiments, a method of obtaining an astrocyte disclosed herein comprises contacting a GDMSC with a second serum-free medium comprising: dibutyryl cyclic adenosine monophosphate (dbcAMP), IBMX (3-Isobutyl-1-methylxanthine), neuregulin, and platelet-derived growth factor (PDGF). In some embodiments, a method disclosed herein comprises contacting a GDMSC with a different, e.g., a first, serum-free medium.

In some embodiments, a first serum-free medium comprises about 5-40 ng/ml, about 5-35 ng/ml, about 5-30 ng/ml, about 5-25 ng/ml, about, 5-20, ng/ml, about 10-40 ng/ml, about 15-30 ng/ml, about 15-25 ng/ml, about 15-20 ng/ml of N-2 supplement. In some embodiments, a first serum-free medium comprises about 20 ng/ml of N-2 supplement.

In some embodiments, a first serum-free medium comprises about 5-40 ng/ml, about 5-35 ng/ml, about 5-30 ng/ml, about 5-25 ng/ml, about, 5-20, ng/ml, about 10-40 ng/ml, about 15-30 ng/ml, about 15-25 ng/ml, about 15-20 ng/ml of basic fibroblast growth factor 2 (bFGF). In some embodiments, a first serum-free medium comprises about 20 ng/ml of basic fibroblast growth factor 2 (bFGF).

In some embodiments, a first serum-free medium comprises about 5-40 ng/ml, about 5-35 ng/ml, about 5-30 ng/ml, about 5-25 ng/ml, about, 5-20, ng/ml, about 10-40 ng/ml, about 15-30 ng/ml, about 15-25 ng/ml, about 15-20 ng/ml of epidermal growth factor (EGF). In some embodiments, a first serum-free medium comprises about 20 ng/ml of epidermal growth factor (EGF).

In some embodiments, a first serum-free medium comprises about 20 ng/ml of N-2 supplement, about 20 ng/ml basic fibroblast growth factor 2 (bFGF) and about 20 ng/ml epidermal growth factor (EGF).

In some embodiments, a method of obtaining an astrocyte disclosed herein comprises contacting a GDMSC with a first serum-free medium comprising N-2 supplement, basic fibroblast growth factor 2 (bFGF) and epidermal growth factor (EGF) and a second serum-free medium comprising: dibutyryl cyclic adenosine monophosphate (dbcAMP), IBMX (3-Isobutyl-1-methylxanthine), neuregulin, and platelet-derived growth factor (PDGF).

In some embodiments, a second medium comprises about 0.5 to 1 mM, about 0.6 to 1 mM, about 0.7 to 1 mM, about 0.8 to 1 mM, about 0.9 to 1 mM dibutyryl cyclic adenosine monophosphate (dbcAMP). In some embodiments, a second medium comprises about 0.5 mM, about 0.6 mM, about 0.7 mM, about 0.8 mM, about 0.9 mM, or about 1 mM dibutyryl cyclic adenosine monophosphate (dbcAMP).

In some embodiments, a second medium comprises about 0.5 to 1 mM, about 0.6 to 1 mM, about 0.7 to 1 mM, about 0.8 to 1 mM, about 0.9 to 1 mM IBMX (3-Isobutyl-1-methylxanthine). In some embodiments, a second medium comprises about 0.5 mM, about 0.6 mM, about 0.7 mM, about 0.8 mM, about 0.9 mM, or about 1 mM IBMX (3-Isobutyl-1-methylxanthine).

In some embodiments, a second medium comprises about 50 ng/ml to 100 ng/ml, about 60 ng/ml to 100 ng/ml, about 70 ng/ml to 100 ng/ml, about 80 ng/ml to 100 ng/ml, about 90 ng/ml to 100 ng/ml, about 95 ng/ml to 100 ng/ml of neuregulin. In some embodiments, a second medium comprises about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml of neuregulin.

In some embodiments, a second medium comprises about 1 ng/ml to 5 ng/ml, about 1.5 to 5 ng/ml, about 2 to 5 ng/ml, about 2.5 to 5 ng/ml, about 3 to 5 ng/ml, about 3.5 to 5 ng/ml, about 4 to 5 ng/ml, about 4.5 to 5 ng/ml of platelet-derived growth factor (PDGF). In some embodiments, a second medium comprises about 1 ng/ml, about 1.5 ng/ml, about 2 ng/ml, about 2.5 ng/ml, about 3 ng/ml, about 3.5 ng/ml, about 4 ng/ml, about 4.5 ng/ml, about 5 ng/ml of platelet-derived growth factor (PDGF).

In some embodiments, a second medium comprises about 0.5 to 1 mM dibutyryl cyclic adenosine monophosphate (dbcAMP), about 0.5 to 1 mM IBMX (3-Isobutyl-1-methylxanthine), about 50 ng/ml to 100 ng/ml of neuregulin, and about 1 ng/ml to 5 ng/ml of platelet-derived growth factor (PDGF).

In some embodiments, a first medium and/or a second medium further comprises one or more additional components. In some embodiments, one or more additional components comprises: an antibiotic, an amino acid supplement, an anti-fungal, or a combination thereof.

In some embodiments, a GDMSC is contacted with a first medium for about 65 hours, about 66 hours, about 67 hours, about 68 hours, about 69 hours, about 70 hours, about 71 hours, about 72 hours, about 73 hours, about 74 hours, about 75 hours, about 76 hours, about 77 hours, about 78 hours, about 79 hours or about 80 hours. In some embodiments, a GDMSC is contacted with a first medium for about 65-80 hours, for about 65-75 hours, for about 68-75 hours, or for about 70-75 hours. In some embodiments, a GDMSC is contacted with a first medium for about 72 hours.

In some embodiments, a GDMSC is contacted with a second medium for about 65 hours, about 66 hours, about 67 hours, about 68 hours, about 69 hours, about 70 hours, about 71 hours, about 72 hours, about 73 hours, about 74 hours, about 75 hours, about 76 hours, about 77 hours, about 78 hours, about 79 hours or about 80 hours. In some embodiments, a GDMSC is contacted with a second medium for about 65-80 hours, for about 65-75 hours, for about 68-75 hours, or for about 70-75 hours. In some embodiments, a GDMSC is contacted with a second medium for about 72 hours.

In some embodiments of a method of obtaining an astrocyte described herein, the amount of time required to obtain an astrocyte is reduced relative to a comparator. In some embodiments, a comparator comprises an otherwise similar method using induced pluripotent stem cells (iPSC) to obtain an astrocyte.

In some embodiments of a method of obtaining an astrocyte described herein, the amount of time required to obtain an astrocyte the amount of time required to obtain an astrocyte is less than 1 week, less than 6 days, less than 5 days, or less than 4 days. In some embodiments, the amount of time required to obtain an astrocyte is less than 1 week. In some embodiments, the amount of time required to obtain an astrocyte is less than 6 days. In some embodiments, the amount of time required to obtain an astrocyte is less than 5 days, In some embodiments, the amount of time required to obtain an astrocyte is less than 4 days.

In some embodiments, the amount of time required to obtain an astrocyte is about 1 week. In some embodiments, the amount of time required to obtain an astrocyte is about 6 days. In some embodiments, the amount of time required to obtain an astrocyte is about 5 days. In some embodiments, the amount of time required to obtain an astrocyte is about 4 days.

In some embodiments, a method of obtaining an astrocyte described herein comprises culturing a GDMSC at about 37 degrees Celsius. In some embodiments, a method of obtaining an astrocyte described herein comprises culturing a GDMSC in about 5% CO2.

In some embodiments, a method of obtaining an astrocyte described herein further comprises culturing an astrocyte obtained from a method disclosed herein under conditions sufficient to maintain viability and/or induce proliferation of an astrocyte. In some embodiments, an astrocyte obtained from a method disclosed herein is cultured in media, e.g., DMEM/F12 medium, supplemented with serum, e.g., 10% FBS, and an antibiotic, e.g., 1% Penicillin-Streptomycin.

In some embodiments, an astrocyte obtained from a method disclosed herein comprises one or more characteristics of an astrocyte, e.g., as described herein. In some embodiments, an astrocyte has one or more of the following characteristics: (i) expresses GFAP, Ezrin, Robo2, S-100-beta, glutamine synthetase (GS) and/or GLutamate ASpartate Transporter (GLAST); (ii) expresses pyruvate carboxylase (PC) and/or Glutamate transporter-1 (GLT-1); (iii) has low or no expression of an endothelial marker, e.g., CD31; (iv) is capable of self-assembly into a bundle of longitudinally-aligned astrocytes with bipolar or multipolar processes, (v) is capable of forming a bundle of astrocytes; and/or (vi) has a morphology of an astrocyte. In some embodiments, an astrocyte is capable of self-assembly into a bundle of longitudinally aligned astrocytes with bidirectional (e.g., bipolar) or multidirectional processes. In some embodiments, an astrocyte has a “star-like” morphology, e.g., as observed in protoplasmic astrocytes. In some embodiments, an astrocyte has a linear morphology, e.g., as observed in fibrous astrocytes.

In some embodiments, an astrocyte obtained from a method disclosed herein is cultured for about 1-20 passages, about 2-15 passages, or about 4-10 passages.

In some embodiments, a culture of an astrocyte obtained from a method disclosed herein is subjected to perturbation, e.g., mechanical perturbation. In some embodiments, mechanical perturbation of a culture of an astrocyte obtained from a method disclosed herein allows for purification of an astrocyte population. In some embodiments, a culture of an astrocyte is mechanically perturbed prior to trypisinization.

### Gingiva-Derived Mesenchymal Stem Cells

Mesenchymal stem cells derived from human gingiva were first described by Zhang Q Z et al., (2009) Journal of Immunology 183:7787-7798, the entire contents of which are hereby incorporated by reference. Such gingiva-derived mesenchymal stem cells (GDMSC) comprise stem-cell like properties and immunomodulatory properties. Additional properties of GDMSCs such as GDMSCs comprising a population of cells that is primarily derived from cranial neural crest stem cells were previously disclosed by Xu X. et al., (2013) J. Dent Res 92(9): 825-832, the entire contents of which are hereby incorporated by reference.

In part, the present disclosure encompasses the surprising finding that GDMSCs can differentiate into astrocytes. GDMSCs possess several characteristics that are not shared by other mesenchymal stem cells (MSCs). For example, GDMSCs exhibit a proliferative capacity beyond that of other MSCs, including bone marrow-derived MSCs and umbilical cord-derived MSCs, and are also capable of potent immunomodulatory effects. Further, GDMSCs exhibit the potential to differentiate into a variety of cell types including neurons, keratinocytes, and endothelial cells. See Kim et al., (2021) Gingiva-Derived Mesenchymal Stem Cells: Potential Application in Tissue Engineering and Regenerative Medicine—A Comprehensive Review, Frontiers in Immunology, 12: 667221. As is shown in the examples below, the present disclosure describes, inter alia, the creation of astrocytes and their use in new tissue engineered constructs.

Furthermore, Zhang Q Z et al., (2012) disclosed that gingiva, from which GDMSCs are derived, is a unique microenvironment fueled by food residues, microbial flora and saliva, and is recognized for, e.g., its sensitivity to inflammation, fibrosis response and proneness to drug-induced overgrowth (See Zhang Q Z et al., (2012) J Dent Res 91(11): 1011-1018).

Accordingly, in some embodiments, a GDMSC disclosed herein is obtained from a population of cells that is relatively homogenous, e.g., as compared to oral mucosal stem cells (OMSC).

In some embodiments, a GDMSC is obtained from a population of cells in which at least 75% cells, at least 76% cells, at least 77% cells, at least 78% cells, at least 79% cells, at least 80% cells, at least 81% cells, at least 82% cells, at least 83% cells, at least 85% cells, at least 86% cells, at least 87% cells, at least 88% cells, at least 89% cells, at least 90% cells, at least 91%, at least 92%, at least 93%, at least 94%, at least 95%, at least 96% at least 97%, at least 98%, at least 99% cells are cranial neural crest derived mesenchymal stem cells. In some embodiments, about 75% to 100%, or about 80% to 100%, about 85% to 100%, about 90% to 100%, or about 91% to 100%, about 92% to 100%, about 93% to 100%, about 94% to 100%, about 95% to 100%, about 96% to 100%, about 97% to 100%, about 98% to 100%, about 99% to 100%, about 75% to 99%, about 75% to 98%, about 75% to 97%, about 75% to 96% about 75% to 95%, about 75% to 94%, about 75% to 93%, about 75% to 92%, or about 75% to 91% cells, about 75% to 90%, about 75% to 85%, or about 75% to 80% cells are cranial neural crest derived mesenchymal stem cells.

In some embodiments, a GDMSC is obtained from a population of cells in which no more than 25%, no more than 20%, no more than 15%, no more than 10%, no more than 9%, no more than 8%, no more than 7%, no more than 6%, no more than 5%, no more than 4%, no more than 3%, no more than 2% or no more than 1% cells are mesoderm derived mesenchymal stem cells.

In some embodiments, a GDMSC is obtained from a population of cells in which at least 75% cells are cranial neural crest derived mesenchymal stem cells and no more than 25% cells are mesoderm derived mesenchymal stem cells.

In some embodiments, a GDMSC is obtained from a population of cells in which at least 80% cells are cranial neural crest derived mesenchymal stem cells and no more than 20% cells are mesoderm derived mesenchymal stem cells.

In some embodiments, a GDMSC is obtained from a population of cells in which at least 85% cells are cranial neural crest derived mesenchymal stem cells and no more than 15% cells are mesoderm derived mesenchymal stem cells.

In some embodiments, a GDMSC is obtained from a population of cells in which at least 90% cells are cranial neural crest derived mesenchymal stem cells and no more than 10% cells are mesoderm derived mesenchymal stem cells.

In some embodiments, a GDMSC is obtained from a population of cells in which at least 95% cells are cranial neural crest derived mesenchymal stem cells and no more than 5% cells are mesoderm derived mesenchymal stem cells.

In some embodiments, a GDMSC is obtained from a population of cells in which at least 99% cells are cranial neural crest derived mesenchymal stem cells and no more than 1% cells are mesoderm derived mesenchymal stem cells.

In some embodiments, a neural crest derived mesenchymal stem cell has an increased ability to differentiate into neuronal cells as compared to mesoderm derived mesenchymal stem cells.

In some embodiments, a neural crest derived mesenchymal stem cell has an increased immunomodulatory capacity compared to mesoderm derived mesenchymal stem cells. In some embodiments, an immunomodulatory capacity comprises: (i) increase in number and/or activity of T regulatory cells; (ii) decrease in number and/or activity of inflammatory T cells such as Th17 cells; (iii) increase in number and/or activity of cytotoxic T cells, or a combination thereof.

In some embodiments, a GDMSC comprises one or more of the following characteristics:
(i) express Oct4, SSEA-4, Stro-1, CD29, CD73, CD90, CD105, Type I collagen, or a combination thereof;
(ii) has low or no detectable expression of CD45;
(iii) is capable of forming colonies in a colony formation assay;
(iv) is capable of differentiating into one or more progenitor cells such as adipocytes and osteoblasts;
(v) is capable of suppressing proliferation of T cells;
(vi) is capable of suppressing proliferation of T cells by expressing one or more soluble mediators such as IDO and IL-10;
(vii) is capable of proliferating in response to a stimuli, e.g., a drug or pathogen; (viii) is capable of inducing an immune response, e.g., a pro-inflammatory immune response; and/or
(ix) is capable of reducing the severity and/or one or more symptoms of DSS-induced colitis, e.g., when administered systemically in an animal model.

In some embodiments, a GDMSC comprises any one of (i)-(ix).

In some embodiments, a GDMSC comprises any two of (i)-(ix).

In some embodiments, a GDMSC comprises any three of (i)-(ix).

In some embodiments, a GDMSC comprises any four of (i)-(ix).

In some embodiments, a GDMSC comprises any five of (i)-(ix).

In some embodiments, a GDMSC comprises any six of (i)-(ix).

In some embodiments, a GDMSC comprises any seven of (i)-(ix).

In some embodiments, a GDMSC comprises any eight of (i)-(ix).

In some embodiments, a GDMSC comprises all of (i)-(ix).

### Astrocytes

Astrocytes are cells with distinctive morphological and functional characteristics that depend on the location of the cells in the central nervous system or brain. In general, astrocytes have a regulatory role and have been shown to play a role, e.g., in neurogenesis and synaptogenesis; controlling blood-brain barrier permeability; and maintaining extracellular homeostasis (see Siracusa R. et al. (2019) Front. Pharmacol. 10:1114, the entire contents of which are hereby incorporated by reference). One of the functions of astrocytes is in synaptic regulation, e.g., in the formation and maturation of synapses; and/or in the maintenance, pruning and remodeling of synapses during development, aging and/or diseases (see Matias I., et al (2019) Front. Aging Neurosci. 11:59, the entire contents of which are hereby incorporated by reference). Due in part to the heterogeneity of astrocytes, these cells are also implicated in a wide array of neuropathologies.

Exemplary astrocytes, e.g., as disclosed herein, include protoplasmic astrocytes, fibrous astrocytes, varicose astrocytes, interlaminar projection astrocytes, radial glial cells, Bergmann glia and Muller glia. In some embodiments, an astrocyte has one or more of the following characteristics: (i) expresses GFAP, Ezrin, Robo2, S-100-beta, glutamine synthetase (GS) and/or GLutamate ASpartate Transporter (GLAST); (ii) expresses pyruvate carboxylase (PC) and/or Glutamate transporter-1 (GLT-1); (iii) has low or no expression of an endothelial marker, e.g., CD31; (iv) is capable of self-assembly into a bundle of longitudinally-aligned astrocytes with bipolar or multipolar processes, (v) is capable of forming a bundle of astrocytes; and/or (vi) has a morphology of an astrocyte. In some embodiments, an astrocyte is capable of self-assembly into a bundle of longitudinally aligned astrocytes with bidirectional (e.g., bipolar) or multidirectional processes. In some embodiments, an astrocyte has a “star-like” morphology, e.g., as observed in protoplasmic astrocytes. In some embodiments, an astrocyte has a linear morphology, e.g., as observed in fibrous astrocytes.

In some embodiments, an astrocyte has any one of characteristics (i) to (vi). In some embodiments, an astrocyte has any two of characteristics (i) to (vi). In some embodiments, an astrocyte has any three of characteristics (i) to (vi). In some embodiments, an astrocyte has any four of characteristics (i) to (vi). In some embodiments, an astrocyte has any five of characteristics (i) to (vi). In some embodiments, an astrocyte has all of characteristics (i) to (vi).

In some embodiments, an astrocyte is a cell that expresses GFAP, Ezrin, Robo2, S-100-beta, glutamine synthetase (GS) and/or Glutamate Aspartate Transporter (GLAST), or a combination thereof. In some embodiments, an astrocyte is a cell that expresses GFAP. In some embodiments, an astrocyte is a cell that expresses Ezrin. In some embodiments, an astrocyte is a cell that expresses Robo2. In some embodiments, an astrocyte is a cell that expresses S-100-beta. In some embodiments, an astrocyte is a cell that expresses glutamine synthetase (GS). In some embodiments, an astrocyte is a cell that expresses Glutamate Aspartate Transporter (GLAST). In some embodiments, an astrocyte is a cell that expresses GFAP, Ezrin, Robo2, S-100-beta, glutamine synthetase (GS) and Glutamate Aspartate Transporter (GLAST),

In some embodiments, an astrocyte is obtained, e.g., derived, from a stem cell, e.g., a GDMSC.

In some embodiments, an astrocyte is obtained from a central nervous system of an organism.

In some embodiments, an astrocyte is obtained using a method disclosed herein.

### Systems Comprising a Biocompatible Construct and GMSC Derived Astrocytes

Disclosed herein, inter alia, are systems comprising: a biocompatible construct comprising a matrix; and a plurality of astrocytes, e.g., obtained from a GDMSC. Systems disclosed herein can be manufactured, e.g., assembled, in vitro or ex vivo. Systems disclosed herein can be implanted in a subject and can be used to induce regeneration, proliferation, differentiation and/or migration of a cell, e.g., a progenitor cell, a neuronal precursor cell, a neuroblast, a neuron, a glial cell, an astrocyte, and/or an endothelial cell. Systems disclosed herein can also be used to treat and/or ameliorate one or more symptoms of a neurodegenerative disorder or a neurological disorder in a subject. In some embodiments, a system disclosed herein is referred to as a Tissue Engineered Rostral Migratory Stream (TE-RMS).

In some embodiments, a system disclosed herein (e.g., a TE-RMS) comprises a plurality of astrocytes obtained according to a method disclosed herein. In some embodiments, a plurality of astrocytes in a system disclosed herein has one or more characteristics of an astrocyte, e.g., as disclosed herein.

In some embodiments, at least a portion of a plurality of astrocytes in a system disclosed herein aggregate, e.g., form a bundle, e.g., a bundle of astrocytes. In some embodiments, a substantial portion of a plurality of astrocytes in a system disclosed herein aggregate, e.g., form a bundle, e.g., a bundle of astrocytes. In some embodiments, a plurality of astrocytes in a system disclosed herein aggregate, e.g., form a bundle, e.g., a bundle of astrocytes.

In some embodiments, a plurality of astrocytes in a system disclosed herein comprise a bidirectional (e.g., bipolar) or multidirectional morphology.

In some embodiments, a system disclosed herein (e.g., a TE-RMS) further comprises one or more additional cells and/or one or more additional components. In some embodiments, an additional cell or component is introduced into a system: (i) concurrently with, before or after seeding of the plurality of astrocytes; (ii) during formation of an astrocyte bundle by the plurality of astrocytes; and/or (ii) after formation of an astrocyte bundle by the plurality of astrocytes.

In some embodiments, one or more additional components comprises a Robo-Slit entity. In some embodiments, a Slit-Robo entity is or comprises an agent that promotes activation and/or signaling from a Slit-Robo pathway

In some embodiments, one or more additional cells or components induces vascularization.

In some embodiments, a system disclosed herein further comprises one or more additional cells. In some embodiments, an additional cell is a cell other than an astrocyte or astrocytic cell. In some embodiment, an additional cell in a system disclosed herein induces vascularization. In some embodiments, an additional cell is an endothelial cell.

Also disclosed herein is a method of manufacturing a system disclosed herein, e.g., a TE-RMS. In some embodiments, a method of manufacturing a system disclosed herein, e.g., a TE-RMS, comprises (a) providing a biocompatible construct comprising a matrix and having a first end, a second end and a body; and (b) associating a biocompatible construct with a plurality of astrocytes derived from at least one gingiva-derived mesenchymal stem cell (GDMSC).

In some embodiments, associating a biocompatible construct with a plurality of astrocytes comprises seeding a plurality of astrocytes in a biocompatible construct. In some embodiments, a plurality of astrocytes is seeded on an inner surface of a biocompatible construct.

In some embodiments, a plurality of astrocyte is seeded at least once in a biocompatible construct. In some embodiments, a plurality of astrocyte is seeded twice in a biocompatible construct.

In some embodiments, a plurality of astrocytes is seeded at a cell density of about 0.1 million cells/ml to about 10 million cells/ml. In some embodiments, a plurality of astrocytes is seeded at a cell density of about 0.1 million cells/ml, about 0.2 million cells/ml, about 0.5 million cells/ml, about 1 million cells/ml, about 2 million cells/ml, about 3 million cells/ml, about 4 million cells/ml, about 5 million cells/ml, about 6 million cells/ml, about 7 million cells/ml, about 8 million cells/ml, about 9 million cells/ml, or about 10 million cells/ml. In some embodiments, a plurality of astrocytes is seeded at a cell density of about 1 million cells/ml.

In some embodiments, a plurality of astrocytes, e.g., in a system disclosed herein, comprises at least 500 cells, at least 1000 cells, at least 5000 cells, at least 10,000 cells, at least 15,000 cells, at least 20,000 cells, at least 40,000 cells, at least 80,000 cells, at least 100,000 cells, or at least 500,000 cells.

In some embodiments, a method of manufacturing a system disclosed herein comprises maintaining a system under conditions that promotes growth of at least one astrocyte in the plurality of astrocytes. In some embodiments, a method of manufacturing a system disclosed herein comprises maintaining a system under conditions that maintain viability of at least one astrocyte in the plurality of astrocytes.

In some embodiments, a method of manufacturing a system disclosed herein comprises forming an aggregate, e.g., a bundle of astrocytes, of at least a portion of the plurality of astrocytes.

Additional methods of manufacturing a system disclosed herein, e.g., a TE-RMS, are disclosed in International Application PCT/US2015/065353 filed on Dec. 11, 2015, the entire contents of which are hereby incorporated by reference.

In some embodiments, disclosed herein is a system comprising a plurality of astrocytes wherein at least a portion of said astrocytes comprises one or more aligned and/or elongated astrocyte processes. In some embodiments, an astrocyte process has a length of at least 0.2 mm, at least 0.5 mm, at least 1 mm, at least 2 mm, at least 5 mm or at least 10 mm. Also disclosed herein is a method of manufacturing a system comprising a plurality of astrocytes wherein at least a portion of said astrocytes comprise one or more aligned and/or elongated astrocyte processes.

In some embodiments, a system disclosed herein is a three-dimensional system.

### Biocompatible Constructs

A biocompatible construct disclosed herein comprises a matrix, e.g., as disclosed herein, and a first end, a second end, and a body. In some embodiments, a matrix of a biocompatible construct comprises an inner surface and/or an outer surface. In some embodiments, an inner surface of a biocompatible construct defines a luminal core.

In some embodiments, a matrix of a biocompatible construct comprises a scaffold. In some embodiments, a matrix of a biocompatible construct comprises a component described herein, e.g., a component of an inner surface as described herein, and/or a component of an outer surface as described herein. In some embodiments, a matrix of a biocompatible construct comprises one or more additional components, e.g., as disclosed herein.

As an example, an outer surface of a biocompatible construct disclosed herein comprises at least one hydrogel (e.g., as described herein). In some embodiments, a hydrogel comprises a hydrophilic biopolymer and/or a synthetic polymer. In some embodiments, a hydrogel is at least partially cross-linked, wherein the cross-linking optionally increases stiffness, reduces porosity, and/or increases degradation time. In some embodiments, a hydrophilic biopolymer comprises one or more of agarose, hyaluronic acid, chitosan, alginate, collagen, dextran, pectin, carrageenan, polylysine, gelatin, hyaluronic acid, fibrin, and methylcellulose.

In some embodiments, a hydrophilic biopolymer comprises agarose. In some embodiments, a hydrophilic biopolymer comprises hyaluronic acid. In some embodiments, a hydrophilic biopolymer comprises chitosan. In some embodiments, a hydrophilic biopolymer comprises alginate. In some embodiments, a hydrophilic biopolymer comprises collagen. In some embodiments, a hydrophilic biopolymer comprises dextran. In some embodiments, a hydrophilic biopolymer comprises pectin. In some embodiments, a hydrophilic biopolymer comprises carrageenan. In some embodiments, a hydrophilic biopolymer comprises polylysine. In some embodiments, a hydrophilic biopolymer comprises gelatin. In some embodiments, a hydrophilic biopolymer comprises hyaluronic acid. In some embodiments, a hydrophilic biopolymer comprises fibrin. In some embodiments, a hydrophilic biopolymer comprises methylcellulose.

In some embodiments, a hydrophilic biopolymer comprises hyaluronic acid. In some embodiments, wherein a hyaluronic acid is or comprises methacrylated HA (MeHA).

In some embodiments, a hydrophilic biopolymer comprises agarose. In some embodiments, an agarose is at about 0.25-30%, about 0.25%-3%, about 0.5%-3%, about 1-20%, about 1.5-10%, about 2-9%, about 2.5-8%, or about 3-7%. In some embodiments, the agarose is at about 0.25-29%, about 0.25-28%, 0.25-%, about 0.25-27%, about 0.25-26%, about 0.25-25%, about 0.25-24%, about 0.25-23%, about 0.25-22%, about 0.25-21%, about 0.25-20%, about 0.25-19%, about 0.25-18%, about 0.25-17%, about 0.25-16%, about 0.25-15%, about 0.25-14%, about 0.25-13%, about 0.25-12%, about 0.25-11%, about 0.25-10%, about 0.25-9%, about 0.25-8%, about 0.25-7%, about 0.25-6%, about 0.25-5%, about 0.25-4%, about 0.25-3%, about 0.25-2%, about 0.25-1%, about 0.25-0.5%, about 0.5-30%, about 1-30%, about 2-30%, about 3-30%, about 4-30%, about 5-30%, about 6-30%, about 7-30%, about 8-30%, about 9-30%, about 10-30%, about 11-30%, about 12-30%, about 13-30%, about 14-30%, about 15-30%, about 16-30%, about 17-30%, about 18-30%, about 19-30%, about 20-30%, about 21-30%, about 22-30%, about 23-30%, about 24-30%, about 25-30%, about 26-30%, about 27-30%, about 28-30%, about 29-30%.

In some embodiments, an agarose is at about 0.25%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%. In some embodiments, an agarose is at about 3%.

An inner surface of a biocompatible construct disclosed herein, in some embodiments, comprises one or more extracellular matrix (ECM) components. In some embodiments, an ECM component comprises collagen, laminin, fibronectin, hyaluronic acid, or a combination thereof. In some embodiments, an ECM component comprises collagen. In some embodiments, an ECM component comprises laminin. In some embodiments, an ECM component comprises fibronectin. In some embodiments, an ECM component comprises hyaluronic acid.

In some embodiments, an ECM component comprises collagen. In some embodiments, a collagen is at a concentration of about 0.1-10 mg/ml. In some embodiments, a collagen is at a concentration of about 0.1-9 mg/ml, about 0.1-8 mg/ml, about 0.1-7 mg/ml, about 0.1-6 mg/ml, about 0.1-5 mg/ml, about 0.1-4 mg/ml, about 0.1-3 mg/ml, about 0.1-2 mg/ml, about 0.1-1 mg/ml, about 0.1-0.9 mg/ml, about 0.1-0.8 mg/ml, about 0.1-0.7 mg/ml, about 0.1-0.5 mg/ml, about 0.5-10 mg/ml, about 0.6-10 mg/ml, about 0.7-10 mg/ml, about 0.8-10 mg/ml, about 0.9-10 mg/ml, about 1-10 mg/ml, about 2-10 mg/ml, about 3-10 mg/ml, about 4-10 mg/ml, about 5-10 mg/ml, about 6-10 mg/ml, about 7-10 mg/ml, about 8-10 mg/ml, about 9-10 mg/ml. In some embodiments, a collagen is at a concentration of about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml. In some embodiments, a collagen is at a concentration of about 1 mg/ml.

In some embodiments, an ECM component comprises laminin. In some embodiments, a laminin is at a concentration of about 0.1-9 mg/ml, about 0.1-8 mg/ml, about 0.1-7 mg/ml, about 0.1-6 mg/ml, about 0.1-5 mg/ml, about 0.1-4 mg/ml, about 0.1-3 mg/ml, about 0.1-2 mg/ml, about 0.1-1 mg/ml, about 0.1-0.9 mg/ml, about 0.1-0.8 mg/ml, about 0.1-0.7 mg/ml, about 0.1-0.5 mg/ml, about 0.5-10 mg/ml, about 0.6-10 mg/ml, about 0.7-10 mg/ml, about 0.8-10 mg/ml, about 0.9-10 mg/ml, about 1-10 mg/ml, about 2-10 mg/ml, about 3-10 mg/ml, about 4-10 mg/ml, about 5-10 mg/ml, about 6-10 mg/ml, about 7-10 mg/ml, about 8-10 mg/ml, about 9-10 mg/ml.

In some embodiments, a laminin is at a concentration of about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml. In some embodiments, a laminin is at a concentration of about 1 mg/ml.

### Physical Characteristics of Biocompatible Constructs

A biocompatible construct disclosed herein can have one or more characteristics described herein and/or can be part of a system disclosed herein. In some embodiments, a biocompatible construct disclosed herein is a linear construct.

In some embodiments, a system comprising a biocompatible construct disclosed herein has an outer diameter of about 0.07-70 mm, about 0.1-70 mm, 0.2-70 mm, about 0.3-70 mm, about 0.4-70 mm, about 0.5-70 mm, about 0.6-70 mm, about 0.7-70 mm, about 0.07-60 mm, about 0.07-50 mm, about 0.07-40 mm, about 0.07-30 mm, about 0.07-20 mm, about 0.07-10 mm, about 0.07-5 mm, about 0.07-1 mm, about 0.07-0.5 mm, about 0.07-0.7 mm, about 0.1-1 mm, about 0.2-5 mm, about 0.3-1 mm, about 0.4-0.9 mm, or about 0.5-0.8 mm. In some embodiments, a system comprising a biocompatible construct disclosed herein has an outer diameter of about 0.07 mm, about 0.1 mm, about 0.2 mm, about 0.3 mm, about 0.4 mm, about 0.5 mm, about 0.6 mm, about 0.7 mm, about 1 mm, about 5 mm, about 10 mm, about 20 mm, about 30 mm, about 40 mm, about 50 mm, about 60 mm, or about 70 mm. In some embodiments, a system comprising a biocompatible construct disclosed herein has an outer diameter of about 0.7 mm.

In some embodiments, a system comprising a biocompatible construct disclosed herein has an inner diameter of about 0.03-30 mm, about 0.05-30 mm, about 0.1-30 mm, about 0.15-30 mm, about 0.2-30 mm, about 0.25-30 mm, about 0.3-30 mm, about 0.03-25 mm, about 0.03-20 mm, about 0.03-15 mm, about 0.03-10 mm, about 0.03-5 mm, about 0.03-1 mm, about 0.03-0.5 mm, or about 0.03-0.3 mm. In some embodiments, a system comprising a biocompatible construct disclosed herein has an inner diameter of about 0.03 mm, about 0.05 mm, about 0.1 mm, about 0.2 mm, about 0.3 mm, about 0.5 mm, about 1 mm, about 5 mm, about 10 mm, about 15 mm, about 20 mm, about 25 mm or about 30 mm. In some embodiments, a system comprising a biocompatible construct disclosed herein has an inner diameter of about 0.3 mm.

### Methods of Using Systems Disclosed Herein

A system disclosed herein comprising a plurality of astrocytes obtained from a GDMSC can be used, e.g., as an in vitro test bed; for neural tissue engineering approaches; to promote differentiation, proliferation and/or migration of cells; and/or to treat or ameliorate a symptom of one or more neuropathologies (e.g., as described herein).

In some embodiments, a system disclosed herein is used in a method of promoting cell migration, differentiation and/or proliferation, comprising providing a system comprising: (a) a plurality of astrocytes derived from at least one gingiva-derived mesenchymal stem cell; and (b) a biocompatible construct comprising a first end, a second end and a body comprising a luminal core.

In some embodiments, a method promotes migration, differentiation and/or proliferation of a cell endogenous to a subject in which a biocompatible construct is implanted. In some embodiments, an endogenous cell comprises a neural precursor cell, a neuroblast, a neuron, a progenitor cell, a glial cell, an astrocyte, and/or an endothelial cell.

In some embodiments, a method promotes migration of one or more cells provided in a system; or one or more cells derived from, differentiated from or a progenitor of a cell provided in a system.

In some embodiments, migration of one or more cells occurs within a system, throughout a system, out of a system or into a system.

In some embodiments, a system disclosed herein is used in a method of treating and/or ameliorating one or more symptoms of a neurodegenerative disorder or a neurological disorder in a subject, comprising providing to the subject a system comprising: (a) a plurality of astrocytes derived from at least one gingiva-derived mesenchymal stem cell (GDMSC); and (b) a biocompatible construct comprising a matrix and having a first end, a second end and a body.

In some embodiments, a neurodegenerative disorder comprises a disorder with injury or degeneration to one or more neurons.

In some embodiments, a neurodegenerative disorder comprises brain injury or spinal cord injury.

In some embodiments, brain injury comprises acute brain injury, degenerative brain injury, traumatic brain injury (TBI), or chronic brain injury.

In some embodiments, a neurological disorder comprises: Parkinson's, Alzheimer's, Huntington's, prion disease, motor neuron disease, spinocerebellar ataxia, spinal muscular atrophy, amyotrophic lateral sclerosis (ALS), encephalitis, epilepsy, head and brain malformations, or hydrocephalus.

In some embodiments, a subject is a human.

In some embodiments, of any of the methods disclosed herein, a system is implanted into a subject. In some embodiments, a system is implanted into the brain of a subject. In some embodiments, a system is implanted: (i) at, near or within a brain lesion in a subject; (ii) at, near or within an area in the brain with insufficient neurons or neuronal connections, or with damaged neurons or neuronal connections; (iii) at, near or within a subventricular zone, an endogenous rostral migratory stream or a neurogenic niche; and/or (iv) at, near or within a region of a brain affected by a brain injury, a neurodegenerative disease or disorder, or a neurodevelopmental disease or disorder.

In some embodiments, a system is characterized in that when implanted into an organism it ameliorates or reduces severity of one or more symptoms of a disorder.

In some embodiments, a system is characterized in that when implanted into an organism it promotes migration of one or more cells.

In some embodiments, migration of one or more cells is compared to a comparator. In some embodiments, a comparator comprises an organism implanted with an otherwise similar system without a plurality of astrocytes; or without a plurality of astrocytes derived from at least one GDMSC.

In some embodiments, one or more migrating cells comprises one or more endogenous host cells. In some embodiments, an endogenous host cell comprises a neural precursor cell, a neuroblast, a neuron, a progenitor cell, a glial cell, an astrocyte, and/or an endothelial cell.

In some embodiments, one or more migrating cells comprises: one or more cells provided in a system; or one or more cells derived from, differentiated from or a progenitor of a cell provided in a system. In some embodiments, migration of one or more cells occurs within a system, throughout a system, out of a system or into a system.

In some embodiments, migration of one or more cells occurs to a site of implantation, or away from a site of implantation.

In some embodiments, a method disclosed herein is characterized in that when implanted in an organism, it promotes differentiation of one or more cells, e.g., an endogenous cell in which a system is implanted, or a cell provided in or derived from a system disclosed herein.

In some embodiments, a method disclosed herein is characterized in that when implanted in an organism, it promotes proliferation of one or more cells, e.g., an endogenous cell in which a system is implanted, or a cell provided in or derived from a system disclosed herein.

### EXEMPLIFICATION

### Example 1: An Implantable Human Stem Cell-Derived Tissue-Engineered Rostral Migratory Stream for Directed Neuronal Replacement

The rostral migratory stream (RMS) facilitates neuroblast migration from the subventricular zone to the olfactory bulb throughout adulthood. Brain lesions attract neuroblast migration out of the RMS, but resultant regeneration is insufficient. Increasing neuroblast migration into lesions has improved recovery in rodent studies. Techniques for fabricating an astrocyte-based Tissue-Engineered RMS (TE-RMS) intended to redirect endogenous neuroblasts into distal brain lesions for sustained neuronal replacement were previously developed. This Example demonstrates that astrocytes can be derived from adult human gingiva mesenchymal stem cells and used for TE-RMS fabrication. This Example also shows that key proteins enriched in the RMS are enriched in TE-RMSs. Furthermore, the human TE-RMS facilitated directed migration of immature neurons in vitro. Finally, human TE-RMSs implanted in athymic rat brains redirect migration of neuroblasts out of the endogenous RMS. By emulating the brain's most efficient means for directing neuroblast migration, in some embodiments, a TE-RMS offers a promising new approach to neuroregenerative medicine.

### INTRODUCTION

Adult neurogenesis continues in the mammalian brain in the subgranular zone of the dentate gyrus and the subventricular zone (SVZ) surrounding the lateral ventricles^{1,2}. Neural precursor cells (NPCs) in the SVZ can differentiate into neuroblasts and migrate through the rostral migratory stream (RMS) to the olfactory bulb (OB) where they mature into interneurons and integrate into existing circuitry³⁻⁶. Neuroblasts migrate in chain formation at a rate between 30-70 μm/hour (0.72-1.68 mm/day)⁷⁻¹⁰ along astrocytes that comprise the RMS (FIG. 1B). Various directional cues guide SVZ neuroblasts on their journey through the RMS, critical in regulating rapid and unidirectional neuroblast migration^{3,11,12}. For example, the diffusible protein Slit1 is released by migrating neuroblasts and its corresponding Robo2 receptor is expressed on RMS astrocytes^{3,11,13}. Via Slit1 release, migrating neuroblasts tunnel through the astrocytes of the RMS through a chemorepellent interaction with astrocytic Robo2 receptors, forming the glial tube that enables proper migration through the RMS^{11,14}. Additionally, the membrane-cytoskeletal linking protein ezrin is expressed at high levels in RMS astrocytes, hypothesized to regulate migration via two-way communication with migrating neuroblasts¹⁵.

Endogenous neurogenesis is upregulated in the SVZ following brain injury¹⁶⁻¹⁸. Increased neurogenesis has been reported in the rodent SVZ following multiple experimental models of acquired brain injury, including but not limited to stroke¹⁹⁻²⁸, controlled cortical impact brain injury²⁹⁻³¹, and lateral fluid percussion brain injury^{32,33}. Following brain injury, these newly formed NPCs can mature into neuroblasts, divert from the SVZ/RMS, and migrate toward injured brain regions^{27,28,34,35} (FIG. 1C). However, the quantity of SVZ-derived cells that mature into functional neurons in injured regions appears insufficient to improve functional recovery at physiological levels^{26,36}. There is a plethora of preclinical research demonstrating that enhancing the redirection of neuroblasts from the SVZ into regions of injury with experimental intervention can induce functional recovery following injury^{14,21,37-48}. For example, overexpression of Slit1 in neuroblasts enhanced SVZ neuroblast migration into a stroke-induced lesion, maturation into striatal neurons, integration into circuitry, and improved functional recovery following experimental stroke in rodents¹⁴.

Neural tissue engineering has introduced the possibility of developing customized therapies to enhance neuronal regeneration following traumatic brain injury. A variety of biomaterial and tissue engineering technologies have been developed to enhance the neurogenic potential of the SVZ and redirect the migration of SVZ neuroblasts to neuron-deficient brain regions following various experimental brain injuries (for recent review, see Purvis et al., 2020⁴⁹). The evidence from tissue engineering techniques, along with that of pharmacological and genetic approaches, collectively demonstrates that experimental intervention to enhance the brain's intrinsic repair mechanism to replace lost or damaged neurons with endogenous SVZ NPCs can improve recovery after acquired brain injury. However, while promising, these interventions have thus far only afforded transient re-direction of neuroblasts, while in some embodiments, a sustained influx of new neurons is likely required for functional improvements across a spectrum of brain injury severities.

Three-dimensional tissue engineered “living scaffolds” that replicate specific neuroanatomical features of neural architecture and/or circuitry have been previously developed. Implantation of these fully formed, living microtissue scaffolds in vivo has allowed successful facilitation of nervous system repair by replacing and/or augmenting lost circuitry⁵⁰⁻⁵⁴ and facilitating axonal regeneration and pathfinding⁵⁵. In addition, the first Tissue-Engineered Rostral Migratory Stream (TE-RMS) was recently developed, which is an implantable scaffold designed to replicate the endogenous RMS⁵⁶⁻⁵⁸. This engineered neuronal replacement strategy replicates the only known mechanism for continual, long-distance neuroblast redirection that occurs intrinsically within the adult brain via the RMS. By recapitulating the structure and function of the glial tube at the core of the RMS, in some embodiment, a TE-RMS is designed to promote sustained delivery of neuroblasts to neuron-deficient regions following injury or neurodegenerative disease. In some embodiments, it is expected that stable, long-term neuroblast redirection via this engineered living scaffold will set this technology apart from previous strategies that have typically induced transient neuronal redirection.

The TE-RMS is biofabricated within a small-diameter agarose microcolumn that promotes astrocyte bundling with extracellular matrix and self-assembly into long, longitudinally-aligned cables (FIG. 1D). Previous experiments to date have demonstrated that the basic structure of the TE-RMS (tight astrocytic bundles with bidirectional morphology) recapitulate the cell type and basic morphology of the endogenous RMS^{56,57}. It was hypothesized that the living astrocytes of the TE-RMS will also engage in dynamic, two-way communication with neuroblasts as they migrate through the scaffold (FIG. 1E), made possible by emulating the specific protein expression that facilitates neuroblast migration within the endogenous RMS. Here, the TE-RMS has the potential to serve as an anatomically-relevant test bed to study the interaction between neuroblasts and the RMS in vitro. One of the uses for the TE-RMS is to enable redirection of endogenous neuroblasts from the SVZ/RMS to neuron-deficient brain regions in vivo. Following focal brain injury (FIG. 1C), the TE-RMS could be implanted into the brain spanning from the SVZ/RMS into the injured brain region (FIG. 1F). In some embodiments, it is hypothesized that neuroblasts will divert from the SVZ/RMS and migrate in chain formation through the TE-RMS and into the lesion (FIG. 1G). In some embodiments, future studies can test whether gradual, sustained introduction further promotes neuroblast survival and maturation following arrival at their new location (FIG. 1H), thereby repopulating injured regions.

In the current study, protein expression in astrocytes of the TE-RMS was compared to that of the glial tube in the endogenous RMS. This Example also reports the ability to fabricate the TE-RMS from a readily available source of adult human gingiva mesenchymal stem cells (GMSCs) from which astrocytes can be derived within one week using non-genetic techniques without the need for dedifferentiation. This enhances the translational potential of this technology by introducing the possibility that, in some embodiments, with further development, human autologous TE-RMS implants can be created. The data herein also demonstrates that the human TE-RMS facilitates immature neuronal migration in vitro. Finally, it is reported that implantation of the human TE-RMS into the athymic rat brain facilitates migration of endogenous neuroblasts out of the native RMS and throughout the TE-RMS, providing surgical feasibility and proof-of-concept evidence for this technology.

### Results

### Astrocytes of the Endogenous Rat RMS are Enriched in Robo2 and Ezrin

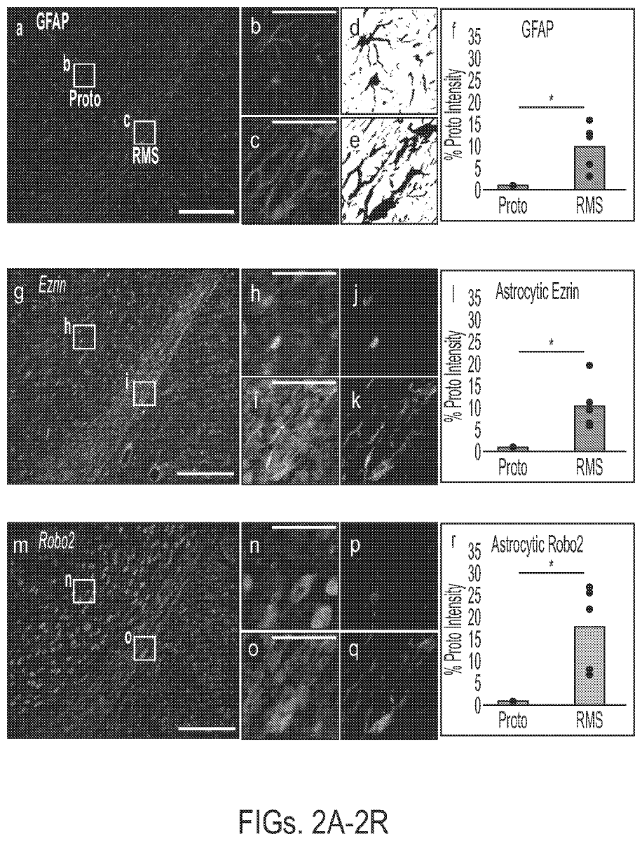

Previous studies have characterized the enrichment of protein markers in the glial tube astrocytes of the RMS as compared to surrounding protoplasmic astrocytes. As such, the expression and distribution of these enriched proteins was compared in astrocytes of the native RMS versus astrocytes of the TE-RMS. Fluorescence immunohistochemistry (IHC) was applied in sagittally-sectioned FFPE adult rat brains (n=5 brains) to label GFAP, Ezrin, and Robo2 (FIGS. 2A-2R. Images of each individual label in the RMS and surrounding tissue was captured via epifluorescence microscopy. Standardized ROIs containing glial tube astrocytes of the RMS and protoplasmic astrocytes from the surrounding area were used for pairwise comparisons of labeling intensities. Since these proteins can be expressed by other cell types, the GFAP channel was used to spatially isolate astrocytic signal from each channel for quantification. Mean intensities were calculated for each ROI to remove the influence of differences in astrocytic area. Comparing mean astrocytic intensities via two-tailed, paired Student's t-tests revealed that astrocytes of the RMS were significantly enriched in GFAP (FIG. 2F; t=3.770, df=4, p=0.02), Ezrin (FIG. 2L; t=3.642, df=4, p=0.02), and Robo2 (FIG. 2R; t=3.890, df=4, p=0.02), compared with surrounding protoplasmic astrocytes. It is also noted that the astrocytic intensity of GFAP, Ezrin, and Robo2 labeling in the endogenous rat RMS was higher than that of the surrounding protoplasmic astrocytes in every brain analyzed, though there was some variability in the size of those differences between brains.

### Astrocytes of the Rat TE-RMS are Enriched in Robo2 and Ezrin

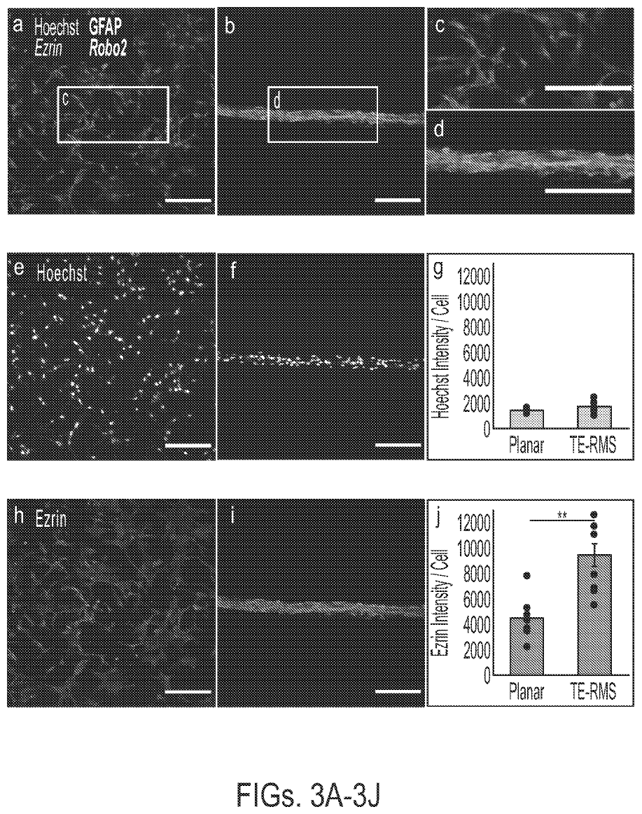

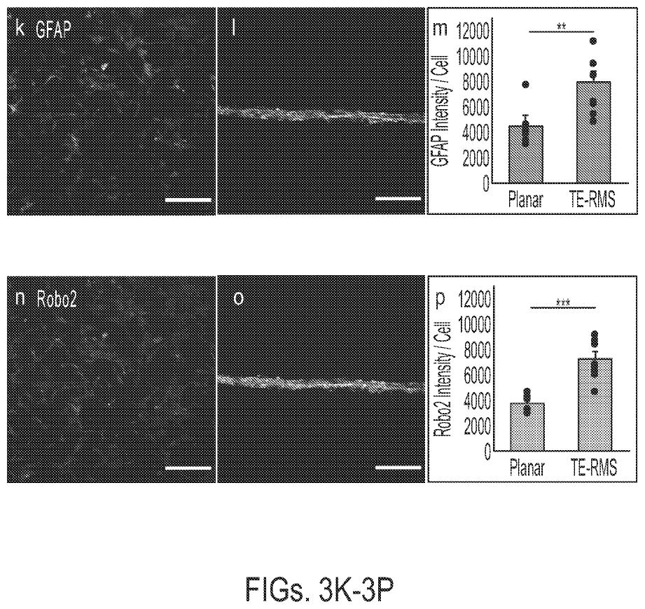

The development of fabrication techniques for the TE-RMS were previously reported, in which optimal microcolumn diameter, collagen concentration, media constituents, seeding density, and other factors were evaluated to facilitate astrocyte self-assembly into longitudinally-aligned, tightly bundled cords over a period of just 8 hours. Data on the similarities in the morphology and structural arrangement of rat TE-RMS astrocytes compared with astrocytes of the endogenous RMS were also previously disclosed⁵⁶⁻⁵⁸. In this Example, the hypothesis that TE-RMS astrocytes also recapitulate the enhanced expression of GFAP, Ezrin, and Robo2 observed in the endogenous RMS was evaluated (as verified in the experiments of FIG. 2). The enrichment of these proteins in the TE-RMS was tested by comparing fluorescence immunocytochemistry (ICC) labeling intensities in TE-RMS astrocytes with those of protoplasmic astrocytes from planar sister cultures imaged via epifluorescence microscopy (FIGS. 3A-3P). While seeding from the same sources provided excellent control for any unforeseen culture variability that could have interfered with the ability to test differences between planar vs. TE-RMS groups, the fabrication and culturing process after seeding led to the consideration of each sample as independent. Therefore, paired means testing was note used as in the brain sections of FIGS. 2A-2R, and instead two-tailed Student's t-tests were employed to compare labeling intensities in the planar cultures (n=6) and TE-RMSs (n=9) seeded from primary cortical rat astrocyte cultures. Due to the close proximity of cells in TE-RMSs as compared to planar sister cultures, Hoechst-stained nuclei were often in overlapping visual fields in TE-RMS images, making automated cell counting unreliable. Therefore, total nuclear area from the Hoechst channel for each image was measured to allow for unbiased, automated calculation of cell amount in each image for normalization. This revealed that Hoechst intensities of the planar and TE-RMS groups were not different (FIG. 3G). Also, as observed in the endogenous RMS, astrocytes of the TE-RMS were significantly enriched in Ezrin (FIG. 3J; t=3.845, df=13, p=0.002), GFAP (FIG. 3M; t=3.086, df=13, p=0.009), and Robo2 (FIG. 3P; t=4.855, df=13, p=0.0003), as compared with the astrocytes in the planar sister cultures.

Astrocytes can be Derived from Adult Human GMSCs and Used for TE-RMS Fabrication

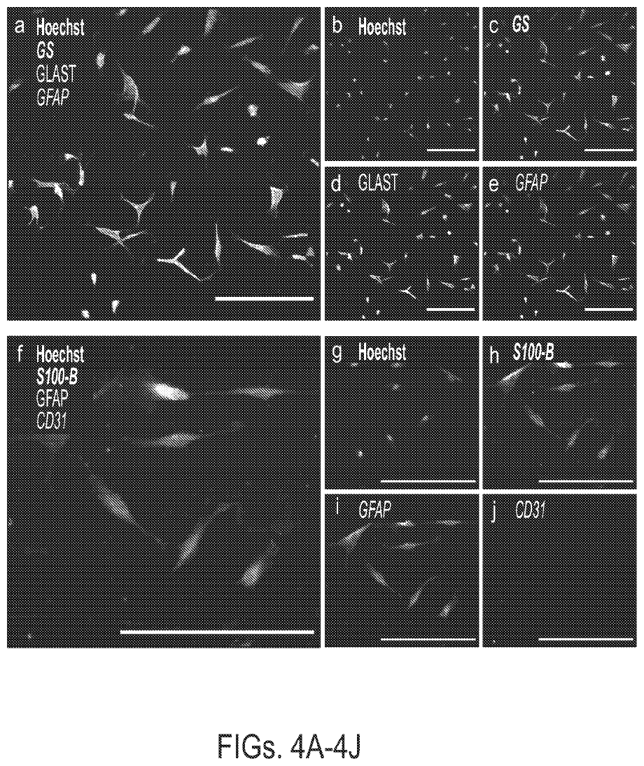

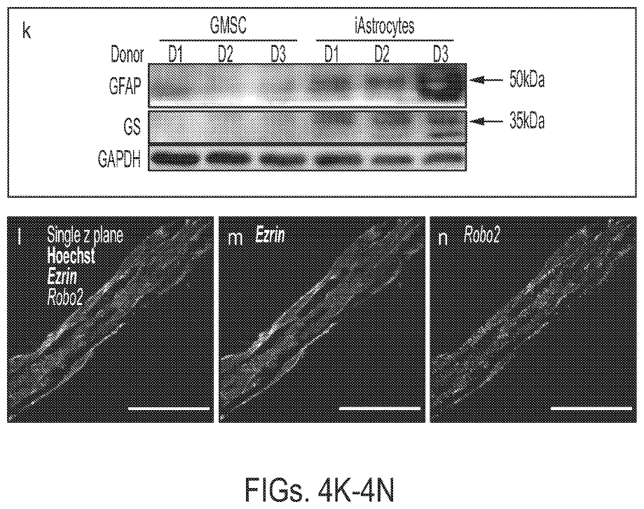

To identify a clinically relevant starting biomass for TE-RMS fabrication, the efficacy of a novel differentiation protocol to derive astrocytes from GMSCs was evaluated. This protocol was adapted from a previously published method for astrocyte derivation from oral mucosal stem cells⁵⁹. Here, this non-genetic derivation protocol was successful applied to GMSCs from three deidentified adult human patients obtained via minimally invasive punch biopsy. After the derivation process—which takes less than a week—the cultured cells from each subject expressed astrocytic proteins Glutamine Synthetase (GS), Glutamate Aspartate Transporter (GLAST), GFAP, and S100-β and were negative for the endothelial marker CD31 (FIGS. 4A-J). Western blot analyses confirmed that GMSCs from three de-identified donors did not express GFAP or GS prior to derivation, but the astrocytes derived from GMSCs expressed GFAP and GS (FIG. 4K). The morphology of these cells was also consistent with astrocytes in planar culture, and they thrived under astrocytic culture conditions. These cells were also compatible with passaging techniques for astrocyte culture purification, including vigorous mechanical perturbation prior to trypsinization that is commonly applied to detach non-astrocytic cells from culture flasks for removal prior to passaging. Furthermore, when the human GMSC-derived astrocytes were used as a starting biomass for TE-RMS fabrication, they rapidly self-assembled into cables of longitudinally-aligned, bidirectional astrocytes within the same 8 hour timeframe observed when fabricating with primary astrocytes from rat cortex. This rapid remodeling/bundling appears to be unique to astrocytes, as when the same fabrication methods were applied using Schwann cells, bundling and alignment took several days⁶⁰. These human TE-RMSs stained positive for Ezrin and Robo2, which can be seen localized to the plasma membrane in the single high magnification z plane confocal images of FIGS. 4L-4N.

### Astrocytes of the Human TE-RMS are Enriched in Robo2 and Ezrin

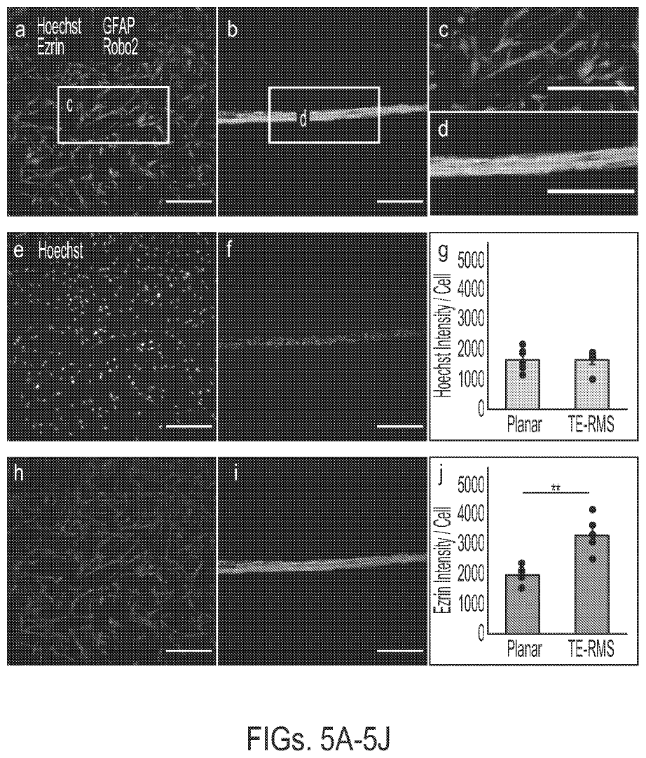

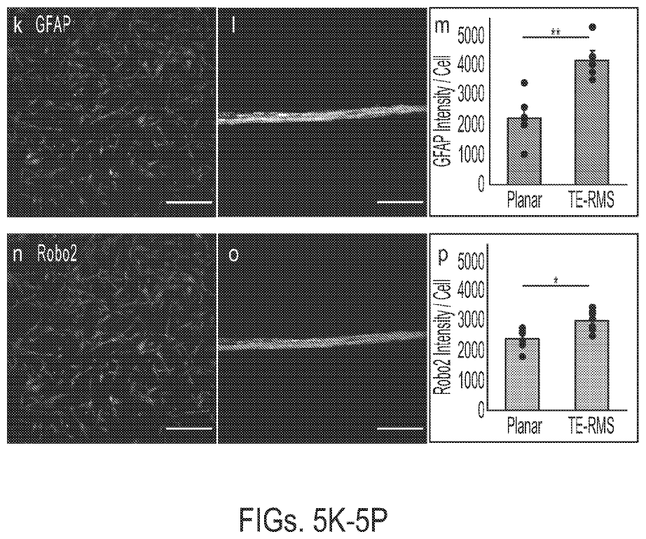

Fluorescence ICC with laser confocal microscopy was used to confirm that TE-RMSs fabricated from human GMSC-derived astrocytes expressed GFAP, Ezrin, and Robo2 (FIGS. 4F-4K), the combination of which is characteristic of glial tube astrocytes of the RMS. Then, to test whether the human GMSC-derived TE-RMS is enriched in GFAP, Ezrin, and Robo2 as observed in the endogenous rat RMS and the cortical rat astrocyte TE-RMS, the same experimental techniques and analyses used to investigate the rat TE-RMS were employed (see FIGS. 3A-3P). Hoechst intensities of the planar (n=6) and TE-RMS (n=5) groups were not different (FIG. 5G). As observed in the endogenous RMS and rat TE-RMS, human GMSC-derived TE-RMSs were significantly enriched in Ezrin (FIG. 5J; t=4.720, df=9, p=0.001), GFAP (FIG. 5M; t=4.350, df=9, p=0.002), and Robo2 (FIG. 5P; t=2.639, df-9, p=0.027), compared with the astrocytes of their planar sister cultures.

### The Human TE-RMS Facilitates Directed Migration of Immature Rat Neurons In Vitro

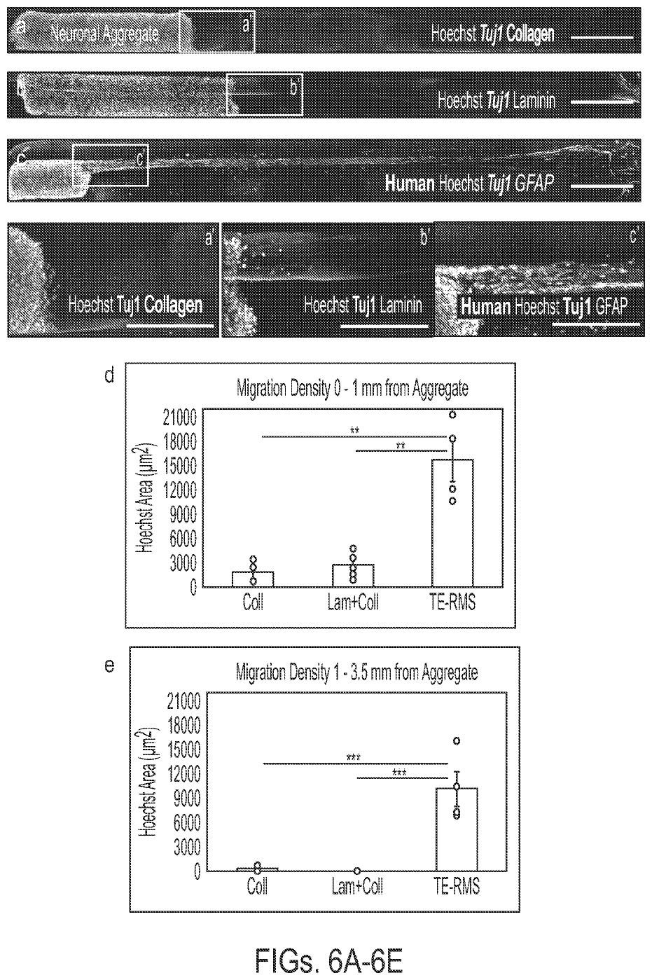

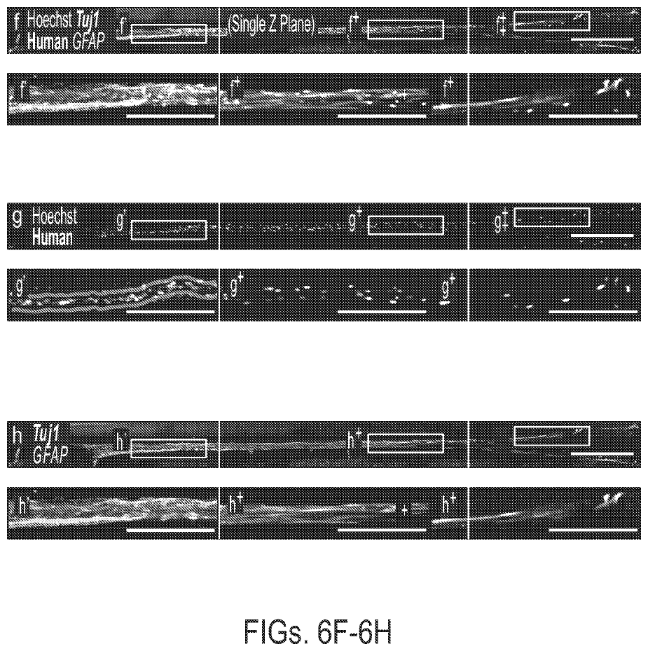

Looking beyond replicating the morphology, arrangement, and protein expression of the endogenous RMS, in some embodiments, a human TE-RMS is expected to have a substantially similar function as an endogenous RMS to be used for clinical application or as an in vitro test bed. Techniques from a previously reported migration assay in the rat TE-RMS were adapted⁵⁸, and migration of immature rat cortical neurons through the human TE-RMS was assessed as compared to acellular collagen-coated or collagen+laminin-coated control columns (FIGS. 6A-6H). Immature cortical neuronal aggregates were placed at one end of a microcolumn containing a fully formed human TE-RMS, an acellular collagen control, or an acellular collagen+laminin control, and migration from the aggregate to the opposite side of the microcolumn was assessed at 72 hours. For ICC analyses, Hoechst nuclear stain was applied and immunostaining for Tuj1 (Beta-III-tubulin) was performed to label neuronal processes. Co-staining for Human Nuclei and GFAP in the TE-RMSs, collagen in the collagen controls, and laminin in the collagen+laminin controls was also performed. Under these conditions, little if any migration into the acellular collagen control columns (n=4) or into the acellular collagen+laminin columns (n=5) was observed, while immature rat neurons (Hoechst-positive/Human-negative nuclei with Tuj1-positive processes) migrated through the entire 4 mm length of human TE-RMS within 72 hours (n=4). Neuronal aggregates exhibited notably different behavior when seeded into the collagen+laminin control columns, in which they exhibited little migration out of the aggregate but instead extended neurites into the ECM to an average length of 353.6 μm (SEM=71.0) at 72 hours (FIG. 6B′). Neuronal aggregates did not exhibit any measurable neurite extension in the collagen-only control columns. To compare migration quantitatively, the area of Hoechst-positive, Human-negative nuclei (nuclei from the immature neuronal aggregate) was measured in a zone proximal to the aggregate (within 1 mm) and a zone more distal (1-3.5 mm from aggregate). The amount of migrating neurons within 1 mm of the aggregate (FIG. 6D) in the TE-RMS was significantly greater than in the acellular collagen (t=5.223, df=10, p=0.001) or collagen+laminin controls (t=4.460, df=10, p=0.004). Beyond 1 mm there were essentially no migrating neurons in the controls whereas migrating neurons were found throughout the entire length of the TE-RMSs, and accordingly the amount of migrating neurons between 1-3.5 mm of the aggregate (FIG. 6E) in the TE-RMS was significantly greater than in the acellular collagen (t=5.626, df=10, p=0.0007) or collagen+laminin controls (t=6.375, df=10, p=0.0002). A single z plane view of neuronal migration through the TE-RMS (FIGS. 6F-H^{‡}) allowed for the visualization of cell-to-cell interactions in greater detail, though in some cases components were out of plane (e.g. nuclear signal with processes out of the visible z plane). Completing a 4 mm journey through the TE-RMS construct within 72 hours indicated an average migration rate of at least 56 μm/hour, placing them within the reported range of 30-70 μm/hour for neuroblast migration in the endogenous RMS7.10.61. Hoechst-positive/Human-negative nuclei from the rat cortical aggregate were densest near the aggregate, where a narrow “follow-the-leader” path could be most easily visualized (lines, FIG. 6G′). Hoechst-positive/Human-negative nuclei were also observed throughout the length of the human TE-RMS (white arrows, FIGS. 6G^{†} and G^{‡}). These migrating cells were Tuj1-positive, consistent with an immature neuronal phenotype (FIG. 6H), and their Tuj 1-positive processes ran parallel to—but did not overlap—the GFAP-positive processes of the human TE-RMS (FIGS. 6H′, 6H^{†} and 6H^{‡}).

The Human TE-RMS Redirects Migration of Neuroblasts from the Rat RMS In Vivo

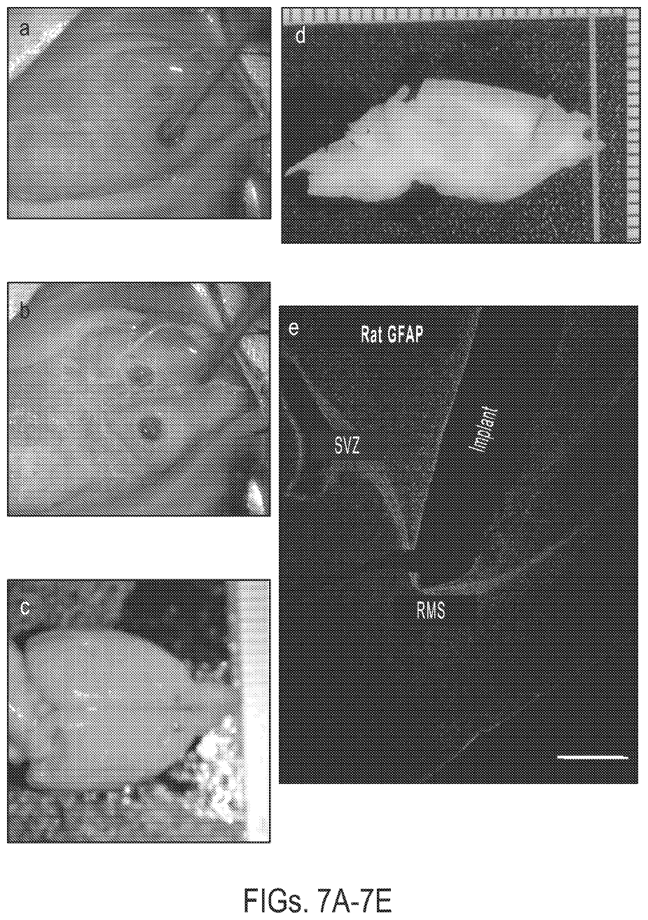

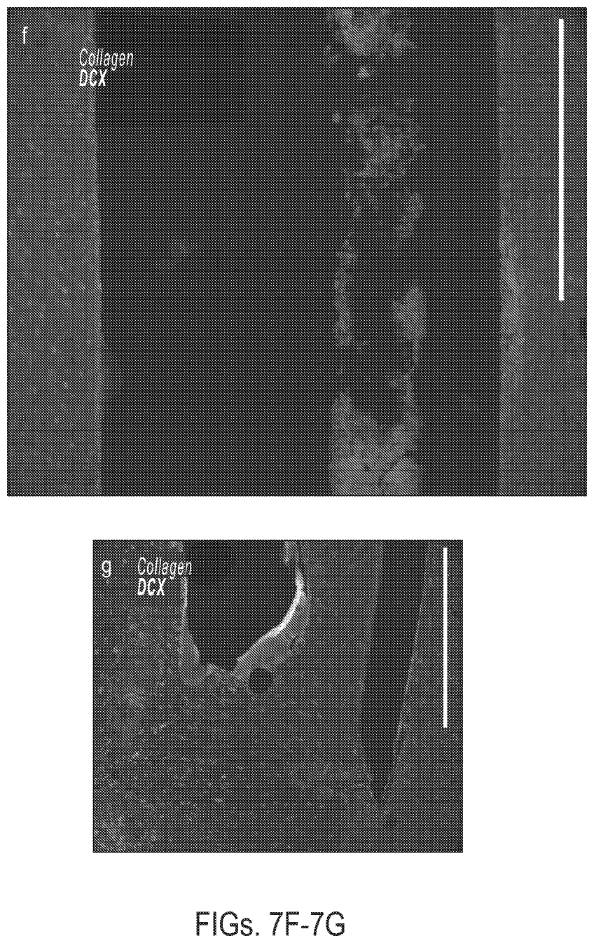

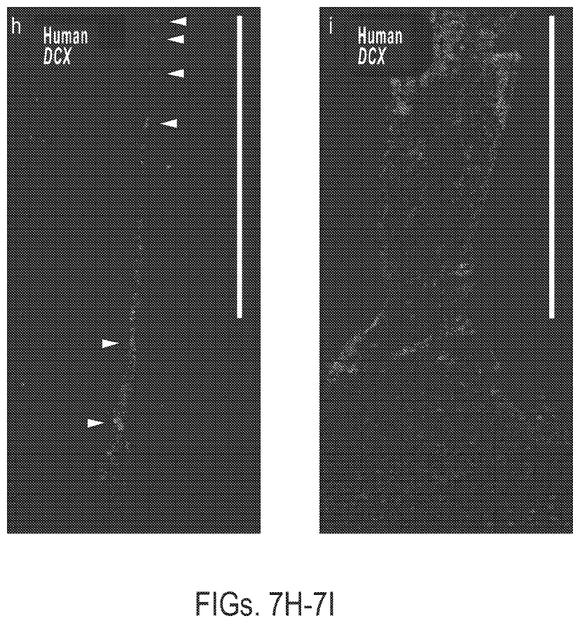

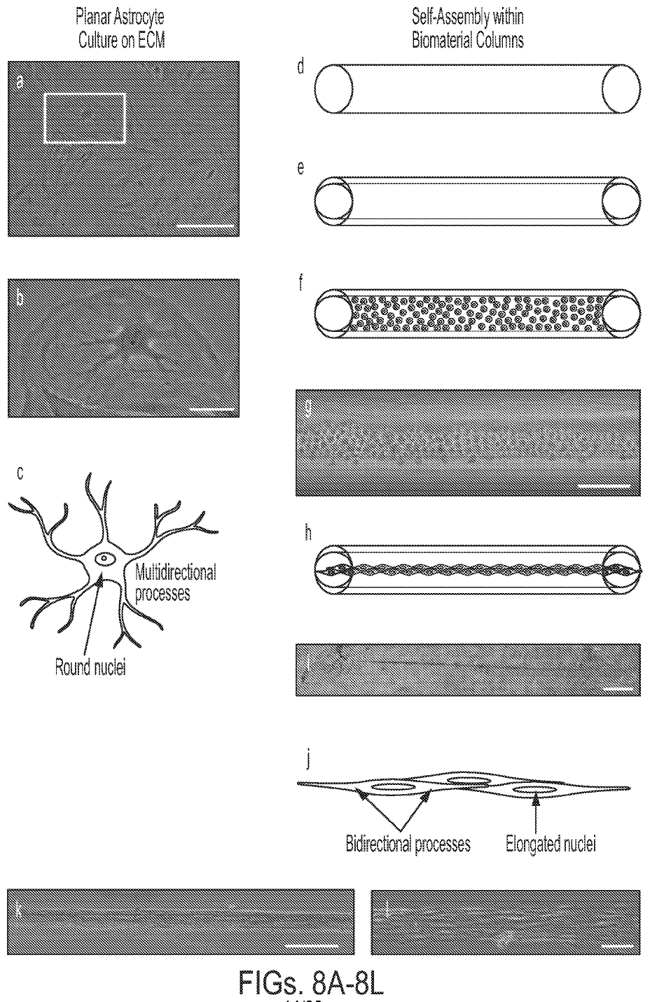

Finally, in vivo experiments with stereotactic implantation of human TE-RMSs into the brains of athymic rats were performed to test surgical feasibility and proof-of-principle for redirecting neuroblast migration away from the endogenous RMS (FIGS. 7A-7I). In athymic rats (n=6), pairs of 4 mm TE-RMSs and acellular collagen control microcolumns to span RMS to motor cortex were bilaterally implanted. Animals were euthanized six days later, and their FFPE brains were sagittally sectioned for IHC analyses. By injecting implants 2.5 mm rostral from bregma and 1 mm from midline in either direction at a depth of 5 mm, the endogenous RMS was reproducibly contacted as verified by gross pathology and epifluorescence microscopy (FIGS. 7D, 7E). During the six days following implantation, no alterations in behavior was observed and there was minimal disruption of surrounding areas by gross pathology. Doublecortin (DCX) positive cells were observed near the ends of the contralateral acellular collagen control implants, but were absent from central regions (FIGS. 7F, 7G). However, doublecortin-positive, Human-negative cells were observed-indicative of migrating endogenous rat neuroblasts-throughout the human TE-RMS implants (FIGS. 7H, 7I), suggesting that host cells were migrating through the TE-RMS while only incidental infiltration of host cells was taking place near the ends of the acellular control columns. Combined with the in vitro experiments of FIG. 6, this implantation study provides proof-of-principle evidence for redirection of neuroblast migration via the human GMSC-derived TE-RMS and surgical feasibility for its implantation into the brain to span the endogenous RMS and cortex.

The TE-RMS is the first biomimetic implantable microtissue designed to redirect the migration of endogenous neuroblasts out of the RMS and into distal lesions, intended to, in some embodiments, provide sustained delivery to replace lost neurons and improve functional recovery. In pursuit of this objective, the TE-RMS was developed to emulate the brain's only existing method for transporting neuroblasts to a distal area for neuronal replacement. Whereas prior studies have transplanted exogenous fetal grafts, single cell suspensions, or cells in 3-D matrices, the method disclosed herein is considerably different in that the living cytoarchitecture of the TE-RMS—mimicking the architecture the RMS—is fully biofabricated in vitro and then precisely delivered to unlock the regenerative potential of the brain's own endogenous neuroblasts. In the current study it was confirmed that the TE-RMS is enriched in Ezrin and Robo2, both of which are similarly enriched in the endogenous RMS and important for facilitating neuroblast migration. This Example also reports a new method for deriving astrocytes from adult human GMSCs adapted from a previous protocol applied to oral mucosa, potentially providing a minimally invasive autologous starting biomass for patients. Indeed, utilizing these cells for fabrication produces a human TE-RMS consisting of tightly-bundled, bidirectional, longitudinally-aligned, astrocytes enriched in GFAP, Ezrin, and Robo2. Furthermore, the human TE-RMS facilitated in vitro migration of immature neurons at rates within the range observed for migration of neuroblasts in the endogenous RMS. Finally, the data disclosed herein provides in vivo evidence of surgical feasibility for human TE-RMS implantation into athymic rat brains spanning from the RMS to the cortex, and proof-of-principle evidence that the human TE-RMS can redirect migration of endogenous rat neuroblasts out of the RMS and into cortex with no overt negative histological or behavioral effects on test subjects.

While the direct implantation of neural stem cells into injury sites has been shown to improve outcome by providing neurotrophic factors, and recent work has demonstrated that integration of human induced pluripotent stem cell (iPSC) derived neurons in rat cortex is possible⁶², appropriate maturation and integration of these exogenous cells to functionally replace lost neurons remains a significant challenge⁶³⁻⁶⁵. Furthermore, each delivery of exogenous neural stem cells requires invasive surgery, therefore these proposed treatment strategies typically involve only a single bolus delivery of exogenous stem cells. In contrast, a single surgical procedure to implant a TE-RMS could, in some embodiments, provide sustained delivery of endogenous NPCs by emulating the brain's own strategy for relocating and integrating new neurons. In addition, strategies for direct implantation of exogenous stem cells often rely on iPSCs that must be dedifferentiated from a more mature cell source, and therefore carry risks related to the retention of epigenetic memory from the original cell source (i.e. leading to de-differentiation)⁶⁶. In contrast, a TE-RMS provides the brain's own endogenous NPCs that do not require any prior de-differentiation, and therefore in some embodiments do not suffer from the phenotypic abnormalities sometimes associated with epigenetic memory in iPSCs. Genetic modification of neuroblasts may not represent a translational therapeutic strategy, but it does support the feasibility of enhancing neuroblast migration into lesions to facilitate neuroregeneration. A TE-RMS disclosed herein, in some embodiments, can be precisely implanted to span SVZ to lesion, providing a migratory pathway to augment and amplify this natural regenerative response of endogenous SVZ neuroblasts after injury without the need for genetic manipulation.

Increasing delivery of neuroblasts into lesions after injury has also been approached through pharmacological strategies and implantation of acellular permissive substrates^{49,67}. Pharmacological approaches have focused mainly on administration of neurotrophic factors. Intraventricular infusion of various combinations of neurotrophic factors including epidermal growth factor, erythropoietin, fibroblast growth factor, vascular endothelial growth factor, and others has been shown to improve short-term functional recovery by enhancing proliferation in the SVZ after injury, in turn increasing the number of neuroblasts migrating into lesions by virtue of increasing the overall number of neuroblasts³⁹. There is extensive evidence in humans and animal models for exercise-induced improvements in functional recovery after brain injury, and the effects are largely attributed to increased neuronal plasticity and proliferation of endogenous NPCs in response to exercise-induced increases in brain-derived neurotrophic factor^{68,69}. In the current study, in vitro chain migration of immature cortical neurons through a TE-RMS was observed, whereas columns loaded with ECM only (1 mg/mL collagen+1 mg/mL laminin) promoted neurite outgrowth of these immature cells and did not promote migratory behavior. The data disclosed herein is distinct from previous research demonstrating neuroblast migration along planar laminin and collagen in 2D culture in vitro (73). This difference in cell behavior is likely due to difference in immature neuronal phenotype (SVZ-derived versus cortical) as well as ECM preparation across in vitro studies leading to differential binding of immature neurons to laminin. Indeed, migration and maturation of neuroblasts along the RMS relies on complex, dynamic signaling between astrocytes and neuroblasts⁷⁴⁻⁷⁶ Unlike extracellular-matrix-based constructs that offer a permissive, acellular substrate^{70,72,73}, a TE-RMS disclosed herein possesses a unique, living astrocytic microtissue makeup that can provide directional, structural, and/or neurotrophic support, making it capable, e.g., of sending as well as responding to complex signals with migrating neuroblasts and the local micro-environment. Relying on an acellular substrate infused with a handful of signaling molecules is akin to trying to coordinate a complex project in a foreign language of which you speak only a few words and cannot hear or respond to anything anyone else is saying, versus the living microtissue TE-RMS that provides total fluency in the language and engagement in dynamic, collaborative conversations.

Glial tube astrocytes possess several features that make them unique among the widely heterogeneous astrocyte milieu. They possess a bidirectional morphology, extending processes in opposite directions along the glial tube in parallel with each other to form a cord-like bundle. It has been previously established that these structural features are recapitulated in the TE-RMS⁵⁶⁻⁵⁸. Glial tube astrocytes are also enriched in several proteins important for facilitating neuroblast migration. Ezrin—a member of the cytoskeleton-membrane linking ERM (Ezrin, radixin, moesin) protein family—is enriched in the astrocytes of the glial tubes, while its cousin radixin is enriched in migrating neuroblasts^{15,77}. In the endogenous RMS, astrocytic Robo2 receptors detect Slit1 protein released by neuroblasts, and this signaling results in tunnel formation in the astrocytic meshwork of the glial tubes to facilitate neuroblast migration⁷⁸. In this study, previous reports of enrichment of GFAP, Ezrin, and Robo2 in the glial tube astrocytes of the endogenous rat RMS were evaluated. Using the same methods, previous morphological and structural analyses were expanded to show that the TE-RMS—fabricated from either primary rat astrocytes or human GMSC-derived astrocytes—is also enriched in GFAP, Ezrin, and Robo2.

Building on this concept, in some embodiments, mimicking the RMS may also facilitate maturation of neuroblasts during migration along the TE-RMS, allowing the new neurons to functionally replace lost neurons after degeneration has occurred instead of merely acting as neurotrophic “factories” in the acute and sub-acute time periods. This strategy to enable replacement of lost neurons well after acquired brain injury or neurodegeneration is highly unique, and presents an innovative approach to repairing currently untreatable injuries affecting millions of patients. In some embodiments, their miniature form factor allows for minimally invasive, stereotactic delivery into the brain. In rat models, the ability to precisely microinject similar allogeneic neuronal microtissue constructs, which maintain their pre-transplant architecture, survive, and integrate into the native nervous system was previously disclosed^{52-54,79,80}, and TE-RMS implant experiments included in this study further demonstrate the surgical feasibility of this strategy.

In this study, the successful derivation of astrocytes from adult human GMSCs, their suitability as a starting biomass for TE-RMS fabrication, and proof-of-concept evidence that these human TE-RMSs facilitate neuroblast migration in vitro and in vivo is reported. Like iPSCs, the GMSCs offer minimally-invasive access to a patient-specific autologous starting biomass. However, the one-week GMSC-to-astrocyte derivation process takes only a fraction of the time of iPSC derivations, due in part to the fact that dedifferentiation is unnecessary in the case of GMSCs. That lack of a dedifferentiation step also means that GMSCs do not carry risks associated with epigenetic memory⁶⁶. The appeal of a patient-specific starting biomass is primarily due to avoiding the dangers of immune rejection.

In some embodiments, a TE-RMS strategy is intended, e.g., for neuronal replacement and functional regeneration. In some embodiments, a TE-RMS disclosed herein can be implanted after the acute period to allow the so-called “hostile” environment—marked by dysregulated interstitial tissue, ongoing cell death, and widespread inflammation—to dissipate. In some embodiments, this strategy is also intended to serve as a means for gradual, sustained delivery of neuroblasts into injury sites. In some embodiments, the potential spectrum of cell fates when redirecting neuroblasts to various discrete brain areas can be determined. In some embodiments, a TE-RMS can be used as a biofidelic test bed for performing efficient and precise mechanistic studies. A human TE-RMS disclosed herein has opened new avenues for potential study, and a promising novel approach to leveraging the endogenous regenerative potential of the brain.

### Methods

### Cell Culture

All procedures adhered to the National Institutes of Health Guide for the Care and Use of Laboratory Animals and were approved by the Institutional Animal Care and Use Committee at the University of Pennsylvania. For TE-RMS fabrication, primary astrocytes were harvested from the cortices of postnatal day 0-1 Sprague-Dawley rat pups (Charles River, Wilmington, MA). Cells were dissociated⁵⁷ and cultured in DMEM/F12 medium supplemented with 10% FBS and 1% Penicillin-Streptomycin in a 37° C./5% CO₂ cell culture incubator. Over several weeks, astrocyte cultures were maintained in tissue culture flasks and passaged at 80% confluency to purify the astrocyte population. Astrocytes between passages 4-10 were utilized for all in vitro experiments. For in vitro migration of immature neurons through the TE-RMS, primary rat neurons were dissociated from cortices of embryonic day 18 (E18) rats⁵⁷. Following tissue dissociation with trypsin-EDTA and DNAse I, a cell solution with a density of 1.0-2.0×10⁶ cells/mL was prepared. 12 μL of this solution was transferred to each well in the pyramidal micro-well array. The plate containing these micro-wells was centrifuged to produce cell aggregates.

### Fabrication of Hydrogel Micro-Columns

Hydrogel micro-columns composed of 3% agarose were utilized to induce the alignment of astrocytes and create the TE-RMS⁵⁷. Agarose was dissolved and heated in Dulbecco's phosphate-buffered saline (DPBS). An acupuncture needle (diameter=300 μm) was inserted into the bottom opening of a bulb dispenser. A glass capillary tube (inner diameter=701 μm) was inserted over the needle external to the bulb and secured to the rubber section of the bulb dispenser. Warm agarose was drawn into capillary tube with the needle in the center. After allowing the agarose to cool, the capillary tube was carefully removed, and the micro-columns were gently pushed off the needle into DPBS and sterilized by UV light for 30 minutes. Micro-columns had an outer diameter of 701 μm and an inner diameter of 300 μm. Optimal micro-column dimensions for inducing alignment and bundling of astrocytes were determined based on previous experiments⁵⁶.

### Fabrication of Tissue-Engineered Rostral Migratory Streams

Hydrogel micro-columns were cut with angled forceps to a length of 4 mm. The inner lumen of the micro-columns was loaded with 1 mg/mL rat tail type 1 collagen diluted in cell culture medium. The collagen-loaded constructs were incubated for 3 hours at 37° C./5% CO₂ to allow for collagen polymerization and dehydration yielding in a hollow microcolumn with the surface of the inner lumen coated in collagen. After the complete collagen polymerization, the inner lumen of the micro-columns was seeded with astrocytes in serum-free co-culture media at a density of ˜1 million cells/mL (optimal seeding density confirmed by Winter et al., 2016⁵⁶). Co-culture media consisting of Neurobasal medium supplemented with 2% B-27, 1% G-5, 0.25% L-Glutamine, and 1% Penicillin-Streptomycin induced the astrocytes into a process-bearing phenotype. Columns were seeded twice with astrocytes to ensure that the entire interior of each micro-column was filled with cells. Following astrocyte seeding, columns were incubated at 37° C./5% CO₂ for one hour and subsequently reinforced with 1 mg/mL collagen. Collagen reinforcement provided more ECM to the astrocytes, helping to prevent collapse during astrocyte bundling. Following reinforcement, astrocyte-loaded columns were incubated for another hour at 37° C./5% CO₂, flooded with warm co-culture media, and returned to the cell culture incubator. Over a relatively short time period of ˜8 hours, the astrocytes extend processes to gather collagen and self-assemble into a bundled cord of longitudinally-aligned astrocytes with bidirectional processes, effectively forming TE-RMSs. For experimental purposes, TE-RMSs were utilized 24 hours after astrocyte seeding. Acellular collagen control columns for in vitro migration assays were prepared as above but with no addition of cells. For acellular collagen/laminin columns, a mixture of 1 mg/mL collagen and 1 mg/mL mouse laminin diluted in cell culture medium was loaded into micro-columns. Following extracellular matrix polymerization at 37° C./5% CO2 (approximately 3 hours), columns were flooded with warm co-culture media and returned to the cell culture incubator.

TE-RMS Extraction from Microcolumns for Immunocytochemistry

Following overnight bundling of astrocytes and formation of the TE-RMS, astrocytic bundles were extracted from hydrogel micro-columns onto glass coverslips using surgical forceps and a stereoscope for visual guidance. TE-RMSs were slowly drawn out of the micro-columns into a bead of collagen diluted in culture media and left to dry for 15 minutes at 37° C./5% CO₂ to facilitate coverslip adhesion prior to fixation. Extraction was not performed with migration assay columns as it would disrupt the neuronal aggregate, and they were instead fixed, stained, and imaged within the microcolumns to keep the assay system intact.

Astrocyte Derivation from Adult Human GMSCs

Healthy human gingival tissues were obtained as remnants of discarded tissues under the approved Institutional Review Board (IRB) protocol at University of Pennsylvania. All procedures and methods were carried out in accordance with relevant guidelines and regulations. Informed consents were obtained from all participating human subjects for the collection of fresh tissues. Mesenchymal stem cells isolated from de-identified human gingiva were run through a non-genetic process similar to a method previously applied to derive astrocytes from human oral mucosal stem cells⁵⁹. The derivation takes less than a week, and was accomplished in collaboration with the Le Laboratory at the University of Pennsylvania, with whom techniques for deriving neural crest stem cells and Schwann cell-like cells from GMSCs were previously developed⁸²⁻⁸⁴ The derivation began with 72 hour incubation in serum-free low-glucose DMEM with 100 μg/ml streptomycin, 100 U/ml penicillin, 1250 U/ml Nystatin, and 2 mM glutamine supplemented with 20 ng/ml N2 supplement (Thermo Scientific), basic fibroblast growth factor 2 (PeproTech, Rocky Hill, NJ), and epidermal growth factor (PeproTech). After 72 hours the media was replaced with serum-free low-glucose DMEM with 100 μg/ml streptomycin, 100 U/ml penicillin, 1250 U/ml Nystatin, and 2 mM glutamine supplemented with 1 mM dibutyryl cyclic AMP (Sigma-Aldrich), 0.5 mM 3-isobutyl-1-methylxanthine (Sigma-Aldrich), 50 ng/ml Neuregulin (PeproTech), and 1 ng/ml platelet-derived growth factor (PeproTech). After derivation, astrocytes were cultured in DMEM/F12 medium supplemented with 10% FBS and 1% Penicillin-Streptomycin and passaged at 80% confluency with mechanical perturbation prior to trypsinization to purify the astrocyte population. Cells between passages 4-10 were utilized for all in vitro and in vivo experiments.

### In Vitro Migration Assay

Following overnight bundling of astrocytes and formation of human GMSC-derived TE-RMSs, immature rat cortical neuronal aggregates were placed into one end of the micro-columns using surgical forceps and a stereoscope for visual guidance. Aggregates were placed such that they contacted one end of the fully formed TE-RMS within the column and placed in the same relative position in the acellular collagen and collagen/laminin columns. Columns loaded with aggregates were returned to the 37° C./5% CO₂ cell culture incubator and were fixed 72 hours following neuronal aggregate seeding.

### In Vivo Implantation

Athymic (immunodeficient) adult male rats (Strain RNU 316 homozygous; Charles River, Wilmington, MA) were maintained under isoflurane anesthesia and mounted in a stereotaxic frame. Subjects were subcutaneously administered bupivacaine (2.0 mg/kg) for local analgesia. Bilateral craniotomies were performed +2.5 mm anterior to bregma and +1 mm from midline on either side. Immediately prior to implantation, a 4 mm human GMSC-derived TE-RMS or acellular collagen control microcolumn was drawn into a thin-walled 21XX-gauge (813 μm outer diameter, 737 μm inner diameter) needle in warm DPBS. The needle was centered over the craniotomy and lowered at a rate of 2 mm/min. The column was delivered into the brain, and the needle was slowly retracted at a rate of 1 mm/min while the plunger was fixed in place to deliver the column without expulsive force. Subjects (n=6) were implanted bilaterally with a human GMSC-derived TE-RMS construct in the right hemisphere and an acellular collagen control microcolumn in the left. Stereotaxic coordinates (AP+2.5; ML+1; DV−5 mm relative to bregma) provided consistent implantation of all columns spanning from the RMS to the cortex. Following column delivery, the craniotomies were covered with a thin sterile PDMS disc and bone wax and the scalp was sutured. Subjects were administered slow-release meloxicam (4.0 mg/kg) for sustained post-surgical analgesia.

### Immunocytochemistry and Immunohistochemistry

The endogenous RMS was analyzed via immunohistochemistry in sagittal brain sections from 5 archival formalin-fixed paraffin-embedded (FFPE) adult Sprague Dawley rat brains. Brain blocks near midline were sliced into 8 μm sections and mounted on slides. Slides likely to contain RMS were deparaffinized, rehydrated, and underwent heat-induced epitope retrieval in TRIS-EDTA. Slides were then blocked in horse serum for 30 minutes. Primary antibodies were applied in 1× Optimax buffer overnight at 4° C. including mouse anti-Ezrin (1:50) (Sigma-Aldrich Cat #E8897, RRID: AB_476955), goat anti-glial fibrillary acidic protein (GFAP) (1:1000) (Abcam Cat #ab53554, RRID: AB_880202), and rabbit anti-Robo2 (1:50) (Novus Cat #NBP1-81399, RRID: AB_11013687). Slides were then rinsed in PBS/Tween and incubated in Alexa secondary antibodies (1:500) in 1× Optimax buffer for 1 hour at room temperature. Secondary antibodies included donkey anti-mouse 488 (1:500) (Thermo Fisher Scientific Cat #: A-21202, RRID: AB_141607), donkey anti-goat 568 (1:500) (Thermo Fisher Scientific Cat #: A-11057, RRID: AB_2534104), and donkey anti-rabbit 647 (1:500). Slides were then rinsed and Hoechst solution (1:10,000) (Invitrogen H3570) was applied for 5 minutes to label nuclei. Finally, slides were rinsed, coverslipped with fluoromount G, sealed with nail polish, and stored at 4° C.

Cultures and columns were fixed with 4% formaldehyde for 35 minutes at room temperature, rinsed with PBS, permeabilized with 0.3% Triton X-100 at room temperature for 20 minutes, blocked with 4% normal horse serum at room temperature for one hour, and again rinsed with PBS. Cultures were then incubated in primary antibody solutions at 4° C. for 16 hours. All cultures and columns were incubated in Hoechst solution (1:1000) (Invitrogen Cat #: H3570) during primary incubation. Subsequently, cultures were rinsed and incubated in appropriate Alexa secondary antibodies (1:500) in the dark at 37° C. for 2 hours. Rat cortical astrocyte planar cultures, extracted rat cortical astrocyte TE-RMSs, human gingiva stem cell-derived planar cultures, and extracted human gingiva stem cell-derived TE-RMSs were incubated in mouse anti-Ezrin (1:100) (Sigma-Aldrich Cat #E8897, RRID: AB_476955), goat anti-GFAP (1:1000) (Abcam Cat #ab53554, RRID: AB_880202), and rabbit anti-Robo2 (1:50) (Novus Cat #NBP1-81399, RRID: AB_11013687) followed by secondary antibodies donkey anti-mouse 488 (Thermo Fisher Scientific Cat #: A-21202, RRID: AB_141607), donkey anti-goat 568 (Thermo Fisher Scientific Cat #: A-11057, RRID: AB_2534104), and donkey anti-rabbit 647 (Thermo Fisher Scientific Cat #: A-31573, RRID: AB_2536183). To verify astrocytic phenotype, human gingiva stem cell-derived astrocyte-like planar cultures were incubated in mouse anti-CD31 (1:100) (Bio-Rad Cat #: MCA1746GA, RRID: AB_2832958) to label endothelial cells, and guinea pig anti-S100B (1:200) (Synaptic systems Cat #: 287 004, RRID: AB_2620025), chicken anti-GFAP (1:1000) (Abcam Cat #: ab4674, RRID: AB_304558), rabbit anti-GLAST (EAAT1) (Abcam Cat #: ab41751, RRID: AB_955879), and mouse anti-glutamine synthetase (Abcam Cat #: ab64613, RRID: AB_1140869) to label astrocytes; secondary antibodies were donkey anti-mouse 488 (Thermo Fisher Scientific Cat #: A-21202, RRID: AB_141607), donkey anti-guinea pig 568 (Sigma Cat #: SAB4600469, RRID: AB_2832959), donkey anti-rabbit 568 (Thermo Fisher Scientific Cat #: A10042, RRID: AB_2534017), and donkey anti-chicken 647 (Jackson Immunoresearch Cat #: 703-605-155, RRID: AB_2340379). Fixed human GMSC-derived TE-RMSs loaded with cortical neuronal aggregates were incubated in mouse anti-human nuclei (1:200) (Millipore Cat #: MAB1281, RRID: AB_94090) to label human astrocytes, rabbit anti-beta III tubulin (TuJ1) (1:500) (Abcam Cat #: ab18207, RRID: AB_444319) to label immature migrating neurons, and goat anti-GFAP (Abcam Cat #ab53554, RRID: AB_880202)) to label astrocytes in TE-RMSs; followed by secondary antibodies donkey anti-mouse 488 (Thermo Fisher Scientific Cat #: A-21202, RRID: AB_141607), donkey anti-rabbit 568 (Thermo Fisher Scientific Cat #: A10042, RRID: AB_2534017), and donkey anti-goat 647 (Thermo Fisher Scientific Cat #A-21447, RRID: AB_2535864). ECM-only columns were stained with rabbit anti-collagen (1:100) (Abcam Cat #: ab34710, RRID: AB_731684) or rabbit anti-laminin (1:500) (Abcam Cat #: ab11575, RRID: AB_298179) followed by secondary staining with donkey anti-mouse 488 (Thermo Fisher Scientific Cat #: A-21202, RRID: AB_141607) and donkey anti-rabbit 568 (Thermo Fisher Scientific Cat #: A10042, RRID: AB_2534017). All cultures and constructs were rinsed following secondary antibody staining. Cultures and constructs in columns were stored in PBS at 4° C. Coverslips containing extracted TE-RMSs were rinsed once in deionized water and mounted onto glass slides with fluoromount G. The edges of the slides were sealed with nail polish and stored at 4° C.

Six days after TE-RMS and control column implantation subjects were anesthetized with Euthasol and fixed via transcardial perfusion with 0.1% heparinized saline followed by 4% paraformaldehyde. Brains were extracted and submerged in formalin for 24 hours. Brains were sagittally blocked, embedded in paraffin, sliced into 8 μm sections, and mounted on slides. Slides were deparaffinized, rehydrated, and underwent heat-induced epitope retrieval in TRIS-EDTA. Slides were then blocked in horse serum for 30 minutes. Primary antibodies were applied in 1× Optimax buffer overnight at 4° C.: mouse anti-human nuclei (1:500) (Millipore Cat #: MAB1281, RRID: AB_94090) to label human astrocytes, goat anti-doublecortin (DCX) (1:500) (Novus Cat #: NBP1-72042, RRID: AB_11019667) to label immature migrating neurons, and either chicken anti-GFAP (1:1000) (Abcam Cat #: ab4674, RRID: AB_304558) or rabbit anti-collagen (1:100) (Abcam Cat #: ab34710, RRID: AB_731684). Slides were then rinsed in PBS/Tween and incubated in Alexa secondary antibodies (1:500) in 1× Optimax buffer for 1 hour at room temperature: donkey anti-mouse 488 (Thermo Fisher Scientific Cat #: A-21202, RRID: AB_141607), donkey anti-goat 568 (Thermo Fisher Scientific Cat #: A-11057, RRID: AB_2534104), donkey anti-chicken 647 (Jackson Immunoresearch Cat #: 703-605-155, RRID: AB_2340379), or donkey anti-rabbit 647 (Thermo Fisher Scientific Cat #: A-31573, RRID: AB_2536183). Slides were rinsed and Hoechst solution (1:10,000) (Invitrogen H3570) was applied for 5 minutes. Slides were rinsed, coverslipped with fluoromount G, sealed with nail polish, and stored at 4° C.

### Western Blot Analysis

Planar-cultured GMSCs or astrocytes induced from GMSCs were harvested and whole cell lysates were prepared by incubation with radioimmunoprecipitation assay (RIPA) buffer (Santa Cruz) supplemented with a cocktail of protease inhibitors (Santa Cruz) and the total protein concentrations were determined using bicinchoninic acid (BCA) method (BioVision). Then 30 μg of proteins per well were subjected to SDS-polyacrylamide gel electrophoresis before being electroblotted onto a 0.2 μm nitrocellulose membrane (GE Healthcare). After blocking with 5% nonfat dry milk in TBST (25 mmol/L Tris, pH, 7.4, 137 mmol/L NaCl, 0.5% Tween20), membranes were incubated overnight at 4° C. with following primary antibodies: GFAP (1:1000, ab53554, Abcam), glutamine synthetase (1:1000, ab64613, Abcam), or GAPDH (1:2000, #5174, Cell Signaling) as loading control. After extensively washing, membranes were incubated with horseradish peroxidase (HRP)-conjugated secondary antibodies (Santa Cruz) and blot signals were developed with ECL™ Western Blotting Detect Reagents (GE Health Care) and scanned using Amersham Imager 680.

### Imaging

Cultures and constructs were routinely imaged for observation using phase contrast and epifluorescence microscopy on a Nikon Inverted Eclipse Ti-S microscope with digital image acquisition using a QiClick camera interfaced with Nikon Elements Basic Research software (4.10.01). Epifluorescence images for analysis were captured using a Nikon Eclipse Ti-S inverted epi-fluorescent scope outfitted with an Andor Zyla sCMOS 5.5 megapixel camera interfaced with Nikon Elements Basic Research software (4.10.01) with either a 10× (Plan Apo Lambda 10×, n.a. 0.45) or 20× (Plan Apo Lambda 10×, n.a. 0.75) objective. All images acquired for comparative analyses were captured with identical acquisition settings. Samples were also fluorescently imaged using a Nikon A1Rsi Laser Scanning Confocal microscope with a 10×, 20×, or 60× objective (CFI Plan Apo Lambda 10×, n.a. 0.45; 20×, n.a. 0.75; or 60× Oil, n.a. 1.40).

### Imaging Analyses, Statistics, and Reproducibility

Image processing and analyses were performed using the freely available FIJI (Fiji Is Just ImageJ) software platform⁸⁵. Values reported in the Results section are mean±SEM, unless otherwise noted. Statistical testing was performed in GraphPad Prism 8 for Windows 64 bit. Due to the obvious differences between protoplasmic and TE-RMS astrocytes, blinding was not possible. Therefore, we minimized potential bias by maximizing automation via design and application of macros for automated image processing and analyses. All Nikon nd2 files were imported into FIJI via the Bioformats function and each channel was split into an individual grayscale Tiff. Background subtraction was applied to all images using the rolling ball method with a diameter of 100 pixels.

To compare endogenous rat RMS glial tube and protoplasmic astrocytes as summarized in FIG. 2, standardized 75 μm×75 μm square region of interest (ROI) was utilized to isolate an RMS field and a protoplasmic astrocyte field for each brain analyzed (n=5). To isolate reliably astrocytic signal in each channel, binary masks were first created from the GFAP channel using the Max Entropy thresholding method followed by the Analyze Particles function to remove noisy particles smaller than 0.1 μm². For each ROI, the Image Calculator “AND” function were used to create a new image containing signal only where there was signal in both the binary GFAP mask “AND” the raw image from another channel. This effectively uses the GFAP binary mask to cut out astrocyte-shaped areas from each channel of the ROI for analysis of astrocytic signal for each protein. The mean intensity for the astrocytic signal in each channel of each ROI was then measured. Since each brain produced an RMS and Protoplasmic ROI pair, mean intensities of RMS and protoplasmic astrocytes were compared by two-tailed paired Student's t-tests for each channel.

To compare TE-RMS astrocytes and astrocytes from planar sister cultures as summarized in FIG. 3 (rat; TE-RMS n=9, sister n=6) and FIG. 5 (human; TE-RMS n=5, sister n=6), masking to isolate astrocytic signal was not necessary since the experiments utilized astrocytes in culture. Instead, mean intensities were measured for the entire field of view (standardized due to identical acquisition settings for all comparisons) for each channel of each image. Those mean intensities were then normalized to the amount of cells in each image. The Hoechst channel for each image was converted to a binary mask using the MaxEntropy thresholding method followed by the analyze particles function to remove noisy particles smaller than 0.1 μm², and the total nuclear area was then measured and used for normalization as the total “amount of cells” in each field of view. Mean intensities of TE-RMS and planar culture astrocytes were compared by two-tailed Student's t-tests for each channel.

To compare migrating neurons in our in vitro migration assay, the Hoechst channel for each image was first converted to a binary mask in FIJI. For the TE-RMS group, the human nuclei channel was also converted to a binary mask and then subtracted from the corresponding Hoechst channel using the Image Calculator “Subtract” function in FIJI to remove TE-RMS nuclei and isolate signal from migrating rat neurons. Partially removed human nuclei still present after the subtraction function (evident as open circles) were removed via the Binary>Open function in FIJI. Particle counts were unreliable due to inconsistencies resolving adjacent and overlapping nuclei, therefore the total nuclear area was measured via the “Analyze Particles” function in FIJI. For each assay, measurements were taken from two ROIs spanning the width of the inner lumen of the microcolumns (300 μm). The first ROI extended 1 mm from the edge of the neuronal aggregate, and the second ROI extended an additional 2.5 mm from the end of the first (extending from 1 to 3.5 mm from the edge of the aggregate). Mean nuclear areas were compared via one-way ANOVA with Bonferroni adjustment for multiple comparisons. A Log transform for all values was performed to meet assumptions of normality and equal variance. Neurite extension from neuronal aggregates in collagen and collagen/laminin columns was measured from the edge of the aggregate to the end of the longest Tuj1-positive neurite using the line segment measuring tool in FIJI. The analyst was blinded to ECM composition. Statistical comparisons were not performed after removing the blind because collagen-only columns did not exhibit any measurable neurite outgrowth.

### Example 1 Cited Documents

1. Zhao, C., Deng, W. & Gage, F. H. Mechanisms and functional implications of adult neurogenesis. Cell 132, 645-60 (2008).
2. Ming, G.- L. & Song, H. Adult neurogenesis in the mammalian brain: significant answers and significant questions. Neuron 70, 687-702 (2011).
3. Lim, D. A. & Alvarez-Buylla, A. The Adult Ventricular-Subventricular Zone (V-SVZ) and Olfactory Bulb (OB) Neurogenesis. Cold Spring Harbor perspectives in biology 8, (2016).
4. Lledo, P.- M., Merkle, F. T. & Alvarez-Buylla, A. Origin and function of olfactory bulb interneuron diversity. Trends in neurosciences 31, 392-400 (2008).
5. Brill, M. S. et al. Adult generation of glutamatergic olfactory bulb interneurons. Nature neuroscience 12, 1524-33 (2009).
6. Lazarini, F. & Lledo, P.- M. Is adult neurogenesis essential for olfaction? Trends in neurosciences 34, 20-30 (2011).
7. Nam, S. C. et al. Dynamic features of postnatal subventricular zone cell motility: A two-photon time-lapse study. The Journal of Comparative Neurology 505, 190-208 (2007).
8. Kojima, T. et al. Subventricular Zone-Derived Neural Progenitor Cells Migrate Along a Blood Vessel Scaffold Toward the Post-Stroke Striatum. STEM CELLS N/A-N/A (2010) doi:10.1002/stem.306.
9. Grade, S. et al. Brain-derived neurotrophic factor promotes vasculature-associated migration of neuronal precursors toward the ischemic striatum. PloS one 8, e55039 (2013).
10. Ota, H. et al. Speed control for neuronal migration in the postnatal brain by Gmip-mediated local inactivation of RhoA. Nature Communications 5, 4532 (2014).
11. Kaneko, N., Sawada, M. & Sawamoto, K. Mechanisms of neuronal migration in the adult brain. Journal of neurochemistry 141, 835-847 (2017).
12. Dillen, Y., Kemps, H., Gervois, P., Wolfs, E. & Bronckaers, A. Adult Neurogenesis in the Subventricular Zone and Its Regulation After Ischemic Stroke: Implications for Therapeutic Approaches. Translational stroke research (2019) doi: 10.1007/s12975-019-00717-8.
13. Fujioka, T., Kaneko, N. & Sawamoto, K. Blood vessels as a scaffold for neuronal migration. Neurochemistry international 126, 69-73 (2019).
14. Kaneko, N. et al. New neurons use Slit-Robo signaling to migrate through the glial meshwork and approach a lesion for functional regeneration. Science advances 4, eaav0618 (2018).
15. Cleary, M. A., Uboha, N., Picciotto, M. R. & Beech, R. D. Expression of ezrin in glial tubes in the adult subventricular zone and rostral migratory stream. Neuroscience 143, 851-61 (2006).
16. Tang, H. et al. Effect of neural precursor proliferation level on neurogenesis in rat brain during aging and after focal ischemia. Neurobiology of Aging 30, 299-308 (2009).
17. Lu, J., Manaenko, A. & Hu, Q. Targeting Adult Neurogenesis for Poststroke Therapy. Stem cells international 2017, U.S. Pat. No. 5,868,632 (2017).
18. Chang, E. H. et al. Traumatic Brain Injury Activation of the Adult Subventricular Zone Neurogenic Niche. Frontiers in neuroscience 10, 332 (2016).
19. Arvidsson, A., Collin, T., Kirik, D., Kokaia, Z. & Lindvall, O. Neuronal replacement from endogenous precursors in the adult brain after stroke. Nature medicine 8, 963-70 (2002).
20. Parent, J. M., Vexler, Z. S., Gong, C., Derugin, N. & Ferriero, D. M. Rat forebrain neurogenesis and striatal neuron replacement after focal stroke. Annals of neurology 52, 802-13 (2002).
21. Jin, K. et al. Directed migration of neuronal precursors into the ischemic cerebral cortex and striatum. Molecular and Cellular Neuroscience 24, 171-189 (2003).
22. Thored, P. et al. Persistent Production of Neurons from Adult Brain Stem Cells During Recovery after Stroke. Stem Cells 24, 739-747 (2006).
23. Thored, P. et al. Long-term neuroblast migration along blood vessels in an area with transient angiogenesis and increased vascularization after stroke. Stroke 38, 3032-9 (2007).
24 Yamashita, T. et al. Subventricular zone-derived neuroblasts migrate and differentiate into mature neurons in the post-stroke adult striatum. The Journal of neuroscience: the official journal of the Society for Neuroscience 26, 6627-36 (2006).
25. Liu, X. S. et al. Gene profiles and electrophysiology of doublecortin-expressing cells in the subventricular zone after ischemic stroke. Journal of cerebral blood flow and metabolism: official journal of the International Society of Cerebral Blood Flow and Metabolism 29, 297-307 (2009).
26 Kernie, S. G. & Parent, J. M. Forebrain neurogenesis after focal Ischemic and traumatic brain injury. Neurobiology of disease 37, 267-74 (2010).
27. Young, C. C., Brooks, K. J., Buchan, A. M. & Szele, F. G. Cellular and molecular determinants of stroke-induced changes in subventricular zone cell migration. Antioxidants & redox signaling 14, 1877-88 (2011).
28. Lindvall, O. & Kokaia, Z. Neurogenesis following Stroke Affecting the Adult Brain. Cold Spring Harbor perspectives in biology 7, (2015).
29. Ramaswamy, S., Goings, G. E., Soderstrom, K. E., Szele, F. G. & Kozlowski, D. A. Cellular proliferation and migration following a controlled cortical impact in the mouse. Brain Research 1053, 38-53 (2005).
30. Acosta, S. A. et al. Long-term upregulation of inflammation and suppression of cell proliferation in the brain of adult rats exposed to traumatic brain injury using the controlled cortical impact model. PloS one 8, e53376 (2013).
31. Mierzwa, A. J., Sullivan, G. M., Beer, L. A., Ahn, S. & Armstrong, R. C. Comparison of cortical and white matter traumatic brain injury models reveals differential effects in the subventricular zone and divergent Sonic hedgehog signaling pathways in neuroblasts and oligodendrocyte progenitors. ASN neuro 6, (2014).
32. Chirumamilla, S., Sun, D., Bullock, M. R. & Colello, R. J. Traumatic brain injury induced cell proliferation in the adult mammalian central nervous system. Journal of neurotrauma 19, 693-703 (2002).
33. Chen, X.- H., Iwata, A., Nonaka, M., Browne, K. D. & Smith, D. H. Neurogenesis and Glial Proliferation Persist for at Least One Year in the Subventricular Zone Following Brain Trauma in Rats. Journal of Neurotrauma 20, 623-631 (2003).
34. Zhang, R. L. et al. Reduction of the Cell Cycle Length by Decreasing G 1 Phase and Cell Cycle Reentry Expand Neuronal Progenitor Cells in the Subventricular Zone of Adult Rat after Stroke. Journal of Cerebral Blood Flow & Metabolism 26, 857-863 (2006).
35. Addington, C. P., Roussas, A., Dutta, D. & Stabenfeldt, S. E. Endogenous Repair Signaling after Brain Injury and Complementary Bioengineering Approaches to Enhance Neural Regeneration. Biomarker Insights 10s1, BMI.S20062 (2015).
36 Hayashi, Y., Jinnou, H., Sawamoto, K. & Hitoshi, S. Adult neurogenesis and its role in brain injury and psychiatric diseases. Journal of neurochemistry 147, 584-594 (2018).
37. Ohab, J. J., Fleming, S., Blesch, A. & Carmichael, S. T. A Neurovascular Niche for Neurogenesis after Stroke. Journal of Neuroscience 26, 13007-13016 (2006).
38 Wang, Z. et al. Neurogenic Niche Conversion Strategy Induces Migration and Functional Neuronal Differentiation of Neural Precursor Cells Following Brain Injury. Stem Cells and Development scd.2019.0147 (2020) doi: 10.1089/scd.2019.0147.
39. Kolb, B. et al. Growth factor-stimulated generation of new cortical tissue and functional recovery after stroke damage to the motor cortex of rats. Journal of cerebral blood flow and metabolism: official journal of the International Society of Cerebral Blood Flow and Metabolism 27, 983-97 (2007).
40. Schäbitz, W.- R. et al. Intravenous Brain-Derived Neurotrophic Factor Enhances Poststroke Sensorimotor Recovery and Stimulates Neurogenesis. Stroke 38, 2165-2172 (2007).
41. Ma, M. et al. Intranasal delivery of transforming growth factor-betal in mice after stroke reduces infarct volume and increases neurogenesis in the subventricular zone. BMC neuroscience 9, 117 (2008).
42. Petraglia, A. L., Marky, A. H., Walker, C., Thiyagarajan, M. & Zlokovic, B. V. Activated protein C is neuroprotective and mediates new blood vessel formation and neurogenesis after controlled cortical impact. Neurosurgery 66, 165-71; discussion 171-2 (2010).
43. Popa-Wagner, A. et al. Effects of Granulocyte-Colony Stimulating Factor After Stroke in Aged Rats. Stroke 41, 1027-1031 (2010).
44. Erlandsson, A., Lin, C.- H. A., Yu, F. & Morshead, C. M. Immunosuppression promotes endogenous neural stem and progenitor cell migration and tissue regeneration after ischemic injury. Experimental Neurology 230, 48-57 (2011).
45. Yu, S.- J. et al. Local administration of AAV-BDNF to subventricular zone induces functional recovery in stroke rats. PloS one 8, e81750 (2013).
46. Clark, A. R., Carter, A. B., Hager, L. E. & Price, E. M. In Vivo Neural Tissue Engineering: Cylindrical Biocompatible Hydrogels That Create New Neural Tracts in the Adult Mammalian Brain. Stem Cells and Development 25, 1109-1118 (2016).
47. Gundelach, J. & Koch, M. Redirection of neuroblast migration from the rostral migratory stream into a lesion in the prefrontal cortex of adult rats. Experimental brain research 236, 1181-1191 (2018).
48. Jinnou, H. et al. Radial Glial Fibers Promote Neuronal Migration and Functional Recovery after Neonatal Brain Injury. Cell stem cell 22, 128-137.e9 (2018).
49. Purvis, E. M., O'Donnell, J. C., Chen, H. I. & Cullen, D. K. Tissue engineering and biomaterial strategies to elicit endogenous neuronal replacement in the brain. Frontiers in neurology (2020) doi: 10.3389/fneur.2020.00344.
50. Cullen, D. K. et al. Microtissue engineered constructs with living axons for targeted nervous system reconstruction. Tissue engineering. Part A 18, 2280-9 (2012).
51. Struzyna, L. A., Katiyar, K. & Cullen, D. K. Living scaffolds for neuroregeneration. Current Opinion in Solid State and Materials Science 18, 308-318 (2014).
52. Struzyna, L. A. et al. Rebuilding Brain Circuitry with Living Micro-Tissue Engineered Neural Networks. Tissue engineering. Part A 21, 2744-56 (2015).
53 Struzyna, L. A. et al. Tissue engineered nigrostriatal pathway for treatment of Parkinson's disease. Journal of tissue engineering and regenerative medicine 12, 1702-1716 (2018).
54. Adewole, D. O., Das, S., Petrov, D. & Cullen, D. K. Scaffolds for brain tissue reconstruction. in Handbook of Tissue Engineering Scaffolds: Volume Two 3-29 (Elsevier, 2019). doi:10.1016/B978-O-08-102561-1.00001-4.
55. Katiyar, K. S., Struzyna, L. A., Das, S. & Cullen, D. K. Stretch growth of motor axons in custom mechanobioreactors to generate long-projecting axonal constructs. Journal of Tissue Engineering and Regenerative Medicine 13, 2040-2054 (2019).
56. Winter, C. C. et al. Transplantable living scaffolds comprised of micro-tissue engineered aligned astrocyte networks to facilitate central nervous system regeneration. Acta biomaterialia 38, 44-58 (2016).
57. Katiyar, K. S. et al. Three-dimensional Tissue Engineered Aligned Astrocyte Networks to Recapitulate Developmental Mechanisms and Facilitate Nervous System Regeneration. Journal of visualized experiments: JoVE (2018) doi: 10.3791/55848.
58. O'Donnell, J., Katiyar, K., Panzer, K. & Cullen, Dk. A tissue-engineered rostral migratory stream for directed neuronal replacement. Neural Regeneration Research 13, 1327 (2018).
59 Ganz, J. et al. Astrocyte-like cells derived from human oral mucosa stem cells provide neuroprotection in vitro and in vivo. Stem cells translational medicine 3, 375-86 (2014).
60. Panzer, K. V. et al. Tissue Engineered Bands of Büngner for Accelerated Motor and Sensory Axonal Outgrowth. Front. Bioeng. Biotechnol. 8, (2020).
61. Lois, C. & Alvarez-Buylla, A. Long-distance neuronal migration in the adult mammalian brain. Science 264, 1145-1148 (1994).
62. Yin, X. et al. Neurons Derived from Human Induced Pluripotent Stem Cells Integrate into Rat Brain Circuits and Maintain Both Excitatory and Inhibitory Synaptic Activities. eneuro 6, ENEURO.0148-19.2019 (2019).
63. Rolfe, Andrew; Sun, D. Stem Cell Therapy in Brain Trauma: Implications for Repair and Regeneration of Injured Brain in Experimental TBI Models. in Brain Neurotrauma: Molecular, Neuropsychological, and Rehabilitation Aspects. (ed. Kobeissy, F. H.) (CRC Press/Taylor & Francis, 2015).
64. Yamashita, T. et al. Novel Therapeutic Transplantation of Induced Neural Stem Cells for Stroke. Cell transplantation 26, 461-467 (2017).
65. Xiong, L.- L. et al. Neural Stem Cell Transplantation Promotes Functional Recovery from Traumatic Brain Injury via Brain Derived Neurotrophic Factor-Mediated Neuroplasticity. Molecular Neurobiology 55, 2696-2711 (2018).
66. Kim, K. et al. Epigenetic memory in induced pluripotent stem cells. Nature 467, 285-90 (2010).
67. Hickey, K. & Stabenfeldt, S. E. Using biomaterials to modulate chemotactic signaling for central nervous system repair. Biomedical Materials 13, 044106 (2018).
68. Cotman, C; Berchtold, N. Exercise: a behavioral intervention to enhance brain health and plasticity. Trends in Neurosciences 25, 295-301 (2002).
69. Griesbach, G. S., Hovda, D. A., Molteni, R., Wu, A. & Gomez-Pinilla, F. Voluntary exercise following traumatic brain injury: brain-derived neurotrophic factor upregulation and recovery of function. Neuroscience 125, 129-139 (2004).
70 Fon, D. et al. Nanofibrous scaffolds releasing a small molecule BDNF-mimetic for the re-direction of endogenous neuroblast migration in the brain. Biomaterials 35, 2692-712 (2014).
71. Motalleb, R. et al. In vivo migration of endogenous brain progenitor cells guided by an injectable peptide amphiphile biomaterial. Journal of tissue engineering and regenerative medicine 12, e2123-e2133 (2018).
72. Ajioka, I. et al. Enhancement of neuroblast migration into the injured cerebral cortex using laminin-containing porous sponge. Tissue engineering. Part A 21, 193-201 (2015).
73. Fujioka, T. et al. B1 integrin signaling promotes neuronal migration along vascular scaffolds in the post-stroke brain. EBioMedicine 16, 195-203 (2017).
74. Gengatharan, A., Bammann, R. R. & Saghatelyan, A. The Role of Astrocytes in the Generation, Migration, and Integration of New Neurons in the Adult Olfactory Bulb. Frontiers in neuroscience 10, 149 (2016).
75. Mason, H. A., Ito, S. & Corfas, G. Extracellular signals that regulate the tangential migration of olfactory bulb neuronal precursors: inducers, inhibitors, and repellents. The Journal of neuroscience: the official journal of the Society for Neuroscience 21, 7654-63 (2001).
76. Garcia-Marqués, J., De Carlos, J. A., Greer, C. A. & López-Mascaraque, L. Different astroglia permissivity controls the migration of olfactory bulb interneuron precursors. Glia 58, 218-230 (2010).
77 Persson, A., Lindwall, C., Curtis, M. A. & Kuhn, H. G. Expression of ezrin radixin moesin proteins in the adult subventricular zone and the rostral migratory stream. Neuroscience 167, 312-22 (2010).
78. Kaneko, N. et al. New neurons clear the path of astrocytic processes for their rapid migration in the adult brain. Neuron 67, 213-23 (2010).
79 Harris, J. P. et al. Advanced biomaterial strategies to transplant preformed micro-tissue engineered neural networks into the brain. Journal of neural engineering 13, 016019 (2016).
80. Serruya, M. D. et al. Engineered Axonal Tracts as “Living Electrodes” for Synaptic-Based Modulation of Neural Circuitry. Advanced Functional Materials 28, 1701183 (2018).
81. Li, L. et al. Human Embryonic Stem Cells Possess Immune-Privileged Properties. Stem Cells 22, 448-456 (2004).
82. Zhang, Q. et al. Mesenchymal stem cells derived from human gingiva are capable of immunomodulatory functions and ameliorate inflammation-related tissue destruction in experimental colitis. Journal of immunology (Baltimore, Md.: 1950) 183, 7787-98 (2009).
83. Zhang, Q. et al. Neural Crest Stem-Like Cells Non-genetically Induced from Human Gingiva-Derived Mesenchymal Stem Cells Promote Facial Nerve Regeneration in Rats. Molecular Neurobiology 55, 6965-6983 (2018).
84. Zhang, Q. et al. 3D bio-printed scaffold-free nerve constructs with human gingiva-derived mesenchymal stem cells promote rat facial nerve regeneration. Scientific Reports 8, 6634 (2018).
85. Schindelin, J. et al. Fiji: an open-source platform for biological-image analysis. Nature Methods 9, 676-682 (2012).

### Example 2: Unique Astrocyte Cytoskeletal and Nuclear Morphology in a Three-Dimensional Tissue-Engineered Rostral Migratory Stream

### Materials and Methods

### Astrocyte Cell Culture

Primary cortical astrocytes were harvested from Sprague-Dawley rat pups at postnatal day 0-1. Following dissociation (described previously [27]), astrocytes were cultured in DMEM/F12 medium supplemented with 10% Fetal Bovine Serum and 1% Penicillin-Streptomycin antibiotics in a cell culture incubator maintained at 37° C. and 5% CO₂. Astrocytes were passaged at 80% confluency. Astrocytes between passages 4-10 were utilized for fabrication of TE-RMSs and planar astrocyte cultures.

### Fabrication of Hydrogel Micro-Columns

TE-RMSs were fabricated in hydrogel micro-columns made of 3% agarose. Fabrication of micro-columns has been described previously [27,30,31]. Briefly, warm agarose dissolved in Dulbecoo's phosphate-buffered saline (DPBS) was drawn into a capillary tube (inner diameter=701 microns) with a needle (diameter=300 microns) in the center. Following agarose cooling and capillary tube removal, the micro-column was pushed off the needle into PBS, effectively creating micro-columns with an inner diameter of 300 microns and an outer diameter of 701 microns. Previous experiments have determined these dimensions to be optimal for astrocyte bundling and TE-RMS formation [26].

### Fabrication of Tissue-Engineered Rostral Migratory Streams and Planar Astrocyte Cultures

Fabrication of TE-RMSs has been recently described [29]. Micro-columns were cut to a 4 mm length and the inner lumen was seeded with 1 mg/mL rat tail type 1 collagen (Corning Catalog #354236) diluted in in serum free co-culture media consisting of Neurobasal medium supplemented with 2% B27, 1% G-5, 0.25% L-glutamine, and 1% penicillin-streptomycinastrocyte culture media. Columns were placed in an incubator at 37° C. and 5% CO₂ for around 3 h until collagen completely polymerized to coat the inner walls of the micro-columns. During the collagen polymerization period, a flask of 80% confluent astrocytes was passaged and cells were resuspended in serum free co-culture media consisting of Neurobasal medium supplemented with 2% B27, 1% G-5, 0.25% L-glutamine, and 1% penicillin-streptomycin. Following complete collagen polymerization, micro-columns were seeded with astrocytes in co-culture media at a density of around 1 mil-lion cells/mL. Columns were then returned to the incubator at 37° C. and 5% CO₂ and one hour later the outer edges of the columns were reinforced with 1 mg/mL collagen. Columns were incubated for another hour at 37° C. and 5% CO₂, dishes were flooded with warm co-culture media, and then columns were returned to the incubator at 37° C. and 5% CO₂. Under these conditions, astrocytes bundle together with collagen and self-assemble into cords of longitudinally aligned astrocytes with bidirectional processes to form TE-RMSs. All experiments herein investigated TE-RMSs at 24 h following astrocyte seeding into microcolumns. For planar astrocyte samples, a flask of 80% confluent astrocytes was passaged, cells were resuspended in co-culture media, and were plated on top of polymerized 1 mg/mL collagen in a 12-well plate at a density of around 1 million cells/mL in co-culture media. For 100× fluorescent imaging of planar astrocytes, cells were cultured in 12-well plates on top of poly-L-lysine (PLL) and collagen-coated glass coverslips. For scanning electron microscopy (SEM) imaging, planar astrocytes were cultured in 24-well plates on top of PLL-coated glass coverslips.

### Immunocytochemistry

Following TE-RMS formation, constructs were extracted from micro-columns onto poly-1-lysine glass slides for immunocytochemical analyses as previously described [29]. Planar astrocyte cultures and extracted TE-RMSs were fixed with 4% paraformaldehyde for 30 min at room temperature. Following rinsing with PBS, cultures and constructs were permeabilized with 0.3% Triton X-100 and blocked with 4% normal horse serum for one hour at room temperature and again rinsed with PBS. Cultures and constructs were incubated in the primary antibody goat anti-glial fibrillary acidic protein (GFAP) (1:1000) (Abcam Cat #ab53554, RRID: AB_880202) overnight at 4° C. Cultures were then rinsed and incubated in the Alexa secondary antibody donkey anti-goat 568 (1:500) (Thermo Fisher Scientific Catalog #: A-11057, RRID: AB_2534104) and Hoechst solution (1:1000) (Invitrogen H3570) in the dark for two hours at 37° C.

### Immunohistochemistry

Five adult Sprague-Dawley rat brains from an archival tissue bank were selected for analysis of the endogenous RMS. Brains were formalin-fixed paraffin-embedded (FFPE) and sagittally sliced into 8-micron thick sections and mounted onto slides. Slides were deparaffinized, rehydrated, and placed in Tris-EDTA for heat-induced epitope retrieval. Slides were blocked in normal horse serum for 30 min and the primary antibody goat anti-GFAP (1:1000) (Abcam Cat #ab53554, RRID: AB_880202) was diluted in 1× Optimax buffer and applied overnight at 4° C. Slides were washed in PBS Tween and incubated in the dark with the Alexa secondary antibody donkey anti-goat 568 (1:500) (Thermo Fisher Scientific Catalog #: A-11057, RRID: AB_2534104). Slides were then rinsed and incubated in Hoechst solution (1:10,000) (Invitrogen H3570) for 5 min and then rinsed again. Cover slips were mounted with fluoromount G and stored at 4° C.

### Imaging

Fluorescent images of cultures, constructs, and brain slices were obtained using a Nikon A1Rsi Laser Scanning Confocal microscope with a ×10, ×20, ×60, or ×100 objective (CFI Plan Apo Lambda×10, n.a. 0.45; ×20, n.a. 0.75; ×60 Oil, n.a. 1.40; ×100, n.a. 1.45). Scanning electron microscope experiments were carried out at the CDB Microscopy Core (Perelman School of Medicine, University of Pennsylvania). Samples were washed three times with 50 mM Na-cacodylate buffer, fixed for 2 h with 2% glutaraldehyde in 50 mM Na-cacodylate buffer (pH 7.3), and dehydrated in a graded series of ethanol concentrations through 100% over a period of 2.5 h. Dehydration in 100% ethanol was done three times. Dehydrated samples were incubated for 20 min in 50% HMDS in ethanol followed by three changes of 100% HMDS (Sigma-Aldrich Co.) and followed by overnight air-drying as described previously [32]. Then samples were mounted on studs and sputter coated with gold palladium. Specimens were observed and photographed using a Quanta 250 FEG scanning electron microscope (FEI, Hillsboro, OR, USA) at 10 kV accelerating voltage.

### Imaging Analyses, Statistics, and Reproducibility

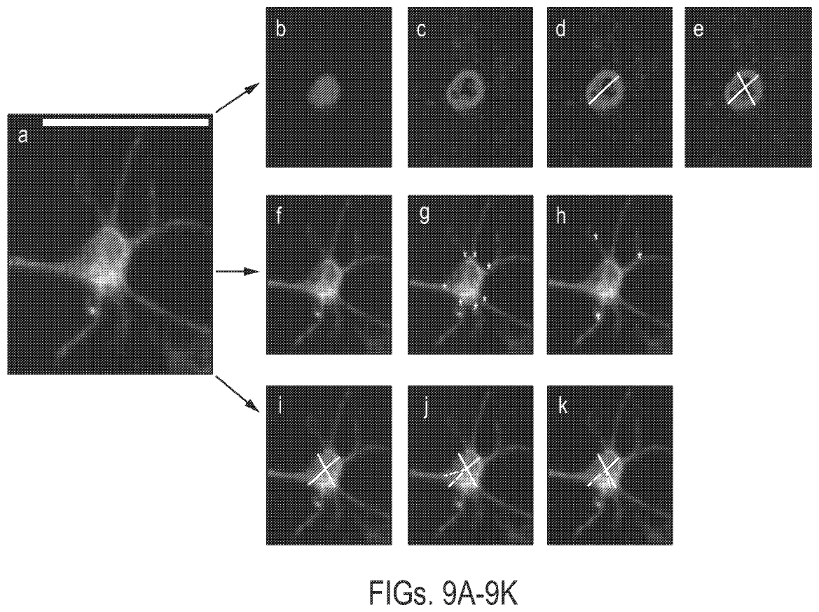

Image analyses were performed using FIJI (Fiji Is Just ImageJ) software [33]. Statistical testing was performed in GraphPad Prism 9 for macOS. 20× fluorescent confocal images were utilized for quantification of all nuclear and cytoskeletal measurements. Fluorescent images were imported into FIJI as Nikon nd2 files, split into two channels (Hoechst and GFAP), z-stacked and compressed, and underwent background subtraction (rolling ball method; 50 pixel diameter). In in vitro planar astrocyte and TE-RMS images, all cells with non-overlapping nuclei were quantified. Across TE-RMS samples, we estimate that about 50% of TE-RMS astrocytes were excluded from analysis due to the appearance of overlap-ping nuclei. In in vivo images, protoplasmic and RMS astrocytes were quantified only if they possessed visible, non-overlapping nuclei and clear GFAP process extensions. Endogenous RMS astrocytes were identified within the dense, distinct meshwork of GFAP processes extending from the lateral ventricle to the olfactory bulb, as reliably identified previously by our laboratory and others [29,34]. Protoplasmic astrocytes were identified as astrocytes outside of this distinct structure. To measure the long and short nuclear axes (FIGS. 9A-9E), the “find edges” FIJI function was applied to the Hoechst fluorescent channel. A cell's long nuclear axis was measured as the longest distance from the one edge of the nucleus to the other. A cell's short nuclear axis was measured from one edge of the nucleus to the other and perpendicular to the cell's long nuclear axis. The nuclear aspect ratio of each cell was found by dividing the long nuclear axis by the short nuclear axis. In the merged fluorescent image (GFAP+Hoechst channels), the number of main processes, number of branch points, and angle of each main process were quantified for each cell. Main processes (FIG. 9G) were defined as extensions of GFAP that arose directly from the cell's nucleus. Branch points (FIG. 2H) were defined as extensions of GFAP that arose from other GFAP extensions. The angle of each main process (FIGS. 9I-9K) was measured as it deviated from the long nuclear axis. To perform angle measurements, an angle was drawn with the first point of the angle placed on the cell process (at the point where the process contacted the cell body), the second point (middle) of the angle was placed on the intersection of the cell's long and short nuclear axes, and the third point of the angle placed on the end of the long nuclear axis (the end closer to the cell process). All angle measurements fell between 0 and 90 degrees, as 90 degrees was the maximum angle that could exist between the process and the long nuclear axis. All measurements for each cell were recorded and saved as ROIs in FIJI. For planar astrocyte cultures, 225 cells across 6 samples were quantified. For TE-RMS cultures, 275 cells across 9 samples were quantified. For in vivo samples (n=5 animals), 124 protoplasmic astrocytes and 131 RMS astrocytes were quantified. Nuclear aspect ratios, number of main processes, and number of branch points (planar versus TE-RMS; protoplasmic versus RMS) were compared by two-tailed nested t-tests, with values for individual cells nested within each culture. For angle measurements, the median angle of each cell was selected and these values (planar versus TE-RMS; protoplasmic versus RMS) were then compared by two-tailed nested t-tests.

### Results

### Planar Astrocytes Have a More Complex and Varied Cytoskeletal Arrangement Compared to TE-RMS Astrocytes

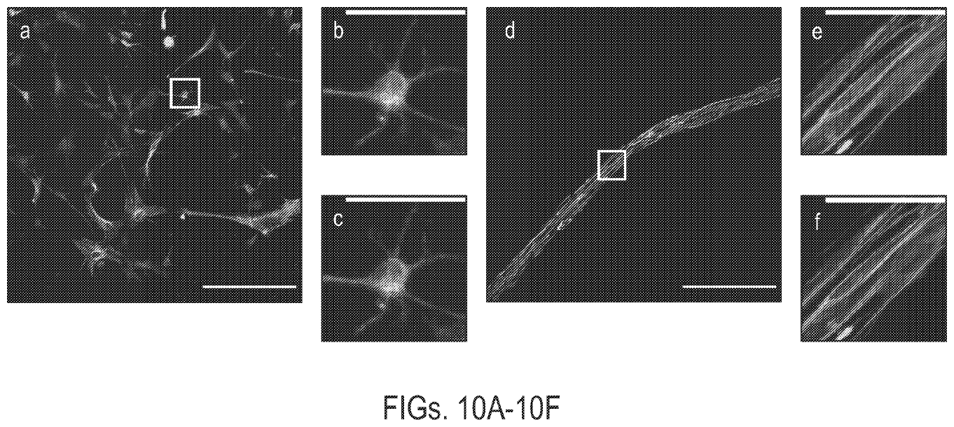

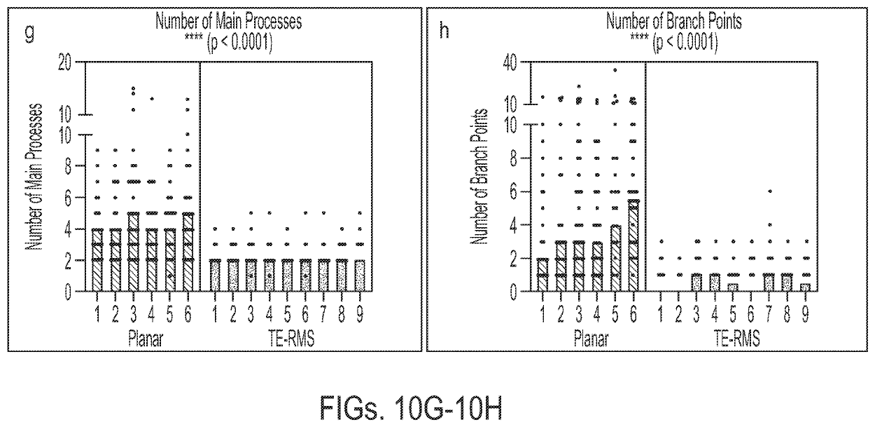

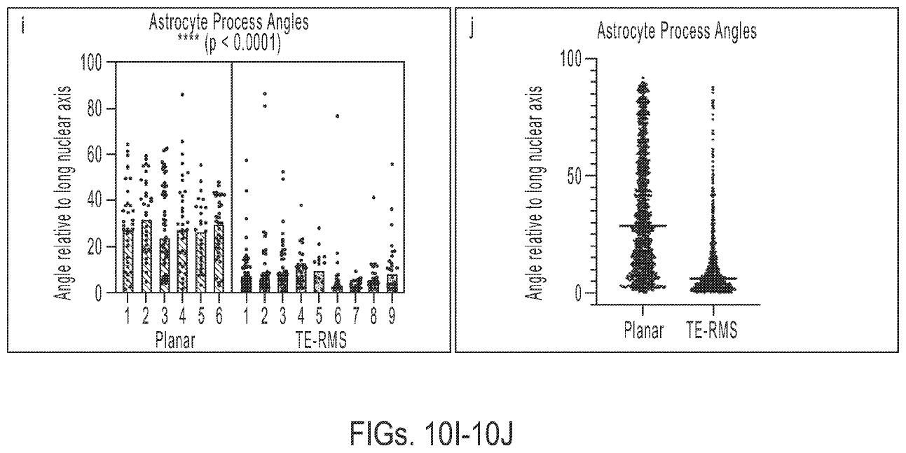

We used fluorescence immunocytochemistry (ICC) to label nuclei (Hoechst) and the intermediate filament protein glial fibrillary acidic protein (GFAP) in planar astrocyte (n=6) and TE-RMS (n=9) samples to analyze the overall complexity of the cytoskeletal arrangement of in vitro cultures. For each sample, a single 20× fluorescent confocal image was used for quantification. We quantified the number of main processes, number of branch points, and angle of each main process across planar astrocytes (n=225 cells) and TE-RMS astrocytes (n=275 cells). Example representative images of planar (FIG. 10A) and TE-RMS (FIG. 10D) samples highlight the distinct cytoskeletal arrangements observed between these two in vitro groups. All cells with non-overlapping nuclei were quantified. Comparing the number of main cell processes (defined as GFAP extensions protruding directly from the cell body) by two-tailed, nested t-test revealed that planar astrocytes had a significantly higher number of main processes compared to TE-RMS astrocytes (FIG. 10G; t=10.36, df=13, p<0.0001). Number of branch points (defined as GFAP extensions protruding from another GFAP extension) were also compared by two-tailed, nested t-test and revealed that planar astrocytes had a significantly higher number of branch points compared to TE-RMS astrocytes (FIG. 10H; t=8.801, df=14; p<0.0001). Planar astrocytes demonstrated high variability across main process and branch point measurements, highlighting the heterogeneity in planar astrocyte morphology. This variability is visibly reduced in TE-RMS astrocytes which exhibit a more uniform morphology. The angle of each main process was then measured relative to the long nuclear axis of the cell. Almost all analyzed cells had more than one main process and therefore more than one angular measurement. Since these measurements did not adhere to a Gaussian distribution, the median angle for each cell was selected for statistical analyses. These values were compared via two-tailed, nested t-test which revealed that the main processes of planar astrocytes had larger angles compared to the main processes of TE-RMS astrocytes (FIG. 10I; t=13.98, df=13, p<0.0001). All measured angles for both groups (n=1069 planar; n=644 TE-RMS) are also presented together (FIG. 10I) to display the apparent differences between planar and TE-RMS samples (statistical testing was not performed on FIG. 10I as the more appropriate arrangement for given assumptions was determined a priori to be the nested t-tests as shown previously). Whereas planar astrocyte process angles were fairly evenly spread between a 0 and 90 degree deviation from the long nuclear axis, TE-RMS astrocyte process angles clustered closer to 0 degrees with far fewer angles measuring close to a 90 degree deviation. The larger the angle measurement, the further the main process deviates from the long nuclear axis. Whereas planar astrocytes generally extended processes in all directions (FIG. 10B, 10C), TE-RMS astrocytes generally extended “bidirectional” processes in only two directions parallel with the long nuclear axis of the cell (FIGS. 10E, 10F).

Endogenous Protoplasmic Astrocytes have a More Complex and Varied Cytoskeletal Arrangement Compared to Endogenous RMS Astrocytes in Rat Brain

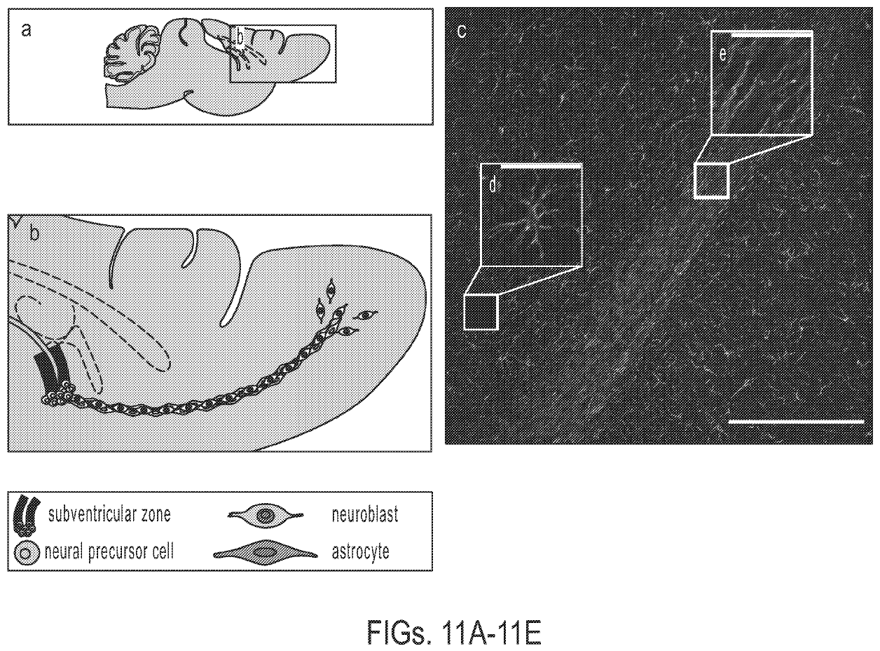

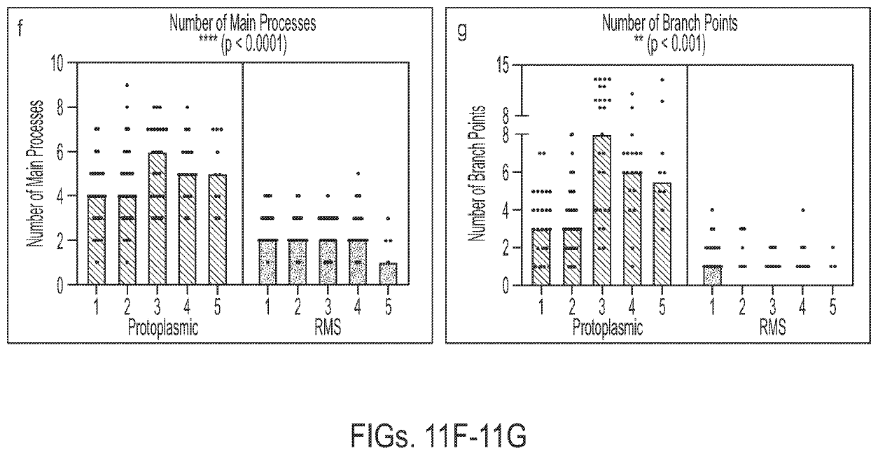

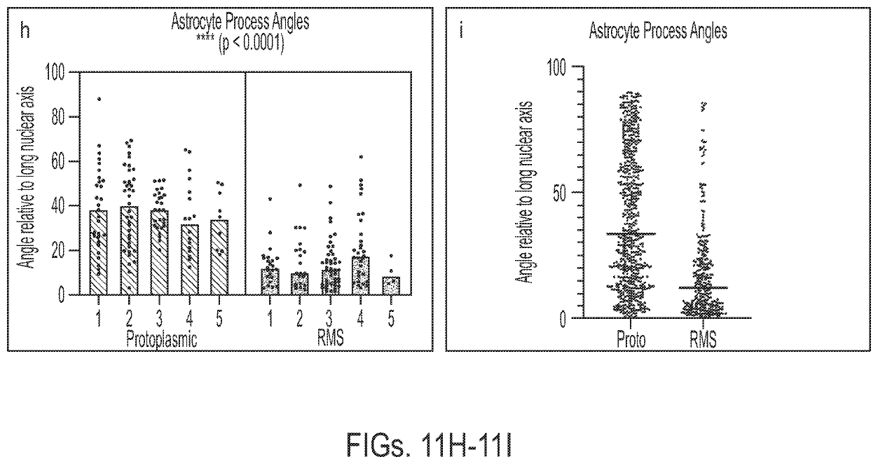

To compare our in vitro cytoskeletal findings to the rat brain in vivo, we applied fluorescence immunohistochemistry (IHC) techniques to label GFAP and Hoechst in sagittally sectioned (FIG. 11A) adult rat brains (n=5). For each subject, a single 20× fluorescent con-focal image that contained RMS and non-RMS tissue was used for quantification. SLike our in vitro measurements, we quantified the number of main processes, number of branch points, and angle of each main process across protoplasmic (non-RMS) astrocytes (n=124 cells) and RMS astrocytes (n=131 cells). All cells with visible, non-overlapping nuclei and clear GFAP process extensions within each image were analyzed. An example representative image (FIG. 11C) depicting the cytoskeleton of a single protoplasmic astrocyte (FIG. 11D) and several RMS astrocytes (FIG. 11E) reveal distinct cytoskeletal arrangement between these two cell populations. The number of main processes were compared by a two-tailed, nested t-test which revealed that protoplasmic astrocytes had a significantly higher number of main processes compared to RMS astrocytes (FIG. 11F; t=7.334, df=8, p<0.0001). The number of branch points were also compared by two-tailed, nested t-test which revealed that protoplasmic astrocytes had a significantly higher number of branch points compared to RMS astrocytes (FIG. 11G; t=4.732, df=8, p=0.0015). The angle of each main process was again measured relative to the long nuclear axis of the cell, and the median angle measurement for each cell was compared via two-tailed, nested t-test which revealed that the main processes of protoplasmic astrocytes had larger angles compared to the main processes of RMS astrocytes (FIG. 11H; t=10.09, df=8, p<0.0001). All measured angles (n=585 protoplasmic; n=303 RMS) were again displayed (FIG. 11I) to depict apparent differences between groups, and statistical analyses were not run on these values. The angles of protoplasmic astrocytes were evenly spread between 0 and 90 degrees, whereas the angles of RMS astrocytes clustered closer to 0 degrees. These data collectively demonstrate that endogenous protoplasmic astrocytes possess increased cytoskeletal complexity compared to astrocytes in the endogenous RMS. The increased cytoskeletal complexity of endogenous protoplasmic astrocytes compared to RMS astrocytes (FIGS. 11A-11I) mirrors the increased cytoskeletal complexity of planar astrocytes compared to TE-RMS astrocytes (FIGS. 10A-10J). In this way, the mechanical manipulation of astrocytes into the TE-RMSs caused their cytoskeletal complexity to mimic that of the endogenous rat RMS.

### TE-RMS Astrocytes Possess Elongated Nuclei Compared to Planar Astrocyte

### Sister Cultures

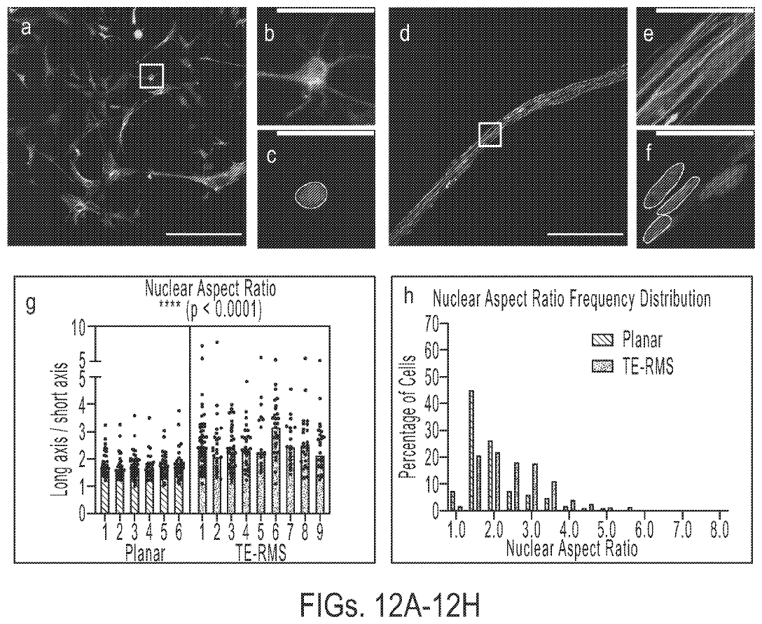

We performed nuclear measurements in the same planar (FIGS. 12A-12C) and TE-RMS (FIGS. 12D-12F) astrocytes that were used for cytoskeletal analyses described above (FIGS. 10A-10J). The long and short nuclear axes of each cell were measured, and the long nuclear axis was divided by the short nuclear axis to obtain the nuclear aspect ratio for each cell. Nu-clear aspect ratios were compared by a two-tailed, nested t-test which demonstrated that TE-RMS astrocytes had significantly higher nuclear aspect ratios compared to planar astrocytes (FIG. 12G; t=8.041, df=13, p<0.0001). A higher nuclear aspect ratio equates to a cell possessing a more elongated nucleus. These results indicate that TE-RMS astrocyte nuclei were significantly more elongated in shape whereas planar astrocyte nuclei were rounder. These differences in planar (FIG. 12C) and TE-RMS (FIG. 12F) nuclear shape are apparent even in the absence of quantification. Nuclear aspect ratio frequency distributions were plotted for planar and TE-RMS astrocytes (FIG. 12H). Statistics were not run on these distributions, but the evident difference in the shape of the distributions highlights the elongation of astrocyte nuclei in the TE-RMS compared to planar sister cultures. Whereas the majority (>50%) of planar astrocytes have a nuclear aspect ratio at or below 1.5, the majority (>70%) of TE-RMS astrocytes have nuclear aspect ratios greater than 1.5, with some TE-RMS astrocytes having a nuclear aspect ratio as high as 8.

### RMS Astrocytes Possess Elongated Nuclei Compared to Protoplasmic Astrocytes in the Endogenous Rat Brain

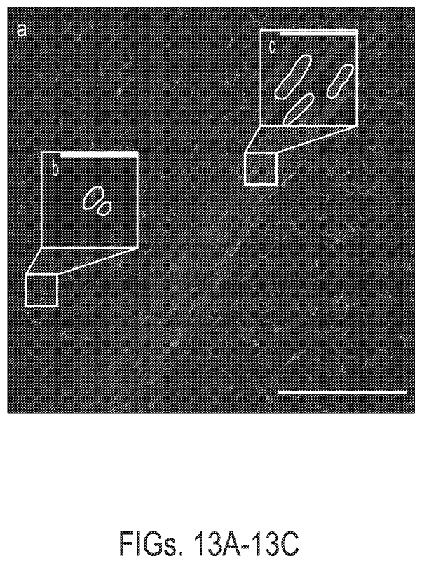

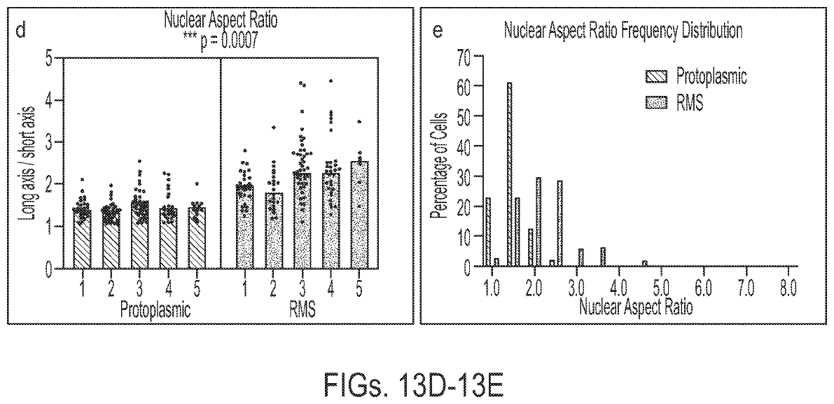

To compare our in vitro nuclear measurements to astrocytes in the endogenous rat brain, we performed nuclear measurements in the same endogenous protoplasmic and RMS astrocytes (FIGS. 13A-13C) that were used for in vivo cytoskeletal measurements (FIGS. 11A-11I). Nuclear aspect ratios were compared by a two-tailed, nested t-test. RMS astrocytes had significantly higher nuclear aspect ratios (i.e., significantly more elongated nuclei) com-pared to protoplasmic astrocytes (FIG. 13D; t=5.372, df=8, p=0.0007). To further high-light this difference, the nuclear aspect ratio frequency distributions were plotted for protoplasmic and RMS (FIG. 13E) astrocytes (statistics were not run on these distributions). Whereas most protoplasmic astrocytes (>60%) had nuclear aspect ratios at or below 1.5 (like in vitro planar astrocytes), most RMS astrocytes (>70%) had nuclear aspect ratios greater than 1.5 (like TE-RMS astrocytes). Additionally, the cell density of the endogenous RMS was so high that it often prevented us from discerning where one nucleus ended and the other began. We measured conservatively and therefore suspect that some endogenous RMS astrocytes have even higher nuclear aspect ratios than reported here.

### High Magnification Fluorescent Imaging Highlights Profound Differences in Nuclear Morphology and Cytoskeletal Arrangement Between Planar and TE-RMS Astrocytes

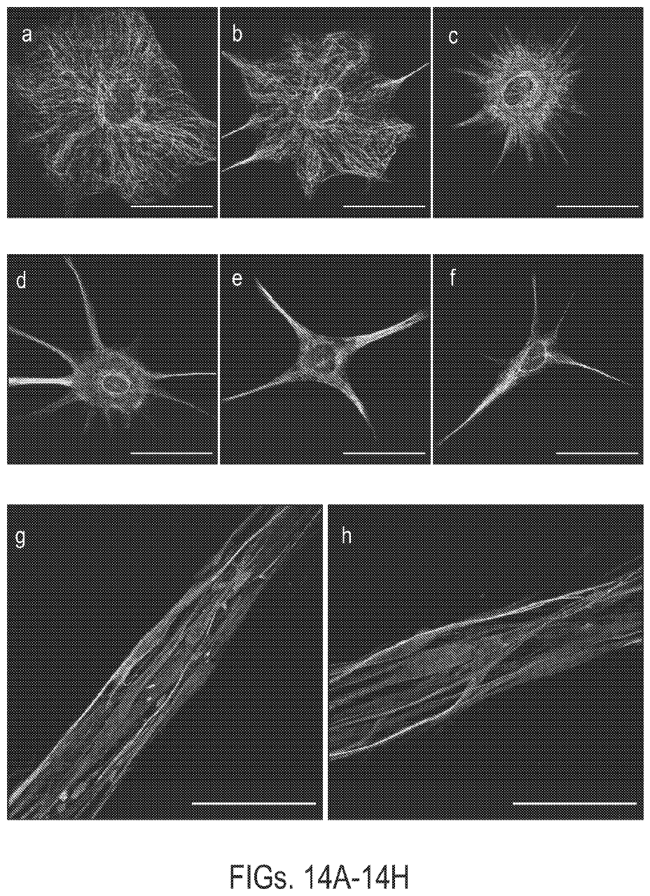

High magnification (100×) fluorescent confocal imaging was conducted to examine the differences between nuclear shape and intermediate filament arrangement of planar (FIGS. 14A-14F) and TE-RMS (FIGS. 14G, 14H) astrocytes in greater detail. FIGS. 14A-14H depicts the profound nuclear and cytoskeletal differences between astrocytes in these two different in vitro arrangements. Whereas planar astrocytes possess a round or slightly oblong nucleus, most TE-RMS astrocytes possess nuclei that are significantly elongated. Planar astrocytes exhibit complex, diverse arrangement of intermediate filaments. Whereas some planar astrocytes extend their intermediate filaments in numerous directions (FIGS. 14A, 14B), other planar astrocytes (FIGS. 14E, 14F) extend collections of intermediate filaments in only a few directions. On the other hand, most TE-RMS astrocytes extend intermediate filaments in two directions from opposite ends of the cell running parallel to the long nuclear axis and longitudinal axis of the construct.

### Scanning Electron Microscopy Imaging Confirms Novel Astrocytic Morphology of the TE-RMS

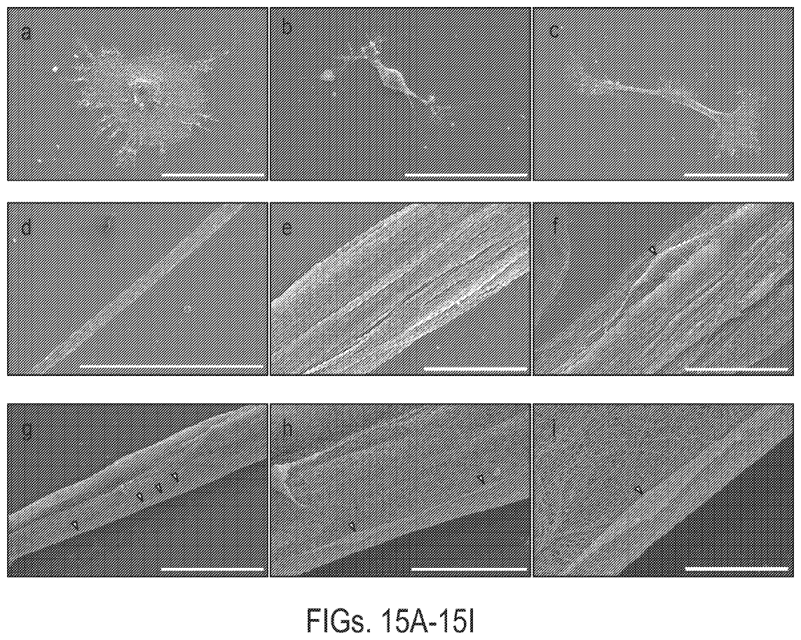

To further examine the fine structural differences in nuclear morphology and cytoskeletal arrangement, planar astrocytes (FIGS. 15A-15C) and TE-RMS astrocytes (FIG. 15D-15I) were imaged via SEM. SEM imaging readily reveals the complexity of processes arising from single astrocytes and the heterogeneity in planar astrocyte morphology. In contrast, TE-RMS astrocytes display bidirectional processes that are longitudinally aligned. Full-length TE-RMS (FIG. 15D) with zoom in (FIG. 15E) depicts this distinct arrangement of astrocyte processes in the TE-RMS compared to planar astrocytes (FIG. 15A-15C). Astrocyte bundles (FIG. 15E) are coated in a fine collagen meshwork from the polymerized collagen that astrocytes use to pull themselves together during TE-RMS formation. In many cases, the tightly aligned, bundled cell processes and collagen meshwork prevent most single cells from being distinguishable. However, some single TE-RMS astrocytes are visible under SEM (FIG. 15F). Some constructs (FIG. 15G) reveal several distinct astrocyte somata (black arrows) with visibly connecting processes. Magnified views of these cells (FIG. 15H, 15I) reveal the elongated somata and bidirectional processes of TE-RMS astrocytes as well as the interstitial meshwork of ultra-fine collagen fibrils.

### Comparison of TE-RMS and Endogenous Rat RMS Astrocytes

We also directly compared cytoskeletal and nuclear measurements of TE-RMS astrocytes and endogenous RMS astrocytes (data depicted in Table 1). Four two-tailed, nested t-tests were performed on measurements of main processes, number of branch points, angle of main processes, and nuclear aspect ratios. To further compare these parameters, we also present descriptive statistics to report the mean and standard deviation (SD). Here, mean±SD values for TE-RMS and RMS astrocytes were calculated by grouping all cells from each condition (Table 1). Degrees of freedom (DF), t values, and p values are from the nested t-test analyses run between TE-RMS and RMS groups. This testing showed that the number of main processes (t=0.6326, df=12, p=0.5388) and the number of branch points (t=0.1509, df=12, p=0.8825) were statistically equivalent between the TE-RMS and endogenous RMS astrocytes. However, there were modest differences in the angle of the main processes (t=2.861, df=12, p=0.0143) and the nuclear aspect ratio (t=2.490, df=12, p=0.0284) between astrocytes in the TE-RMS and astrocytes in the endogenous RMS. Interestingly, these analyses revealed that TE-RMS astrocytes were slightly more aligned and presented more oblong nuclei than endogenous RMS astrocytes.

TABLE 1
Quantitative comparison of nuclear shape and cytoskeletal
arrangement across TE-RMS and RMS astrocytes.
Nuclear and Cytoskeletal Comparison of TE-RMS and
Endogenous Rat RMS Astrocytes

| | TE-RMS Mean ± SD | RMS Mean ± SD | DF | T Value | p Value |
|---|---|---|---|---|---|
| Number of main processes | 2.35 ± 0.69 | 2.31 ± 0.98 | 12 | 0.6326 | 0.5388 |
| Number of branch points | 0.75 ± 0.98 | 0.78 ± 0.93 | 12 | 0.1509 | 0.8825 |
| Angle of main processes | 12.42 ± 16.42 | 18.63 ± 19.39 | 12 | 2.861 | 0.0143 |
| Nuclear aspect ratio | 2.56 ± 0.98 | 2.23 ± 0.67 | 12 | 2.490 | 0.0284 |

The TE-RMS is the first engineered biomimetic astrocytic microtissue that is designed to promote sustained neuroblast redirection into distal neuron-deficient brain regions. The TE-RMS may also be a useful platform to investigate the mechanisms of neuronal migration, cell-cell communication, and differentiation in vitro, which may also feature human cells to provide insights into human neurogenesis and migration. The self-assembly of the TE-RMS in biomaterial microcolumns leads to profound morphological changes in astrocytes. We demonstrate that when astrocytes are put into specific 3D conditions with all other conditions kept consistent, they undergo rapid process/cytoskeletal remodeling and nuclear elongation. We have previously demonstrated that a specific angle of curvature of hydrogel microcolumns is required for proper TE-RMS formation [26]. These unique bio-material conditions initiate a fundamentally different program in these cells that results in dramatic structural changes. These morphological features, which have not previously been described in astrocytes in vitro, do not require growth factors or media changes-rather, they are caused solely by the geometric and topographical feature of the 3D environment that the cells are exposed to. The resulting novel astrocytic phenotype warrants further investigation, for instance as a tool to investigate astrocyte cytoskeletal-nuclear dynamics and cell-environmental interactions that would otherwise not be possible and thereby expanding the capabilities of the TE-RMS as a tool for scientific discovery. Further, we demonstrate that the unique cytoskeletal arrangement and nuclear shape of TE-RMS astrocytes mimics that of astrocytes in the endogenous rat RMS. Whereas planar astrocyte cultures have similar morphology to endogenous protoplasmic astrocytes, the bidirectional processes and elongated nuclei that are characteristic of TE-RMS astrocytes mimic that of RMS astrocytes. These findings augment previous work demonstrating that the TE-RMS mimics the general structure and protein expression of the endogenous RMS, highlighting this technology as the first biomimetic astrocytic microtissue that replicates the structural and functional features of the endogenous pathway for neuroblast delivery in the mature brain.

While the number of main processes and branch points were equivalent between TE-RMS and endogenous RMS astrocytes, we reported small, yet statistically significant, differences in cytoskeletal alignment and nuclear shape measurements between TE-RMS and endogenous RMS astrocytes (as summarized in Table 1). The TE-RMS nuclear aspect ratio of 2.56±0.98 (mean±SD) was higher compared to that of the endogenous RMS nu-clear aspect ratio of 2.23±0.67 (mean±SD). Additionally, the main processes of TE-RMS astrocytes were more aligned with the longitudinal axes of the cells with a deviation of 12.42±16.42 (mean±SD) degrees compared to endogenous RMS astrocytes which had a deviation of 18.63±19.39 (mean±SD) degrees from the longitudinal axes. As these differences highlight, the phenomena of elongated nuclei and aligned cytoskeleton appear to be greater in the TE-RMS compared to that of in vivo RMS astrocytes. There are several possible explanations for these differences. First, our in vitro TE-RMS system is only around 24 h old at the time of these analyses. We predict that this system will continue to mature as it is cultured in vitro to further emulate in vivo RMS morphology. Additionally, our TE-RMS system lacks in vivo cues that could subtly affect astrocyte morphology, including the presence of vasculature and other cell types, the presence of various types of ECM and soluble factors, and signals from migrating neuroblasts.

Astrocytes are mechanosensitive, meaning that they can sense and respond to mechanical cues in their surrounding environment [35]. Recent research has begun to elucidate the mechanisms of astrocyte mechanosensation and the significance of this property for physiological astrocyte function in vivo [36,37]. Additionally, nuclear pore complexes, which fuse the two layers of the nuclear envelope, are mechanosensitive thereby making the nucleus itself sensitive to changes in cellular mechanical forces [38-40]. The four main contributing factors to the shape of a cell's nucleus are cytoskeletal forces, the thickness of the nuclear lamina, level of chromatin compaction, and activity of proteins that control chromatin conformation [41]. Here, we demonstrate significant nuclear elongation of astrocytes following TE-RMS formation, with some astrocytes having a nuclear aspect ratio as high as 8. This extent of TE-RMS nuclear elongation, which has never been reported in planar astrocyte cultures but mimics astrocyte morphology in the endogenous RMS, hap-pens remarkably quickly following astrocyte introduction to 3D conditions. The unique cytoskeletal changes that are concomitant with nuclear elongation during TE-RMS formation indicates that the cytoskeleton is involved in executing these nuclear morphological changes. Cytoskeletal forces propagate to the nucleus through the linker of nucleoskeleton and cytoskeleton (LINC) complex which connects the cytoskeleton to the nuclear envelope [41]. The LINC complex directly contacts cytoskeletal actin filaments and indirectly contacts intermediate filaments through cyto-linker proteins and microtubules through motor proteins to allow for mechanotransduction of extracellular forces to the nucleus [41]. Cytoskeletal forces regulate nuclear size, shape, orientation, and movement within a cell [41]. For example, Versaevel and colleagues demonstrated that the shape and orientation of endothelial cell nuclei can be controlled by compressive forces exerted by actin filaments [42]. Topographical curvature can also control the shape of epithelial cells in vitro, including the orientation and distribution of epithelial cell nuclei and F-actin [43]. Additionally, intra-ocular pressure has been shown to modulate the astrocyte cytoskeleton and nuclear shape in the optic nerve of mice [44]. In the TE-RMS, astrocyte processes align in parallel inside of microcolumns, and they work in conjunction with each other to stretch out the entire cell including the nucleus which can sense these mechanical changes. As cells stretch, the forces acting upon the nuclei become different than the forces acting upon cells in 2D conditions that have radial processes. While we hypothesize that cytoskeletal forces are the predominant mechanism leading to greater nuclear eccentricity, there could be additional factors that are causing these nuclear shape changes. For example, Kalinin and colleagues recently demonstrated that modulation of chromatin compaction with valproic acid treatment leads to changes in astrocyte nuclear shape [45]. We are currently investigating how these biomaterial conditions that induce TE-RMS formation affect the structure of the astrocyte nuclear lamina and chromatin arrangement to elucidate how they may contribute to astrocyte nuclear elongation.

Advanced imaging technologies provide an in-depth view of the astrocyte structural changes that result from TE-RMS formation. High magnification fluorescent imaging (FIGS. 14A-14H) clearly demonstrates the alignment of astrocyte processes and the extent of nu-clear elongation that is possible within this biomaterial conformation. The morphological heterogeneity of planar astrocytes is also evident, emphasizing and explaining the variability seen in main processes, branch points, and angle measurements in planar samples. Whereas planar astrocytes are quite morphologically diverse, TE-RMS astrocytes are mostly—but not exclusively—homogeneous in their morphology. SEM imaging (FIGS. 15A-15I) allows us to see the interaction of TE-RMS astrocytes and polymerized collagen that makes formation of these constructs possible. Individual collagen fibrils (which are visible under SEM) have a diameter between 10-500 nm with an average diameter of 40-80 nm [46,47], which is the size of the mesh of small fibers coating the TE-RMS constructs (FIGS. 15D-15I). It appears that the larger bundles (FIG. 15E) are astrocyte processes that are bundled and aligned in parallel and coated with this polymerized collagen network. While the specific structure of most cells is obscured beneath this layer of collagen, there are select cells that emerge from underneath the collagen layer and offer a detailed look at TE-RMS astrocyte morphology (FIGS. 15F-15I). The high resolution provided by SEM imaging pro-vides an insightful view of cell-cell contact between TE-RMS astrocytes. The processes of cells are aligned and interconnected to such an extent that it is difficult to discern where one cell ends and another begins (FIG. 15h). This starkly contrasts with planar astrocytes that form distinct, discernable domains. Astrocytes engage in cell-cell communication via gap junctions [48,49], whereas other cell types including myoblasts can communicate via cytoplasmic fusion [50]. Although the phenomenon of cytoplasmic fusion has not to our knowledge been reported in astrocytes, advanced imaging techniques utilized here indicate that this phenomenon could be possible in TE-RMS astrocytes. We are currently investigating the potential for astrocyte cytoplasmic fusion upon TE-RMS formation.

Herein, we reveal critical details describing the unique astrocyte structure that emerges from exposure to specific 3D biomaterial conditions. It is well known that mechanical changes in nuclear morphology are often accompanied by changes in chromatin organization and gene expression which then lead to downstream alterations in cell activity and signaling [41,51-54]. However, there is limited research on how mechanically altering nuclear shape affects gene expression regulation and cell behavior in astrocytes specifically [44,45]. Overall, we performed a thorough structural evaluation of the intermediate filament cytoskeleton and nuclear morphology in TE-RMS astrocytes, revealing a novel astrocyte phenotype that has not previously been described in vitro. The TE-RMS offers a powerful platform to investigate critical relationships between biomaterial surface cues and astrocyte behavior and function.

### Example 2 Cited Documents

1. Ming, G.- L.; Song, H. Adult Neurogenesis in the Mammalian Brain: Significant Answers and Significant Questions. Neuron 2011, 70, 687-702. https://doi.org/10.1016/j.neuron.2011.05.001.
2. Zhao, C.; Deng, W.; Gage, F. H. Mechanisms and Functional Implications of Adult Neurogenesis. Cell 2008, 132, 645-660. https://doi.org/10.1016/j.cell.2008.01.033.
3. Lledo, P.- M.; Merkle, F. T.; Alvarez-Buylla, A. Origin and Function of Olfactory Bulb Interneuron Diversity. Trends Neurosci. 2008, 31, 392-400. https://doi.org/10.1016/j.tins.2008.05.006.
4. Lim, D. A.; Alvarez-Buylla, A. The Adult Ventricular-Subventricular Zone (V-SVZ) and Olfactory Bulb (OB) Neurogenesis. Cold Spring Harb. Perspect. Biol. 2016, 8, a018820, https://doi.org/10.1101/cshperspect.a018820.
5. Lazarini, F.; Lledo, P.- M. Is Adult Neurogenesis Essential for Olfaction? Trends Neurosci. 2011, 34, 20-30. https://doi.org/10.1016/j.tins.2010.09.006.
6. Brill, M. S.; Ninkovic, J.; Winpenny, E.; Hodge, R. D.; Ozen, I.; Yang, R.; Lepier, A.; Gascón, S.; Erdelyi, F.; Szabo, G.; et al. Adult Generation of Glutamatergic Olfactory Bulb Interneurons. Nat. Neurosci. 2009, 12, 1524-1533. https://doi.org/10.1038/nn.2416.
7. Lu, J.; Manaenko, A.; Hu, Q. Targeting Adult Neurogenesis for Poststroke Therapy. Stem Cells Int. 2017, 2017, 5868632. https://doi.org/10.1155/2017/5868632.
8. Tang, H.; Wang, Y.; Xie, L.; Mao, X.; Won, S. J.; Galvan, V.; Jin, K. Effect of Neural Precursor Proliferation Level on Neurogenesis in Rat Brain during Aging and after Focal Ischemia. Neurobiol. Aging 2009, 30, 299-308. https://doi.org/10.1016/j.neurobiolaging.2007.06.004.
9. Chang, E. H.; Adorjan, I.; Mundim, M. V.; Sun, B.; Dizon, M. L. V.; Szele, F. G. Traumatic Brain Injury Activation of the Adult Subventricular Zone Neurogenic Niche. Front. Neurosci. 2016, 10, 332. https://doi.org/10.3389/fnins.2016.00332.
10. Kaneko, N.; Herranz-Pérez, V.; Otsuka, T.; Sano, H.; Ohno, N.; Omata, T.; Nguyen, H. B.; Thai, T. Q.; Nambu, A.; Kawaguchi, Y.; et al. New Neurons Use Slit-Robo Signaling to Migrate through the Glial Meshwork and Approach a Lesion for Functional Regeneration. Sci. Adv. 2018, 4, eaav0618. https://doi.org/10.1126/sciadv.aav0618.
11. Hou, S.- W.; Wang, Y.- Q.; Xu, M.; Shen, D.- H.; Wang, J.- J.; Huang, F.; Yu, Z.; Sun, F.-Y. Functional Integration of Newly Generated Neurons into Striatum after Cerebral Ischemia in the Adult Rat Brain. Stroke 2008, 39, 2837-2844. https://doi.org/10.1161/STROKEAHA. 107.510982.
12. Wang, Z.; Zheng, Y.; Zheng, M.; Zhong, J.; Ma, F.; Zhou, B.; Zhu, J. Neurogenic Niche Conversion Strategy Induces Migration and Functional Neuronal Differentiation of Neural Precursor Cells Following Brain Injury. Stem Cells Dev. 2020, 29, 235-248, scd.2019.0147. https://doi.org/10.1089/scd.2019.0147.
13. Ohab, J. J.; Fleming, S.; Blesch, A.; Carmichael, S. T. A Neurovascular Niche for Neurogenesis after Stroke. J. Neurosci. 2006, 26, 13007-13016. https://doi.org/10.1523/JNEUROSCI.4323-06.2006.
14. Kolb, B.; Morshead, C.; Gonzalez, C.; Kim, M.; Gregg, C.; Shingo, T.; Weiss, S. Growth Factor-Stimulated Generation of New Cortical Tissue and Functional Recovery after Stroke Damage to the Motor Cortex of Rats. J. Cereb. Blood Flow Metab. 2007, 27, 983-997. https://doi.org/10.1038/sj.jcbfm.9600402.
15. Schäbitz, W.- R.; Steigleder, T.; Cooper-Kuhn, C. M.; Schwab, S.; Sommer, C.; Schneider, A.; Kuhn, H. G. Intravenous Brain-Derived Neurotrophic Factor Enhances Poststroke Sensorimotor Recovery and Stimulates Neurogenesis. Stroke 2007, 38, 2165-2172. https://doi.org/10.1161/STROKEAHA. 106.477331.
16. Jinnou, H.; Sawada, M.; Kawase, K.; Kaneko, N.; Herranz-Pérez, V.; Miyamoto, T.; Kawaue, T.; Miyata, T.; Tabata, Y.; Akaike, T.; et al. Radial Glial Fibers Promote Neuronal Migration and Functional Recovery after Neonatal Brain Injury. Cell Stem Cell 2018, 22, 128-137.e9. https://doi.org/10.1016/j.stem.2017.11.005.
17. Gundelach, J.; Koch, M. Redirection of Neuroblast Migration from the Rostral Migratory Stream into a Lesion in the Prefrontal Cortex of Adult Rats. Exp. Brain Res. 2018, 236, 1181-1191. https://doi.org/10.1007/s00221-018-5209-3.
18. Clark, A. R.; Carter, A. B.; Hager, L. E.; Price, E. M. In Vivo Neural Tissue Engineering: Cylindrical Biocompatible Hydrogels That Create New Neural Tracts in the Adult Mammalian Brain. Stem Cells Dev. 2016, 25, 1109-1118. https://doi.org/10.1089/scd.2016.0069.
19. Yu, S.- J.; Tseng, K.- Y.; Shen, H.; Harvey, B. K.; Airavaara, M.; Wang, Y. Local Administration of AAV-BDNF to Subventricular Zone Induces Functional Recovery in Stroke Rats. PLOS ONE 2013, 8, e81750. https://doi.org/10.1371/journal.pone.0081750.
20. Erlandsson, A.; Lin, C.- H. A.; Yu, F.; Morshead, C. M. Immunosuppression Promotes Endogenous Neural Stem and Progenitor Cell Migration and Tissue Regeneration after Ischemic Injury. Exp. Neurol. 2011, 230, 48-57. https://doi.org/10.1016/j.expneurol.2010.05.018.
21 Petraglia, A. L.; Marky, A. H.; Walker, C.; Thiyagarajan, M.; Zlokovic, B. V. Activated Protein C Is Neuroprotective and Mediates New Blood Vessel Formation and Neurogenesis after Controlled Cortical Impact. Neurosurgery 2010, 66, 165-171; discussion 171-2. https://doi.org/10.1227/01.NEU.0000363148.49779.68.
22. Popa-Wagner, A.; Stöcker, K.; Balseanu, A. T.; Rogalewski, A.; Diederich, K.; Minnerup, J.; Margaritescu, C.; Schäbitz, W.- R. Effects of Granulocyte-Colony Stimulating Factor After Stroke in Aged Rats. Stroke 2010, 41, 1027-1031. https://doi.org/10.1161/STROKEAHA. 109.575621.
23. Purvis, E. M.; O'Donnell, J. C.; Chen, H. I.; Cullen, D. K. Tissue Engineering and Biomaterial Strategies to Elicit Endogenous Neuronal Replacement in the Brain. Front. Neurol. 2020, 11, 344, https://doi.org/10.3389/fneur.2020.00344
24. Oliveira, E. P.; Silva-Correia, J.; Reis, R. L.; Oliveira, J. M. Biomaterials Developments for Brain Tissue Engineering. Adv. Exp. Med. Biol. 2018, 1078, 323-346. https://doi.org/10.1007/978-981-13-0950-2_17.
25 Pettikiriarachchi, J. T. S.; Parish, C. L.; Shoichet, M. S.; Forsythe, J. S.; Nisbet, D. R. Biomaterials for Brain Tissue Engineering. Aust. J. Chem. 2010, 63, 1143. https://doi.org/10.1071/CH10159.
26. Winter, C. C.; Katiyar, K. S.; Hernandez, N. S.; Song, Y. J.; Struzyna, L. A.; Harris, J. P.; Cullen, D. K. Transplantable Living Scaffolds Comprised of Micro-Tissue Engineered Aligned Astrocyte Networks to Facilitate Central Nervous System Regeneration. Acta Biomater. 2016, 38, 44-58. https://doi.org/10.1016/j.actbio.2016.04.021.
27. Katiyar, K. S.; Winter, C. C.; Gordian-Vélez, W. J.; O'Donnell, J. C.; Song, Y. J.; Hernandez, N. S.; Struzyna, L. A.; Cullen, D. K. Three-Dimensional Tissue Engineered Aligned Astrocyte Networks to Recapitulate Developmental Mechanisms and Facilitate Nervous System Regeneration. J. Vis. Exp. 2018, 55848. https://doi.org/10.3791/55848.
28. O'Donnell, J.; Katiyar, K.; Panzer, K.; Cullen, Dk. A Tissue-Engineered Rostral Migratory Stream for Directed Neuronal Replacement. Neural Regen. Res. 2018, 13, 1327. https://doi.org/10.4103/1673-5374.235215.
29. O'Donnell, J. C.; Purvis, E. M.; Helm, K. V. T.; Adewole, D. O.; Zhang, Q.; Le, A. D.; Cullen, D. K. An Implantable Human Stem Cell-Derived Tissue-Engineered Rostral Migratory Stream for Directed Neuronal Replacement. Commun. Biol. 2021, 4, 879. https://doi.org/10.1038/s42003-021-02392-8.
30. Struzyna, L. A.; Adewole, D. O.; Gordián-Vélez, W. J.; Grovola, M. R.; Burrell, J. C.; Katiyar, K. S.; Petrov, D.; Harris, J. P.; Cullen, D. K. Anatomically Inspired Three-Dimensional Micro-Tissue Engineered Neural Networks for Nervous System Reconstruction, Modulation, and Modeling. J. Vis. Exp. 2017, e55609. https://doi.org/10.3791/55609.
31. Struzyna, L. A.; Browne, K. D.; Brodnik, Z. D.; Burrell, J. C.; Harris, J. P.; Chen, H. I.; Wolf, J. A.; Panzer, K. V.; Lim, J.; Duda, J. E.; et al. Tissue Engineered Nigrostriatal Pathway for Treatment of Parkinson's Disease. J. Tissue Eng. Regen. Med. 2018, 12, 1702-1716. https://doi.org/10.1002/term.2698.
32. Braet, F.; De Zanger, R.; Wisse, E. Drying Cells for SEM, AFM and TEM by Hexamethyldisilazane: A Study on Hepatic Endothelial Cells. J. Microsc. 1997, 186, 84-87. https://doi.org/10.1046/j.1365-2818.1997.1940755.x.
33. Schindelin, J.; Arganda-Carreras, I.; Frise, E.; Kaynig, V.; Longair, M.; Pietzsch, T.; Preibisch, S.; Rueden, C.; Saalfeld, S.; Schmid, B.; et al. Fiji: An Open-Source Platform for Biological-Image Analysis. Nat. Methods 2012, 9, 676-682. https://doi.org/10.1038/nmeth.2019.
34. Peretto, P.; Merighi, A.; Fasolo, A.; Bonfanti, L. Glial Tubes in the Rostral Migratory Stream of the Adult Rat. Brain Res. Bull. 1997, 42, 9-21. https://doi.org/10.1016/S0361-9230(96)00116-5.
35 Moshayedi, P.; da F Costa, L.; Christ, A.; Lacour, S. P.; Fawcett, J.; Guck, J.; Franze, K. Mechanosensitivity of Astrocytes on Optimized Polyacrylamide Gels Analyzed by Quantitative Morphometry. J. Phys. Condens. Matter 2010, 22, 194114. https://doi.org/10.1088/0953-8984/22/19/194114.
36 Turovsky, E. A.; Braga, A.; Yu, Y.; Esteras, N.; Korsak, A.; Theparambil, S. M.; Hadjihambi, A.; Hosford, P. S.; Teschemacher, A. G.; Marina, N.; et al. Mechanosensory Signaling in Astrocytes. J. Neurosci. 2020, 40, 9364-9371. https://doi.org/10.1523/JNEUROSCI.1249-20.2020.
37. Marina, N.; Christie, I. N.; Korsak, A.; Doronin, M.; Brazhe, A.; Hosford, P. S.; Wells, J. A.; Sheikhbahaei, S.; Humoud, I.; Paton, J. F. R.; et al. Astrocytes Monitor Cerebral Perfusion and Control Systemic Circulation to Maintain Brain Blood Flow. Nat. Commun. 2020, 11, 131. https://doi.org/10.1038/s41467-019-13956-y.
38. Kirby, T. J.; Lammerding, J. Emerging Views of the Nucleus as a Cellular Mechanosensor. Nat. Cell Biol. 2018, 20, 373-381. https://doi.org/10.1038/s41556-018-0038-y.
39 Graham, D. M.; Burridge, K. Mechanotransduction and Nuclear Function. Curr. Opin. Cell Biol. 2016, 40, 98-105. https://doi.org/10.1016/j.ceb.2016.03.006.
40. Zimmerli, C. E.; Allegretti, M.; Rantos, V.; Goetz, S. K.; Obarska-Kosinska, A.; Zagoriy, I.; Halavatyi, A.; Hummer, G.; Mahamid, J.; Kosinski, J.; et al. Nuclear Pores Dilate and Constrict in Cellulo. Science 2021, 374, eabd9776. https://doi.org/10.1126/science.abd9776.
41. dos Santos, A.; Toseland, C. P. Regulation of Nuclear Mechanics and the Impact on DNA Damage. Int. J. Mol. Sci. 2021, 22, 3178. https://doi.org/10.3390/ijms22063178.
42. Versaevel, M.; Grevesse, T.; Gabriele, S. Spatial Coordination between Cell and Nuclear Shape within Micropatterned Endothelial Cells. Nat. Commun. 2012, 3, 671. https://doi.org/10.1038/ncomms1668.
43. Rougerie, P.; Pieuchot, L.; dos Santos, R. S.; Marteau, J.; Bigerelle, M.; Chauvy, P.- F.; Farina, M.; Anselme, K. Topographical Curvature Is Sufficient to Control Epithelium Elongation. Sci. Rep. 2020, 10, 14784. https://doi.org/10.1038/s41598-020-70907-0.
44. Ling, Y. T. T.; Pease, M. E.; Jefferys, J. L.; Kimball, E. C.; Quigley, H. A.; Nguyen, T. D. Pressure-Induced Changes in Astrocyte GFAP, Actin, and Nuclear Morphology in Mouse Optic Nerve. Investig. Opthalmol. Vis. Sci. 2020, 61, 14. https://doi.org/10.1167/iovs.61.11.14.
45. Kalinin, A. A.; Hou, X.; Ade, A. S.; Fon, G.- V.; Meixner, W.; Higgins, G. A.; Sexton, J. Z.; Wan, X.; Dinov, I. D.; O'Meara, M. J.; et al. Valproic Acid-Induced Changes of 4D Nuclear Morphology in Astrocyte Cells. Mol. Biol. Cell 2021, 32, mbc.E20-08-0502. https://doi.org/10.1091/mbc.E20-08-0502.
46 USHIKI, T. Collagen Fibers, Reticular Fibers and Elastic Fibers. A Comprehensive Understanding from a Morphological Viewpoint. Arch. Histol. Cytol. 2002, 65, 109-126. https://doi.org/10.1679/aohc.65.109.
47. Shoulders, M. D.; Raines, R. T. Collagen Structure and Stability. Annu. Rev. Biochem. 2009, 78, 929-958. https://doi.org/10.1146/annurev.biochem.77.032207.120833.
48. Orellana, J. A.; Martinez, A. D.; Retamal, M. A. Gap Junction Channels and Hemichannels in the CNS: Regulation by Signaling Molecules. Neuropharmacology 2013, 75, 567-582. https://doi.org/10.1016/j.neuropharm.2013.02.020.
49 Finkbeiner, S. Calcium Waves in Astrocytes-Filling in the Gaps. Neuron 1992, 8, 1101-1108. https://doi.org/10.1016/0896-6273(92)90131-V.
50. Petrany, M. J.; Millay, D. P. Cell Fusion: Merging Membranes and Making Muscle. Trends Cell Biol. 2019, 29, 964-973. https://doi.org/10.1016/j.tcb.2019.09.002.
51 Haftbaradaran Esfahani, P.; Knöll, R. Cell Shape: Effects on Gene Expression and Signaling. Biophys. Rev. 2020, 12, 895-901. https://doi.org/10.1007/s12551-020-00722-4.
52. Seelbinder, B.; Ghosh, S.; Schneider, S. E.; Scott, A. K.; Berman, A. G.; Goergen, C. J.; Margulies, K. B.; Bedi, K. C.; Casas, E.; Swearingen, A. R.; et al. Nuclear Deformation Guides Chromatin Reorganization in Cardiac Development and Disease. Nat. Biomed. Eng. 2021, 5, 1500-1516. https://doi.org/10.1038/s41551-021-00823-9.
53 Ramdas, N. M.; Shivashankar, G. V. Cytoskeletal Control of Nuclear Morphology and Chromatin Organization. J. Mol. Biol. 2015, 427, 695-706. https://doi.org/10.1016/j.jmb.2014.09.008.
54. Skinner, B. M.; Johnson, E. E. P. Nuclear Morphologies: Their Diversity and Functional Relevance. Chromosoma 2017, 126, 195-212. https://doi.org/10.1007/s00412-016-0614-5.

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

1. A method of obtaining at least one astrocyte, the method comprising:
(a) providing at least one gingiva-derived mesenchymal stem cell (GDMSC), and
(b) contacting at least one GDMSC with:
(i) a first serum-free medium comprising N-2 supplement, basic fibroblast growth factor 2 (bFGF) and epidermal growth factor (EGF); and
(ii) a second serum-free medium comprising: dibutyryl cyclic adenosine monophosphate (dbcAMP), IBMX (3-Isobutyl-1-methylxanthine), neuregulin, and platelet-derived growth factor (PDGF)
wherein the GDMSC is contacted with a first medium and/or a second medium under conditions sufficient to induce differentiation of the GDMSC into an astrocyte.

2. The method of claim 1, wherein the GDMSC is contacted with the first medium for about 72 hours.

3. The method of claim 1, wherein the GDMSC is contacted with the second medium for about 72 hours.

4. The method of claim 1, wherein the first medium and/or the second medium further comprises one or more additional components.

5. The method of claim 4, wherein the one or more additional components comprises: an antibiotic, an amino acid supplement, an anti-fungal, or a combination thereof.

6. The method of claim 1, wherein the first medium comprises about 20 ng/ml of N-2 supplement, about 20 ng/ml basic fibroblast growth factor 2 (bFGF) and about 20 ng/ml epidermal growth factor (EGF).

7. The method of claim 1, wherein the second medium comprises about 0.5 to 1 mM dibutyryl cyclic adenosine monophosphate (dbcAMP), about 0.5 to 1 mM IBMX (3-Isobutyl-1-methylxanthine), about 50 ng/ml to 100 ng/ml of neuregulin, and about 1 ng/ml to 5 ng/ml of platelet-derived growth factor (PDGF).

8. The method of claim 1, wherein the GDMSC comprises one or more of the following characteristics:
(i) expression of Oct4, SSEA-4, Stro-1, CD29, CD73, CD90, CD105, Type I collagen, or a combination thereof;
(ii) has low or no detectable expression of CD45;
(iii) is capable of forming colonies in a colony formation assay;
(iv) is capable of differentiating into one or more progenitor cells such as adipocytes and osteoblasts;
(v) is capable of suppressing proliferation of T cells; and/or
(vi) is capable of suppressing proliferation of T cells by expressing one or more soluble mediators such as IDO and IL-10.

9. The method of claim 1, wherein the amount of time required to obtain an astrocyte is reduced relative to a comparator.

10. The method of claim 9, wherein a comparator comprises an otherwise similar method using induced pluripotent stem cells (iPSC) to obtain an astrocyte.

11. The method of claim 9, wherein the amount of time required to obtain an astrocyte is less than 1 week, less than 6 days, less than 5 days, or less than 4 days.

12. The method of claim 1, wherein an astrocyte obtained from the method comprises one or more of the following characteristics:
(i) expresses GFAP, Ezrin, Robo2, S-100-beta, glutamine synthetase (GS) and/or Glutamate Aspartate Transporter (GLAST);
(ii) expresses pyruvate carboxylase (PC) and/or Glutamate transporter-1 (GLT-1);
(iii) has low or no expression of an endothelial marker, e.g., CD31;
(iv) is capable of self-assembly into a bundle of longitudinally-aligned astrocytes with bipolar or multipolar processes,
(v) is capable of forming a bundle of astrocytes; and/or
(vi) has a morphology of an astrocyte.

13. The method of claim 12, wherein a bundle of astrocytes comprises a structure similar or substantially similar to a structure of an astrocyte in an endogenous rostral migratory stream.

14. The method of claim 13, wherein a bundle of astrocytes comprises a function similar to or substantially similar to a function of an astrocyte in an endogenous rostral migratory stream.

15. The method of claim 1, wherein the GDMSC is obtained from a population of cells in which at least 75% of the population of cells are cranial neural crest derived mesenchymal stem cells and/or no more than 25% of the population of cells are mesoderm derived mesenchymal stem cells.

16. The method of claim 15, wherein the neural crest derived mesenchymal stem cells:
(i) have an increased ability to differentiate into neuronal cells as compared to mesoderm derived mesenchymal stem cells; and/or
(ii) have an increased immunomodulatory capacity compared to mesoderm derived mesenchymal stem cells, optionally wherein, the immunomodulatory capacity comprises one or more of: (a) an increase in number and/or activity of T regulatory cells; (b) a decrease in number and/or activity of inflammatory T cells such as Th17 cells; or (c) an increase in number and/or activity of cytotoxic T cells.

17. A reaction mixture comprising at least one GDMSC and a serum-free medium comprising N-2 supplement, basic fibroblast growth factor 2 (bFGF) and epidermal growth factor (EGF).

18. The reaction mixture of claim 17, further comprising one or more additional components.

19. The reaction mixture of claim 18, wherein the one or more additional components comprises: an antibiotic, an amino acid supplement, an anti-fungal, or a combination thereof.

20. A reaction mixture comprising at least one GDMSC and a medium comprising: dibutyryl cyclic adenosine monophosphate (dbcAMP), IBMX (3-Isobutyl-1-methylxanthine), neuregulin, and platelet-derived growth factor (PDGF).

21. The reaction mixture of claim 20, further comprising one or more additional components.

22. The reaction mixture of claim 21, wherein the one or more additional components comprises: an antibiotic, an amino acid supplement, an anti-fungal, or a combination thereof.

23. The reaction mixture of claim 20, wherein the reaction mixture is maintained under conditions sufficient to obtain an astrocyte.

24. The reaction mixture of claim 23, wherein an astrocyte obtained from the reaction mixture comprises one or more of the following characteristics:
(i) expresses GFAP, Ezrin, Robo2, S-100-beta, glutamine synthetase (GS) and/or GLutamate ASpartate Transporter (GLAST);
(ii) expresses pyruvate carboxylase (PC) and/or Glutamate transporter-1 (GLT-1);
(iii) has low or no expression of an endothelial marker;
(iv) is capable of self-assembly into a bundle of longitudinally-aligned astrocytes with bipolar or multipolar processes;
(v) is capable of forming a bundle of astrocytes; and/or
(vi) has a morphology of an astrocyte.

25. The reaction mixture of claim 24, wherein a bundle of astrocytes obtained from the reaction mixture comprises a structure similar to or substantially similar to a structure of an astrocyte in an endogenous rostral migratory stream.

26. The reaction mixture of claim 24, wherein a bundle of astrocytes obtained from the reaction mixture comprises a function similar to or substantially similar to a function of an astrocyte in an endogenous rostral migratory stream.

27. A system, comprising:
(a) a plurality of astrocytes derived from at least one gingiva-derived mesenchymal stem cell (GDMSC); and
(b) a biocompatible construct comprising a matrix and having a first end, a second end and a body.

28. (canceled)

29. The system of claim 27, wherein a GDMSC comprises one or more of the following characteristics:
(i) express Oct4, SSEA-4, Stro-1, CD29, CD73, CD90, CD105, Type I collagen, or a combination thereof;
(ii) has low or no detectable expression of CD45;
(iii) is capable of forming colonies in a colony formation assay;
(iv) is capable of differentiating into one or more progenitor cells such as adipocytes and osteoblasts;
(v) is capable of suppressing proliferation of T cells; and/or
(vi) is capable of suppressing proliferation of T cells by expressing one or more soluble mediators such as IDO and IL-10.

30. The system of claim 27, wherein the GDMSC is obtained from a population of cells in which at least 75% of the population of cells are cranial neural crest derived mesenchymal stem cells and/or no more than 25% of the population of cells are mesoderm derived mesenchymal stem cells.

31. The system of claim 30, wherein the neural crest derived mesenchymal stem cells:
(i) have an increased ability to differentiate into neuronal cells as compared to mesoderm derived mesenchymal stem cells; and/or
(ii) have an increased immunomodulatory capacity compared to mesoderm derived mesenchymal stem cells.

32. The system of claim 27, wherein an astrocyte derived from a GDMSC comprises one or more of the following characteristics:
(i) expresses GFAP, Ezrin, Robo2, S-100-beta, glutamine synthetase (GS) and/or Glutamate Aspartate Transporter (GLAST);
(ii) expresses pyruvate carboxylase (PC) and/or Glutamate transporter-1 (GLT-1);
(iii) has low or no expression of an endothelial marker;
(iv) is capable of self-assembly into a bundle of longitudinally-aligned astrocytes with bipolar or multipolar processes;
(v) is capable of forming a bundle of astrocytes; and/or
(vi) has a morphology of an astrocyte.

33. The system of claim 27, further comprising a Slit-Robo entity.

34. The system of claim 33, wherein the Slit-Robo entity is or comprises an agent that promotes activation and/or signaling from a Slit-Robo pathway.

35. The system of claim 27, characterized in that when implanted into an organism, it promotes migration of one or more cells compared to a comparator.

36. The system of claim 35, wherein a comparator comprises an otherwise similar system without a plurality of astrocytes; or without a plurality of astrocytes derived from at least one GDMSC.

37. The system of claim 35, wherein one or more migrating cells comprises one or more endogenous host cells.

38. The system of claim 37, wherein an endogenous host cell comprises a neural precursor cell, a neuroblast, a neuron, a progenitor cell, a glial cell, an astrocyte, and/or an endothelial cell.

39. The system of claim 35, wherein one or more migrating cells comprises: one or more cells provided in the system; or one or more cells derived from, differentiated from or a progenitor of a cell provided in the system.

40. The system of claim 35, wherein migration of one or more cells occurs within the system, throughout the system, out of the system or into the system.

41. The system of claim 35, wherein migration of one or more cells occurs to the site of implantation, or away from the site of implantation.

42. The system of claim 27, wherein a matrix of a biocompatible construct comprises an inner surface and an outer surface.

43. The system of claim 42, wherein an inner surface of the biocompatible construct defines a luminal core.

44. The system of claim 42, wherein an outer surface of the biocompatible construct comprises at least one hydrogel.

45. The system of claim 44, wherein a hydrogel comprises one or more of agarose, hyaluronic acid, chitosan, alginate, collagen, dextran, pectin, carrageenan, polylysine, gelatin, hyaluronic acid, fibrin, and methylcellulose.

46. The system of claim 45, wherein the hydrogel comprises hyaluronic acid.

47. The system of claim 46, wherein the hyaluronic acid is or comprises methacrylated HA (MeHA).

48. The system of claim 45, wherein the hydrogel is or comprises agarose.

49. The system of claim 48, wherein the concentration of agarose is at about 0.25-30%, about 0.25%-3%, about 0.5%-3%, about 1-20%, about 1.5-10%, about 2-9%, about 2.5-8%, or about 3-7%.

50. The system of claim 49, wherein the concentration of agarose is at about 3%.

51. The system of claim 42, wherein an inner surface of the biocompatible construct comprises one or more extracellular matrix (ECM) components.

52. The system of claim 51, wherein the ECM component comprises collagen, laminin, fibronectin, hyaluronic acid, or a combination thereof.

53. The system of claim 51, wherein the ECM comprises collagen.

54. The system of claim 53, wherein the collagen is at a concentration of about 0.1-10 mg/ml or 0.1-9 mg/ml.

55. The system of claim 53, wherein the collagen is at a concentration of about 1 mg/ml.

56. The system of claim 27, wherein the plurality of astrocytes is seeded at least once in the system.

57. The system of claim 56, wherein the plurality of astrocytes is seeded twice in the system.

58. The system of claim 56, wherein the plurality of astrocytes is seeded at a cell density of about 0.1 million cells/ml to about 10 million cells/ml.

59. The system of claim 56, wherein the plurality of astrocytes is seeded at a cell density of about 1 million cells/ml.

60. The system of claim 27, wherein the plurality of astrocytes comprises at least 500 cells, at least 1000 cells, at least 5000 cells, at least 10,000 cells, at least 15,000 cells, at least 20,000 cells, at least 40,000 cells, at least 80,000 cells, at least 100,000 cells, or at least 500,000 cells.

61. The system of claim 27, wherein the system further comprises one or more additional cells or components.

62. The system of claim 61, wherein an additional cell comprises an endothelial cell.

63. The system of claim 61, wherein the one or more additional cells or components induces vascularization.

64. The system of claim 61, wherein the additional cell or component is introduced into the system:
(i) concurrently with, before or after seeding of the plurality of astrocytes;
(ii) during formation of an astrocyte bundle by the plurality of astrocytes; and/or
(iii) after formation of an astrocyte bundle by the plurality of astrocytes.

65. A method of manufacturing a system comprising
(a) a biocompatible construct comprising a matrix and having a first end, a second end and a body; and
(b) associating the biocompatible construct with a plurality of astrocytes derived from at least one gingiva-derived mesenchymal stem cell (GDMSC).

66. The method of claim 65, wherein the method comprises maintaining the system under conditions that promotes growth of at least one astrocyte in the plurality of astrocytes.

67. The method of claim 65, wherein the method comprises maintaining the system under conditions that maintain viability of at least one astrocyte in the plurality of astrocytes.

68. The method of claim 65, wherein the method comprises forming an aggregate of at least a portion of the plurality of astrocytes.

69. (canceled)

70. (canceled)

71. A method of promoting cell migration, comprising,
providing a system comprising: (a) a plurality of astrocytes derived from at least one gingiva-derived mesenchymal stem cell; and (b) a biocompatible construct comprising a first end, a second end and a body comprising a luminal core,
wherein the system is implanted in a subject, and the method is characterized in that when the system is implanted in a subject, the system promotes migration of one or more cells through or along the luminal core of the biocompatible construct.

72. The method of claim 71, wherein the subject is a mammal.

73. The method of claim 71, wherein the system is implanted:
(i) at, near or within a brain lesion in a subject;
(ii) at, near or within an area in the brain with insufficient neurons or neuronal connections, or with damaged neurons or neuronal connections;
(iii) at, near or within a subventricular zone, an endogenous rostral migratory stream or a neurogenic niche; and/or
(iv) at, near or within a region of a brain affected by a brain injury, a neurodegenerative disease or disorder, or a neurodevelopmental disease or disorder.

74. The method of claim 71, wherein the method promotes the migration of one or more endogenous host cells.

75. The method of claim 74, wherein the endogenous host cells comprise a neural precursor cell, a neuroblast, a neuron, a progenitor cell, a glial cell, an astrocyte, and/or an endothelial cell.

76. The method of claim 71, wherein the method promotes the migration of one or more cells provided in the system, or one or more cells derived from, differentiated from or a progenitor of a cell provided in the system.

77. The method of claim 71, wherein migration of one or more cells occurs within the system, throughout the system, out of the system or into the system.

78. The method of claim 71, wherein migration of one or more cells occurs toward the site of implantation, or away from the site of implantation.

79. (canceled)

80. A method of treating a neurodegenerative disorder or a neurological disorder in a subject, comprising implanting into the subject a system comprising: (a) a plurality of astrocytes derived from at least one gingiva-derived mesenchymal stem cell (GDMSC); and (b) a biocompatible construct comprising a matrix and having a first end, a second end and a body.

81. The method of claim 80, wherein a neurodegenerative disorder comprises a disorder with injury or degeneration to one or more neurons.

82. The method of claim 80, wherein a neurodegenerative disorder comprises brain injury or spinal cord injury.

83. The method of claim 82, wherein brain injury comprises acute brain injury, degenerative brain injury, traumatic brain injury (TBI), or chronic brain injury.

84. The method of claim 83, wherein a neurological disorder comprises: Parkinson's, Alzheimer's, Huntington's, prion disease, motor neuron disease, spinocerebellar ataxia, spinal muscular atrophy, amyotrophic lateral sclerosis (ALS), encephalitis, epilepsy, head and brain malformations, or hydrocephalus.

85. The method of claim 80, wherein the subject is a mammal.

86. The method of claim 80, wherein the subject is a human.

87. The method of claim 85, wherein the system is implanted into the brain of the subject.

88. The method of claim 86, wherein the system is implanted:
(i) at, near or within a brain lesion in a subject;
(ii) at, near or within an area in the brain with insufficient neurons or neuronal connections, or with damaged neurons or neuronal connections;
(iii) at, near or within a subventricular zone, an endogenous rostral migratory stream or a neurogenic niche; and/or
(iv) at, near or within a region of a brain affected by a brain injury, a neurodegenerative disease or disorder, or a neurodevelopmental disease or disorder.

89. The method of claim 80, wherein the system is characterized in that when implanted into an organism it ameliorates or reduces severity of one or more symptoms of a disorder.

90. The method of claim 80, wherein the system is characterized in that when implanted into an organism it promotes migration of one or more cells.

91. The method of claim 90, wherein migration of one or more cells is compared to a comparator.

92. The method of claim 91, wherein a comparator comprises an organism implanted with an otherwise similar system without a plurality of astrocytes; or without a plurality of astrocytes derived from at least one GDMSC.

93. The method of claim 90, wherein the method promotes the migration of comprises one or more endogenous host cells.

94. The method of claim 93, wherein an endogenous host cell comprises a neural precursor cell, a neuroblast, a neuron, a progenitor cell, a glial cell, an astrocyte, and/or an endothelial cell.

95. The method of claim 90, wherein the method promotes the migration of: one or more cells provided in the system; or one or more cells derived from, differentiated from or a progenitor of a cell provided in the system.

96. The method of claim 90, wherein migration of one or more cells occurs within the system, throughout the system, out of the system or into the system.

97. The method of claim 90, wherein migration of one or more cells occurs to the site of implantation, or away from the site of implantation.

98. (canceled)

99. A kit comprising, a system which comprises:
(a) a plurality of astrocytes derived from at least one gingiva-derived mesenchymal stem cell (GDMSC); and
(b) a biocompatible construct comprising a matrix and having a first end, a second end and a body; and
(c) instructions for using the same.

100. (canceled)
